SEQUENCE LISTING (70 sequences)

SEQ ID NO: 1 - protein, 9 aa, synthetic construct
  SITE 1: note = N-terminus is modified
  SITE 1: note = D-Phenylalanine
  SITE 6: note = Gly or meGly
  SITE 8..9: note = joined by a reduced peptide bond
  SITE 9: note = thiazoline-4-carboxylic acid
  SITE 9: note = C-terminally amidated
FQWAVXHLX

SEQ ID NO: 2 - protein, 9 aa, synthetic construct
  SITE 1: note = D-Phenylalanine
  SITE 9: note = thiazoline-4-carboxylic acid
  SITE 9: note = C-terminally amidated
FQWAVGHLX

SEQ ID NO: 3 - protein, 9 aa, synthetic construct
  SITE 1: note = D-3-(2-naphtyl)alanine
  SITE 9: note = thiazoline-4-carboxylic acid
  SITE 9: note = C-terminally amidated
XQWAVGHLX

SEQ ID NO: 4 - protein, 9 aa, synthetic construct
  SITE 1: note = D-Phenylalanine
  SITE 9: note = thiazoline-4-carboxylic acid
  SITE 9: note = C-terminally amidated
FQWAVGHLX

SEQ ID NO: 5 - protein, 9 aa, synthetic construct
  SITE 1: note = D-Phenylalanine
  SITE 5: note = tranexamic acid, tert-leucine
  SITE 9: note = thiazoline-4-carboxylic acid
  SITE 9: note = C-terminally amidated
FQWAXGHLX

SEQ ID NO: 6 - protein, 9 aa, synthetic construct
  SITE 5: note = tranexamic acid, tert-leucine
  MOD_RES 7: note = N-Methylhistidine
  SITE 9: note = thiazoline-4-carboxylic acid
  SITE 9: note = C-terminally amidated
FQWAXGHLX

SEQ ID NO: 7 - protein, 9 aa, synthetic construct
  SITE 1: note = D-Phenylalanine
  SITE 8..9: note = joined by a reduced peptide bond
  SITE 9: note = thiazoline-4-carboxylic acid
  SITE 9: note = C-terminally amidated
FHWAVGHLX

SEQ ID NO: 8 - protein, 9 aa, synthetic construct
  SITE 1: note = D-Phenylalanine
  SITE 5: note = tranexamic acid, tert-leucine
  MOD_RES 7: note = N-Methylhistidine
  SITE 9: note = thiazoline-4-carboxylic acid
  SITE 9: note = C-terminally amidated
FHWAXGHLX

SEQ ID NO: 9 - protein, 9 aa, synthetic construct
  SITE 1: note = D-Phenylalanine
  SITE 5: note = tranexamic acid, tert-leucine
  SITE 8..9: note = joined by a reduced peptide bond
  SITE 9: note = thiazoline-4-carboxylic acid
  SITE 9: note = C-terminally amidated
FQWAXGHLX

SEQ ID NO: 10 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 10: note = thiazoline-4-carboxylic acid
  SITE 10: note = C-terminally amidated
XFQWAVGHLX

SEQ ID NO: 11 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-3-(2-naphtyl)alanine
  SITE 10: note = thiazoline-4-carboxylic acid
  SITE 10: note = C-terminally amidated
XXQWAVGHLX

SEQ ID NO: 12 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  MOD_RES 8: note = N-Methylhistidine
  SITE 10: note = thiazoline-4-carboxylic acid
  SITE 10: note = C-terminally amidated
XFQWAVGHLX

SEQ ID NO: 13 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 6: note = tranexamic acid, tert-leucine
  SITE 10: note = thiazoline-4-carboxylic acid
  SITE 10: note = C-terminally amidated
XFQWAXGHLX

SEQ ID NO: 14 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 6: note = tranexamic acid, tert-leucine
  MOD_RES 8: note = N-Methylhistidine
  SITE 10: note = thiazoline-4-carboxylic acid
  SITE 10: note = C-terminally amidated
XFQWAXGHLX

SEQ ID NO: 15 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 6: note = tranexamic acid, tert-leucine
  MOD_RES 8: note = N-Methylhistidine
  SITE 10: note = thiazoline-4-carboxylic acid
  SITE 10: note = C-terminally amidated
XFHWAXGHLX

SEQ ID NO: 16 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 9..10: note = joined by a reduced peptide bond
  SITE 10: note = thiazoline-4-carboxylic acid
  SITE 10: note = C-terminally amidated
XFHWAVGHLX

SEQ ID NO: 17 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 6: note = tranexamic acid, tert-leucine
  SITE 9..10: note = joined by a reduced peptide bond
  SITE 10: note = thiazoline-4-carboxylic acid
  SITE 10: note = C-terminally amidated
XFQWAXGHLX

SEQ ID NO: 18 - protein, 9 aa, synthetic construct
  SITE 1: note = D-Phenylalanine
  SITE 5: note = tranexamic acid, tert-leucine
  SITE 9: note = thiazoline-4-carboxylic acid
  SITE 9: note = C-terminally amidated
FQWAXGHLX

SEQ ID NO: 19 - protein, 9 aa, synthetic construct
  SITE 1: note = D-Phenylalanine
  SITE 9: note = thiazoline-4-carboxylic acid
  SITE 9: note = C-terminally amidated
FQWAVGHLX

SEQ ID NO: 20 - protein, 9 aa, synthetic construct
  SITE 1: note = D-Phenylalanine
  MOD_RES 7: note = N-Methylhistidine
  SITE 9: note = thiazoline-4-carboxylic acid
  SITE 9: note = C-terminally amidated
FQWAVGHLX

SEQ ID NO: 21 - protein, 9 aa, synthetic construct
  SITE 1: note = D-Phenylalanine
  SITE 3: note = citrulline
  SITE 9: note = thiazoline-4-carboxylic acid
  SITE 9: note = C-terminally amidated
FQXAVGHLX

SEQ ID NO: 22 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 4: note = aplha-methyltryptophan
  SITE 6: note = tranexamic acid, tert-leucine
  SITE 9..10: note = joined by a reduced peptide bond
  SITE 10: note = thiazoline-4-carboxylic acid
  SITE 10: note = C-terminally amidated
XFQXAXGHLX

SEQ ID NO: 23 - protein, 11 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 6: note = tranexamic acid, tert-leucine
  MOD_RES 7: note = meGly
  SITE 10..11: note = joined by a reduced peptide bond
  SITE 11: note = thiazoline-4-carboxylic acid
  SITE 11: note = C-terminally amidated
XFQWAXNGHLX

SEQ ID NO: 24 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 4: note = 7-Fluoro-tryptophan
  SITE 10: note = thiazoline-4-carboxylic acid
  SITE 10: note = C-terminally amidated
XFQXAVGHLX

SEQ ID NO: 25 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 6: note = tranexamic acid, tert-leucine
  MOD_RES 8: note = N-Methylhistidine
  SITE 10: note = thiazoline-4-carboxylic acid
  SITE 10: note = C-terminally amidated
XFHWAXGHLX

SEQ ID NO: 26 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 4: note = 5-methyltryptophan
  SITE 10: note = thiazoline-4-carboxylic acid
  SITE 10: note = C-terminally amidated
XFQXAVGHLX

SEQ ID NO: 27 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 4: note = 2-methyltryptophan
  SITE 10: note = thiazoline-4-carboxylic acid
  SITE 10: note = C-terminally amidated
XFQXAVGHLX

SEQ ID NO: 28 - protein, 11 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  MOD_RES 7: note = meGly
  SITE 10..11: note = joined by a reduced peptide bond
  SITE 11: note = C-terminally amidated
XFQWAVNGHLP

SEQ ID NO: 29 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 10: note = thiazoline-4-carboxylic acid
  SITE 10: note = C-terminally amidated
XFQWAVGHFX

SEQ ID NO: 30 - protein, 9 aa, synthetic construct
  SITE 1: note = D-Phenylalanine
  SITE 5: note = 2,3-dehydro-Valine
  SITE 9: note = thiazoline-4-carboxylic acid
  SITE 9: note = C-terminally amidated
FQWAXGHLX

SEQ ID NO: 31 - protein, 9 aa, synthetic construct
  SITE 1: note = D-Phenylalanine
  SITE 5: note = L-cyclopropylglycine
  SITE 9: note = thiazoline-4-carboxylic acid
  SITE 9: note = C-terminally amidated
FQWAXGHLX

SEQ ID NO: 32 - protein, 9 aa, synthetic construct
  SITE 1: note = D-Phenylalanine
  SITE 5: note = cyclobutaneacetic acid
  SITE 9: note = thiazoline-4-carboxylic acid
  SITE 9: note = C-terminally amidated
FQWAXGHLX

SEQ ID NO: 33 - protein, 9 aa, synthetic construct
  SITE 1: note = D-Phenylalanine
  SITE 3: note = 5-Fluoro-tryptophan
  SITE 9: note = thiazoline-4-carboxylic acid
  SITE 9: note = C-terminally amidated
FQXAVGHLX

SEQ ID NO: 34 - protein, 9 aa, synthetic construct
  SITE 1: note = D-Phenylalanine
  SITE 3: note = 6-methyltryptophan
  SITE 9: note = thiazoline-4-carboxylic acid
  SITE 9: note = C-terminally amidated
FQXAVGHLX

SEQ ID NO: 35 - protein, 9 aa, synthetic construct
  SITE 1: note = D-Phenylalanine
  SITE 3: note = 5-hydroxytryptophan
  SITE 9: note = thiazoline-4-carboxylic acid
  SITE 9: note = C-terminally amidated
FQXAVGHLX

SEQ ID NO: 36 - protein, 9 aa, synthetic construct
  SITE 1: note = D-Phenylalanine
  SITE 3: note = 6-Fluoro-tryptophan
  SITE 9: note = thiazoline-4-carboxylic acid
  SITE 9: note = C-terminally amidated
FQXAVGHLX

SEQ ID NO: 37 - protein, 9 aa, synthetic construct
  SITE 1: note = D-Phenylalanine
  SITE 3: note = 7-Fluoro-tryptophan
  SITE 9: note = thiazoline-4-carboxylic acid
  SITE 9: note = C-terminally amidated
FQXAVGHLX

SEQ ID NO: 38 - protein, 9 aa, synthetic construct
  SITE 1: note = D-Phenylalanine
  SITE 3: note = 4-Fluoro-tryptophan
  SITE 9: note = thiazoline-4-carboxylic acid
  SITE 9: note = C-terminally amidated
FQXAVGHLX

SEQ ID NO: 39 - protein, 9 aa, synthetic construct
  SITE 1: note = D-Phenylalanine
  SITE 3: note = 5-methyltryptophan
  SITE 9: note = thiazoline-4-carboxylic acid
  SITE 9: note = C-terminally amidated
FQXAVGHLX

SEQ ID NO: 40 - protein, 9 aa, synthetic construct
  SITE 1: note = D-Phenylalanine
  SITE 3: note = 4-methyltryptophan
  SITE 9: note = thiazoline-4-carboxylic acid
  SITE 9: note = C-terminally amidated
FQXAVGHLX

SEQ ID NO: 41 - protein, 9 aa, synthetic construct
  SITE 1: note = D-Phenylalanine
  SITE 3: note = D-2,3,4,9-tetrahydro-1H-pyrido[3,4-b]indol-3-carbox ylic acid
  SITE 9: note = thiazoline-4-carboxylic acid
  SITE 9: note = C-terminally amidated
FQXAVGHLX

SEQ ID NO: 42 - protein, 9 aa, synthetic construct
  SITE 1: note = D-Phenylalanine
  SITE 3: note = 7-methyltryptophan
  SITE 9: note = thiazoline-4-carboxylic acid
  SITE 9: note = C-terminally amidated
FQXAVGHLX

SEQ ID NO: 43 - protein, 9 aa, synthetic construct
  SITE 1: note = D-Phenylalanine
  SITE 3: note = 2-methyltryptophan
  SITE 9: note = thiazoline-4-carboxylic acid
  SITE 9: note = C-terminally amidated
FQXAVGHLX

SEQ ID NO: 44 - protein, 9 aa, synthetic construct
  SITE 1: note = D-Phenylalanine
  SITE 3: note = 7-Azatryptophan
  SITE 9: note = thiazoline-4-carboxylic acid
  SITE 9: note = C-terminally amidated
FQXAVGHLX

SEQ ID NO: 45 - protein, 9 aa, synthetic construct
  SITE 1: note = D-Phenylalanine
  SITE 3: note = 2-aminooctanoic acid
  SITE 9: note = thiazoline-4-carboxylic acid
  SITE 9: note = C-terminally amidated
FQXAVGHLX

SEQ ID NO: 46 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 6: note = tranexamic acid, tert-leucine
  SITE 10: note = thiazoline-4-carboxylic acid
  SITE 10: note = C-terminally amidated
XFQWAXGHLX

SEQ ID NO: 47 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 10: note = thiazoline-4-carboxylic acid
  SITE 10: note = C-terminally amidated
XFQWAVGHLX

SEQ ID NO: 48 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  MOD_RES 8: note = N-Methylhistidine
  SITE 10: note = thiazoline-4-carboxylic acid
  SITE 10: note = C-terminally amidated
XFQWAVGHLX

SEQ ID NO: 49 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 4: note = citrulline
  SITE 10: note = thiazoline-4-carboxylic acid
  SITE 10: note = C-terminally amidated
XFQXAVGHLX

SEQ ID NO: 50 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 6: note = 2,3-dehydro-Valine
  SITE 10: note = thiazoline-4-carboxylic acid
  SITE 10: note = C-terminally amidated
XFQWAXGHLX

SEQ ID NO: 51 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 6: note = L-cyclopropylglycine
  SITE 10: note = thiazoline-4-carboxylic acid
  SITE 10: note = C-terminally amidated
XFQWAXGHLX

SEQ ID NO: 52 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 6: note = cyclobutaneacetic acid
  SITE 10: note = thiazoline-4-carboxylic acid
  SITE 10: note = C-terminally amidated
XFQWAXGHLX

SEQ ID NO: 53 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 4: note = 5-Fluoro-tryptophan
  SITE 10: note = thiazoline-4-carboxylic acid
  SITE 10: note = C-terminally amidated
XFQXAVGHLX

SEQ ID NO: 54 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 4: note = 6-methyltryptophan
  SITE 10: note = thiazoline-4-carboxylic acid
  SITE 10: note = C-terminally amidated
XFQXAVGHLX

SEQ ID NO: 55 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 4: note = 5-hydroxytryptophan
  SITE 10: note = thiazoline-4-carboxylic acid
  SITE 10: note = C-terminally amidated
XFQXAVGHLX

SEQ ID NO: 56 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 4: note = 6-Fluoro-tryptophan
  SITE 10: note = thiazoline-4-carboxylic acid
  SITE 10: note = C-terminally amidated
XFQXAVGHLX

SEQ ID NO: 57 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 4: note = 7-Fluoro-tryptophan
  SITE 10: note = thiazoline-4-carboxylic acid
  SITE 10: note = C-terminally amidated
XFQXAVGHLX

SEQ ID NO: 58 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 4: note = 4-Fluoro-tryptophan
  SITE 10: note = thiazoline-4-carboxylic acid
  SITE 10: note = C-terminally amidated
XFQXAVGHLX

SEQ ID NO: 59 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 4: note = 5-methyltryptophan
  SITE 10: note = thiazoline-4-carboxylic acid
  SITE 10: note = C-terminally amidated
XFQXAVGHLX

SEQ ID NO: 60 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 4: note = 4-methyltryptophan
  SITE 10: note = thiazoline-4-carboxylic acid
  SITE 10: note = C-terminally amidated
XFQXAVGHLX

SEQ ID NO: 61 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 4: note = D-2,3,4,9-tetrahydro-1H-pyrido[3,4-b]indol-3-carbox ylic acid
  SITE 10: note = thiazoline-4-carboxylic acid
  SITE 10: note = C-terminally amidated
XFQXAVGHLX

SEQ ID NO: 62 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 4: note = 7-methyltryptophan
  SITE 10: note = thiazoline-4-carboxylic acid
  SITE 10: note = C-terminally amidated
XFQXAVGHLX

SEQ ID NO: 63 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 4: note = 2-methyltryptophan
  SITE 10: note = thiazoline-4-carboxylic acid
  SITE 10: note = C-terminally amidated
XFQXAVGHLX

SEQ ID NO: 64 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 4: note = 7-Azatryptophan
  SITE 10: note = thiazoline-4-carboxylic acid
  SITE 10: note = C-terminally amidated
XFQXAVGHLX

SEQ ID NO: 65 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 4: note = 2-aminooctanoic acid
  SITE 10: note = thiazoline-4-carboxylic acid
  SITE 10: note = C-terminally amidated
XFQXAVGHLX

SEQ ID NO: 66 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  SITE 9..10: note = joined by a reduced peptide bond
  SITE 10: note = thiazoline-4-carboxylic acid
  SITE 10: note = C-terminally amidated
XFQWAVGHLX

SEQ ID NO: 67 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-3-(2-naphtyl)alanine
  SITE 9..10: note = joined by a reduced peptide bond
  SITE 10: note = thiazoline-4-carboxylic acid
  SITE 10: note = C-terminally amidated
XXQWAVGHLX

SEQ ID NO: 68 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-2,3,4,9-tetrahydro-1H-pyrido[3,4-b]indol-3-carbox ylic acid
  SITE 9..10: note = joined by a reduced peptide bond
  SITE 10: note = thiazoline-4-carboxylic acid
  SITE 10: note = C-terminally amidated
XXQWAVGHLX

SEQ ID NO: 69 - protein, 10 aa, synthetic construct
  SITE 1: note = N-terminus is modified with 1,4,7,10-tetraazacyclododecane- 1,4,7,10-tetraacetic acid
  SITE 1: note = 4-amino-1-carboxymethyl-piperidine
  SITE 2: note = D-Phenylalanine
  MOD_RES 7: note = meGly
  SITE 9..10: note = joined by a reduced peptide bond
  SITE 10: note = thiazoline-4-carboxylic acid
  SITE 10: note = C-terminally amidated
XFQWAVGHLX

SEQ ID NO: 70 - protein, 11 aa, synthetic construct
  SITE 1: note = N-terminus is modified with 1,4,7,10-tetraazacyclododecane- 1,4,7,10-tetraacetic acid
  MOD_RES 1: note = Cysteic acid
  SITE 2: note = 4-amino-1-carboxymethyl-piperidine
  SITE 3: note = D-3-(2-naphtyl)alanine
  SITE 10..11: note = joined by a reduced peptide bond
  SITE 11: note = thiazoline-4-carboxylic acid
  SITE 11: note = C-terminally amidated
CXXQWAVGHLX

(19) United States
(12) Patent Application Publication
(10) Pub. No.: US 2025/0195702 A1
(43) Pub. Date: Jun. 19, 2025

(54) RADIOLABELED COMPOUNDS FOR IN VIVO IMAGING OF GASTRIN-RELEASING PEPTIDE RECEPTOR (GRPR) AND TREATMENT OF GRPR-RELATED DISORDERS

(71) Applicants: PROVINCIAL HEALTH SERVICES AUTHORITY, Vancouver (CA); THE UNIVERSITY OF BRITISH COLUMBIA, Vancouver (CA)

(72) Inventors: Kuo-Shyan LIN, Richmond (CA); François BÉNARD, Vancouver (CA); Lei WANG, Vancouver (CA); Zhengxing ZHANG, Langley (CA); Ivica BRATANOVIC, Port Moody (CA); Chengcheng ZHANG, Richmond (CA)

(21) Appl. No.: 19/043,780

(22) Filed: Feb. 3, 2025

Related U.S. Application Data
(63) Continuation of application No. 18/512,708, filed on Nov. 17, 2023, now Pat. No. 12,246,076.
Application No. 18/512,708 is a continuation of application No. PCT/CA2023/050401, filed on Mar. 24, 2023.
(60) Provisional application No. 63/323,831, filed on Mar. 25, 2022.

(51) Int. Cl. A61K 51/08

(52) U.S. Cl. CPC A61K 51/088; A61K 2121/00; A61K 2123/00

(57) ABSTRACT

There is provided peptidic compounds of Formula I, A or B(R^{rad}ₙ₆-[linker]-R^{L}-Xaa¹-Xaa²-Xaa³-Xaa⁴-Xaa⁵-Xaa⁶-Xaa⁷-Xaa⁸-ψ-Xaa⁹-NH₂). Xaa¹ is D-Phe, Cpa, D-Cpa, Nal, D-Nal, 2-Nal, or D-2-Nal; Xaa² is Asn, Gln, Hse, Cit or His. Xaa³ is Trp, Bta, Trp(Me), Trp(7-Me), Trp(6-Me), Trp(5-Me), Trp(4-Me), Trp(2-Me), Trp(7-F), Trp(6-F), Trp(5-F), Trp(4-F), Trp(5-OH), or αMe-Trp. Xaa⁴ is Ala or Ser. Xaa⁵ is Val, Cpg, or Tle. Xaa⁶ is Gly, NMe-Gly, or D-Ala. Xaa⁷ is His or NMe-His. Xaa⁸ is Leu or Phe. Xaa⁹-NH₂ is a C-terminally amidated amino acid residue selected from Pro, 4-oxa-L-Pro, Me₂Thz, or Thz. ψ represents a peptide bond or reduced peptide bond joining Xaa⁸ to Xaa⁹. R^{rad}ₙ₆ is 1-5 radiolabeling groups. There is also provided the use of such compounds as imaging agents or therapeutic agents.

### CROSS-REFERENCE TO RELATED APPLICATIONS

This application is a continuation of U.S. patent application Ser. No. 18/512,708, filed Nov. 17, 2023, which is a continuation of International Application No. PCT/CA2023/050401, filed Mar. 24, 2023, which claims priority to U.S. Provisional Patent Application No. 63/323,831, filed on Mar. 25, 2022, the contents of each of which is hereby incorporated by reference in its entirety for all purposes.

### REFERENCE TO SEQUENCE LISTING SUBMITTED ELECTRONICALLY

The contents of the electronic sequence listing (A9TH_020_02US_SeqList_ST26.xml; Size: 203,261 bytes; and Date of Creation: Jan. 16, 2025) are herein incorporated by reference in its entirety.

### FIELD OF INVENTION

The present invention relates to radiolabelled compounds for in vivo imaging or treatment of diseases or conditions characterized by expression of the gastrin-releasing peptide receptor.

### BACKGROUND OF THE INVENTION

Gastrin-releasing peptide receptor (GRPR) is a G protein-coupled receptor of the bombesin (BBN) receptor family (Roesler & Schwartsmann. 2012. Front Endocrinol (Lausanne) 3:159; Bitar & Zhu. 1993. Gastroenterology. 105:1672-1680; Weber. 2009. Curr Opin Endocrinol Diabetes Obes. 16:66-71). Together with its endogenous ligand, gastrin-releasing peptide (GRP), GRPR is involved in synaptic plasticity, emotional and feeding behavior, hormone secretion, smooth muscle contraction, and cell proliferation (ibid.). In normal conditions, the expression of GRPR is restricted to the central nervous system, pancreas, adrenal cortex and gastrointestinal tract (Jensen, et al. 2008. Pharmacol Rev. 60:1-42). GRPR is also implicated in neoplastic progression, with overexpression of GRPR having been reported in many cancer subtypes including lung, head and neck, colon, kidney, ovarian, breast and prostate cancers (Cornelio, et al. 2007. Ann Oncol. 18:1457-1466). This ectopic expression in cancers makes it an attractive target for personalized therapies.

BBN is a 14 amino acid GRPR binding peptide (Lin, et al. 2004. Bioconjugate Chemistry. Vol 15. American Chemical Society pages 1416-1423; Inkster, et al. 2013 Bioorganic Med Chem Lett. 23:3920-3926; Mansi, et al. 2016 J Nucl Med. 57:67S-72S; Bodei, et al. 2007. ¹⁷⁷Lu-AMBA Bombesin analogue in hormone refractory prostate cancer patients: a phase I escalation study with single-cycle administrations. In: JOINT EANM-EORTC Symposium; Sah, et al. 2015 J Nucl Med. 56:372-378; Zang, et al. 2018 Clin Nucl Med. 43:663-669; Nock, et al. 2017 J Nucl Med. 58:75-80; Maina, et al. 2016 Eur J Nucl Med Mol Imaging 43:964-973). BBN derivatives have been radiolabeled for imaging with single photon emission computed tomography (SPECT), positron emission tomography (PET), and have also been radiolabeled for therapy with beta and alpha emitters (Maina, et al. PET Clin. 2017; 12:297-309; Lin, et al. 2004. Bioconjugate Chemistry. Vol 15. American Chemical Society pages 1416-1423; Inkster, et al. 2013 Bioorganic Med Chem Lett. 23:3920-3926). Often, a radiolabelled group is appended directly onto the structure or via a linker at the N-terminus, while modifications at the C-terminus dictate agonist/antagonist properties. For targeting GRPR, antagonists are preferred since agonists have been shown to induce gastrointestinal adverse events (Bodei, et al. 2007. ¹⁷⁷Lu-AMBA Bombesin analogue in hormone refractory prostate cancer patients: a phase I escalation study with single-cycle administrations. In: JOINT EANM-EORTC Symposium). Examples of GRPR antagonists evaluated in the clinic include: ⁶⁸Ga-RM2, ⁶⁸Ga-SB3, ⁶⁸Ga-NeoBOMB1, ⁶⁸Ga-RM26, ¹⁸F-BAY-864367, and ⁶⁴Cu-CB-TE2A-AR06 (Mansi, et al. 2016 J Nucl Med. 57:67S-72S; Sah, et al. 2015 J Nucl Med. 56:372-378; Zang, et al. 2018 Clin Nucl Med. 43:663-669; Nock, et al. 2017 J Nucl Med. 58:75-80; Maina, et al. 2016 Eur J Nucl Med Mol Imaging 43:964-973; Kahkonen, et al. Clin Cancer Res. 2013; 19:5434-5443, Kahkonen, et al. Clin Cancer Res. 2013; 19:5434-5443; Baum, et al. 2007 Journal of Nuclear Medicine 48, 79P-79P).

High pancreas uptake is the major limitation of currently reported GRPR-targeting radioligands. In a study, the high pancreas uptake of ⁶⁸Ga-labeled AMBA was up to 54.9 SUV (SUV: standard uptake value) (Baum, et al. 2007 Journal of Nuclear Medicine 48, 79P-79P). In addition, ⁶⁸Ga-labeled RM2 was also reported to show high uptake in pancreas (Kurth, et al. 2020. European journal of nuclear medicine and molecular imaging 47, 123-135; Minamimoto, et al. 2016 J Nucl Med. 57:557-562). It has also been reported that radiolabeled NeoBOMB1 showed high pancreas uptake in both PC-3 tumor-bearing mice and prostate cancer patients (Nock, et al. 2017 J Nucl Med. 58:75-80).

Another limitation for most of the reported GRPR-targeting ligands is their in vivo metabolic instability (Bakker, et al. 2018 Molecular imaging and biology 20, 973-983; Rousseau, et al. 2020 Journal of Labelled Compounds and Radiopharmaceuticals 63, 56-64) due to enzymatic degradation by neutral endopeptidase (NEP) (Nock, et al. 2014 J Nucl Med. 55:121-127). His¹²-Leu¹³, Trp⁸-Ala⁹ and Gln⁷-Trp⁸ were reported to be the main cleavage sites within the AMBA's sequence, and Trp⁸-Ala⁹, Ala⁹-Val¹⁰ and Gln⁷-Trp⁸ were considered to be the cleavage sites of RM2 (Kahkonen, et al. 2013 Clin Cancer Res. 19:5434-5443; Linder et al. 2009 Bioconjugate chemistry 20, 1171-1178).

There remains an unmet need in the field for improved tracers for the non-invasive in-vivo imaging of the GRPR. Such tracers are useful for the diagnosis of disorders related to aberrant/ectopic expression of GRPR, including but not limited to cancer (e.g. prostate cancer). There also remains an unmet need for improved radiotherapeutic agents for treatment of diseases/disorders related to aberrant/ectopic expression of GRPR, including but not limited to cancer (e.g. prostate cancer). In particular, there is a need for GRPR-targeting radioligands (for imaging and/or therapy) with lower pancreas uptake, and useful stability in vivo.

No admission is necessarily intended, nor should it be construed, that any of the preceding information constitutes prior art against the present invention.

### SUMMARY

In one aspect, this disclosure provides a peptidic compound of Formula I (defined below). Such compounds may have lower pancreas uptake than prior art bombesin analogs as well as useful stability in vivo for imaging and/or radiotherapy.

### BRIEF DESCRIPTION OF THE DRAWINGS

The features of the invention will become apparent from the following description in which reference is made to the appended drawings wherein:

FIG. 1 shows representative maximum-intensity-projection PET images of ⁶⁸Ga-LW01025, ⁶⁸Ga-LW01029, ⁶⁸Ga-LW01107, ⁶⁸Ga-LW01108, ⁶⁸Ga-LW01110, ⁶⁸Ga-LW01142, ⁶⁸Ga-LW01158 and ⁶⁸Ga-LW01102 in mice bearing PC-3 tumor xenografts. The images were acquired at 1 h post-injection.

FIG. 2 shows representative radio-HPLC chromatograms of ⁶⁸Ga-LW01025 extracted from mouse urine and plasma samples.

FIG. 3 shows representative radio-HPLC chromatograms from analysis of intact fraction of ⁶⁸Ga-LW01029 in mouse plasma (A) and urine (B) samples collected at 15 min post-injection.

FIG. 4 shows representative radio-HPLC chromatograms of ⁶⁸Ga-LW01107 extracted from mouse urine and plasma samples.

FIG. 5 shows representative radio-HPLC chromatograms of ⁶⁸Ga-LW01108 extracted from mouse urine and plasma samples.

FIG. 6 shows representative radio-HPLC chromatograms of ⁶⁸Ga-LW01110 extracted from mouse urine and plasma samples.

FIG. 7 shows representative radio-HPLC chromatograms of ⁶⁸Ga-LW01142 extracted from mouse urine and plasma samples.

FIG. 8 shows representative radio-HPLC chromatograms of ⁶⁸Ga-LW01102 extracted from mouse urine and plasma samples.

FIGS. 9A-9C show representative data of ⁶⁸Ga-LW01045, including a representative maximum-intensity-projection PET image of ⁶⁸Ga-LW01045 in a mouse bearing a PC-3 tumor xenograft (FIG. 9A); a representative displacement curve of [¹²⁵I-Tyr⁴]Bombesin by Ga-LW01045 generated using GRPR-expressing PC-3 cells (FIG. 9B); and radio-HPLC chromatograms of ⁶⁸Ga-LW01045 extracted from mouse urine and plasma samples (FIG. 9C).

FIGS. 10A-10B show representative data of ⁶⁸Ga-LW01059, including a maximum-intensity-projection PET image of ⁶⁸Ga-LW01059 in a mouse bearing a PC-3 tumor xenograft (FIG. 10A); and a displacement curve of [¹²⁵I-Tyr⁴]Bombesin by Ga-LW01059 generated using GRPR-expressing PC-3 cells (FIG. 10B).

FIGS. 11A-11C show representative data of ⁶⁸Ga-LW01090, including a maximum-intensity-projection PET image of ⁶⁸Ga-LW01090 in a mouse bearing a PC-3 tumor xenograft (FIG. 11A); a displacement curve of [¹²⁵I-Tyr⁴]Bombesin by Ga-LW01090 generated using GRPR-expressing PC-3 cells (FIG. 11B); and radio-HPLC chromatograms of ⁶⁸Ga-LW01090 extracted from mouse urine and plasma samples (FIG. 11C).

FIGS. 12A-12B show representative data of ⁶⁸Ga-LW01117, including a maximum-intensity-projection PET image of ⁶⁸Ga-LW01117 in a mouse bearing a PC-3 tumor xenograft (FIG. 12A); and a representative displacement curve of [¹²⁵I-Tyr⁴]Bombesin by Ga-LW01117 generated using GRPR-expressing PC-3 cells (FIG. 12B).

FIG. 13 shows a representative maximum-intensity-projection PET image of ⁶⁸Ga-LW02045 in a mouse bearing a PC-3 tumor xenograft.

### DETAILED DESCRIPTION

As used herein, the terms “comprising,” “having”, “including” and “containing,” and grammatical variations thereof, are inclusive or open-ended and do not exclude additional, unrecited elements and/or method steps, even if a feature/component defined as a part thereof consists or consists essentially of specified feature(s)/component(s). The term “consisting essentially of” if used herein in connection with a compound, composition, use or method, denotes that additional elements and/or method steps may be present, but that these additions do not materially affect the manner in which the recited compound, composition, method or use functions. The term “consisting of” if used herein in connection with a feature of a compound, composition, use or method, excludes the presence of additional elements and/or method steps in that feature. A compound, composition, use or method described herein as comprising certain elements and/or steps may also, in certain embodiments consist essentially of those elements and/or steps, and in other embodiments consist of those elements and/or steps, whether or not these embodiments are specifically referred to. A use or method described herein as comprising certain elements and/or steps may also, in certain embodiments consist essentially of those elements and/or steps, and in other embodiments consist of those elements and/or steps, whether or not these embodiments are specifically referred to.

A reference to an element by the indefinite article “a” does not exclude the possibility that more than one of the elements is present, unless the context clearly requires that there be one and only one of the elements. The singular forms “a”, “an”, and “the” include plural referents unless the context clearly dictates otherwise. The use of the word “a” or “an” when used herein in conjunction with the term “comprising” may mean “one,” but it is also consistent with the meaning of “one or more,” “at least one” and “one or more than one.”

In this disclosure, the recitation of numerical ranges by endpoints includes all numbers subsumed within that range including all whole numbers, all integers and, where suitable, all fractional intermediates (e.g., 1 to 5 may include 1, 1.5, 2, 2.75, 3, 3.80, 4, and 5 etc.).

Unless otherwise specified, “certain embodiments”, “various embodiments”, “an embodiment” and similar terms includes the particular feature(s) described for that embodiment either alone or in combination with any other embodiment or embodiments described herein, whether or not the other embodiments are directly or indirectly referenced and regardless of whether the feature or embodiment is described in the context of a method, product, use, composition, compound, et cetera.

As used herein, the terms “treat”, “treatment”, “therapeutic” and the like includes ameliorating symptoms, reducing disease progression, improving prognosis and reducing recurrence.

As used herein, the term “diagnostic agent” includes an “imaging agent”. As such, a “diagnostic radionuclide” includes radionuclides that are suitable for use in imaging agents.

The term “subject” refers to an animal (e.g. a mammal or a non-mammal animal). The subject may be a human or a non-human primate. The subject may be a laboratory mammal (e.g., mouse, rat, rabbit, hamster and the like). The subject may be an agricultural animal (e.g., equine, ovine, bovine, porcine, camelid and the like) or a domestic animal (e.g., canine, feline and the like). In some embodiments, the subject is a human.

The compounds disclosed herein may also include base-free forms, solvates, salts or pharmaceutically acceptable salts thereof. Unless otherwise specified or indicated, the compounds claimed and described herein are meant to include all racemic mixtures and all individual enantiomers or combinations thereof, whether or not they are explicitly represented herein.

The compounds disclosed herein may be shown as having one or more charged groups, may be shown with ionizable groups in an uncharged (e.g. protonated) state or may be shown without specifying formal charges. As will be appreciated by the person of skill in the art, the ionization state of certain groups within a compound (e.g. without limitation, COOH, and the like) is dependent, inter alia, on the pKa of that group and the pH at that location. For example, but without limitation, a carboxylic acid group (i.e. COOH) would be understood to usually be deprotonated (and negatively charged) at neutral pH and at most physiological pH values, unless the protonated state is stabilized.

As used herein, the terms “salt” and “solvate” have their usual meaning in chemistry. As such, when the compound is a salt or solvate, it is associated with a suitable counter-ion. It is well known in the art how to prepare salts or to exchange counter-ions. Generally, such salts can be prepared by reacting free acid forms of these compounds with a stoichiometric amount of a suitable base (e.g. without limitation, Na, Ca, Mg, or K hydroxide, carbonate, bicarbonate, or the like), or by reacting free base forms of these compounds with a stoichiometric amount of a suitable acid. Such reactions are generally carried out in water or in an organic solvent, or in a mixture of the two. Counter-ions may be changed, for example, by ion-exchange techniques such as ion-exchange chromatography. All zwitterions, salts, solvates and counter-ions are intended, unless a particular form is specifically indicated.

In certain embodiments, the salt or counter-ion may be pharmaceutically acceptable, for administration to a subject. As used herein, “pharmaceutically acceptable” means suitable for in vivo use in a subject, and is not necessarily restricted to therapeutic use, but also includes diagnostic use. More generally, with respect to any pharmaceutical composition disclosed herein, non-limiting examples of suitable excipients include any suitable buffers, stabilizing agents, salts, antioxidants, complexing agents, tonicity agents, cryoprotectants, lyoprotectants, suspending agents, emulsifying agents, antimicrobial agents, preservatives, chelating agents, binding agents, surfactants, wetting agents, non-aqueous vehicles such as fixed oils, or polymers for sustained or controlled release. See, for example, Berge et al. 1977. (J. Pharm Sci. 66:1-19), or Remington—The Science and Practice of Pharmacy, 21st edition (Gennaro et al editors. Lippincott Williams & Wilkins Philadelphia), each of which is incorporated by reference in its entirety.

As used herein, the expression “Cₙ” where n is an integer (e.g. 1, 2, 3, 4, 5, 6, 7, 8, 9, 10, 11, 12, 13, 14, 15, 16, 17, 18, 19, 20, and the like) or where n is defined as a range of integers (e.g. 1-20, 1-18, 2-15, 3-20, and the like) refers to the number of carbons in a compound, R-group, L-group, or substituent, or refers to the number of carbons plus heteroatoms in a compound, R-group, L-group, or substituent. A range of integers includes all integers in the range; e.g. the range 1-20 includes the integers 1, 2, 3, 4, 5, 6, 7, 8, 9, 10, 11, 12, 13, 14, 15, 16, 17, 18, 19, and 20. Unless otherwise defined, heteroatoms may include any, some or all possible heteroatoms. For example, in some embodiments, the heteroatoms may be selected from N, O, S, P and Se. In some embodiments, the heteroatoms are selected from N, S, or O. Such embodiments are non-limiting. The alternative expression “Cy-Cz”, where y and z are integers (e.g. C₃-C₁₅ and the like), is equivalent to “Cₙ” where n is a range of integers from y to z.

The terms “alkyl”, “alkylenyl”, “alkenylenyl”, and “alkynylenyl” have their usual meanings in organic chemistry. For example, an “alkyenylenyl” has at least one carbon-carbon double bond, and may have any number of carbon-carbon single bonds. Similarly, an “alkynylenyl” has at least one carbon-carbon triple bond, and may have any number of carbon-carbon single bonds. The expressions “alkylenyl, alkenylenyl and/or alkynylenyl” and “alkylenyl, alkenylenyl or alkynylenyl” are intended to be equivalent and each includes hydrocarbon chains that can have any reasonable number or combination of carbon-carbon single bonds, double bonds, and triple bonds. These hydrocarbon chains can be linear, branched, cyclic, or any combination of linear and branched, linear and cyclic, cyclic and branched, branched and cyclic, or linear, branched and cyclic. Cyclic hydrocarbons may be nonaromatic, partially aromatic, or aromatic. Unless otherwise specified, the term “cyclic” includes single rings, multiple non-fused rings, fused rings, bridged rings, and combinations thereof.

The expression “wherein any carbon . . . is optionally independently replaced by N, S, or O” and other similar expressions means that the defined hydrocarbon (e.g. “alkyl”, “alkylenyl”, “alkenylenyl”, or “alkynylenyl”) includes zero, one, more than one, or any reasonable combination of two or more heteroatoms selected from N, S, and O. The above expression therefore expands the defined hydrocarbon to additionally encompass heteroalkyls, heteroalkylenyls, heteroalkenylenyls, and heteroalkynylenyls, etc. The person of skill in the art would understand that various combinations of different heteroatoms may be used. The expression “wherein any carbon bonded to two other carbons is optionally independently replaced by N, S, or O” and other similar expressions means that any carbon in the defined hydrocarbon bonded to two other carbons (e.g. the underlined carbon in —C—C—C—), whether those bonds are single, double, or triple bonds, may be a heteroatom, but excludes heteroatoms bonded to other heteroatoms (e.g. excludes —C—N—S—, —S—S—N—, —N—S—C—, and the like).

Various R-groups (e.g. R¹, R², R³, etc.) and L-groups (e.g. L¹, L², L³, etc.) are defined in this disclosure. L-groups generally refer to linkages (e.g. —S—, —NH—C(O)—, —C(O)—NH—, —N(alkyl)-C(O)—, —C(O)—N(alkyl)-, —NH—C(O)—NH—, —NH—C(S)—NH—,

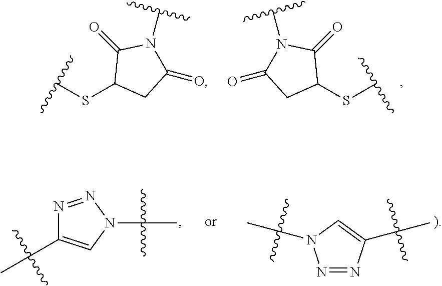

If unspecified, the size of an R-group or L-group is what would be considered reasonable to the person of skill in the art. For example, but without limitation, if unspecified, the size of an alkyl may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 89, 90, 91, 92, 93, 94, 95, 96, 97, 98, 99, 100 or more than 100 carbons in length, subject to the common general knowledge of the person of skill in the art. Further, but without limitation, if unspecified, the size of a heteroalkyl may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 89, 90, 91, 92, 93, 94, 95, 96, 97, 98, 99, 100 or more than 100 carbons and heteroatoms in length, subject to the common general knowledge of the person of skill in the art. In the context of the expression “alkyl, alkenyl or alkynyl” and similar expressions, the “alkyl” would be understood to be a saturated alkyl, and the “alkenyl” and the “alkynyl” would be understood to be unsaturated.

As used herein, in the context of an alkyl/heteroalkyl group of a compound, the term “linear” may be used as it is normally understood to a person of skill in the art and generally refers to a chemical entity that comprises a skeleton or main chain that does not split off into more than one contiguous chain. Non-limiting examples of linear alkyls include methyl, ethyl, n-propyl, and n-butyl.

As used herein, the term “branched” may be used as it is normally understood to a person of skill in the art and generally refers to a chemical entity that comprises a skeleton or main chain that splits off into more than one contiguous chain. The portions of the skeleton or main chain that split off in more than one direction may be linear, cyclic or any combination thereof. Non-limiting examples of a branched alkyl group include tert-butyl and isopropyl.

The term “alkylenyl” refers to a divalent analog of an alkyl group. In the context of the expression “alkylenyl, alkenylenyl and/or alkynylenyl”, and similar expressions, the “alkylenyl” would be understood to be a saturated alkylenyl, and the “alkenylenyl” and the “alkynylenyl” would be understood to be unsaturated. The term “heteroalkylenyl” refers to a divalent analog of a heteroalkyl group. The term “heteroalkenylenyl” refers to a divalent analog of a heteroalkenyl group. The term “heteroalkynylenyl” refers to a divalent analog of a heteroalkynyl group.

As used herein, the term “saturated” when referring to a chemical entity may be used as it is normally understood to a person of skill in the art and generally refers to a chemical entity that comprises only single bonds, and may include linear, branched, and/or cyclic groups. Non-limiting examples of a saturated C₁-C₂₀ alkyl group may include methyl, ethyl, n-propyl, i-propyl, sec-propyl, n-butyl, i-butyl, sec-butyl, t-butyl, n-pentyl, i-pentyl, sec-pentyl, t-pentyl, n-hexyl, i-hexyl, 1,2-dimethylpropyl, 2-ethylpropyl, 1-methyl-2-ethylpropyl, I-ethyl-2-methylpropyl, 1,1,2-trimethylpropyl, 1,1,2-triethylpropyl, 1,1-dimethylbutyl, 2,2-dimethylbutyl, 2-ethylbutyl, 1,3-dimethylbutyl, 2-methylpentyl, 3-methylpentyl, sec-hexyl, t-hexyl, n-heptyl, i-heptyl, sec-heptyl, t-heptyl, n-octyl, i-octyl, sec-octyl, t-octyl, n-nonyl, i-nonyl, sec-nonyl, t-nonyl, n-decyl, i-decyl, sec-decyl, t-decyl, cyclopropanyl, cyclobutanyl, cyclopentanyl, cyclohexanyl, cycloheptanyl, cyclooctanyl, cyclononanyl, cyclodecanyl, and the like. Unless otherwise specified, a C₁-C₂₀ alkylenyl therefore encompasses, without limitation, all divalent analogs of the above-listed saturated alkyl groups.

As used herein, the term “unsaturated” when referring to a chemical entity may be used as it is normally understood to a person of skill in the art and generally refers to a chemical entity that comprises at least one double or triple bond, and may include linear, branched, and/or cyclic groups. Non-limiting examples of a C₂-C₂₀ alkenyl group may include vinyl, allyl, isopropenyl, I-propene-2-yl, 1-butene-1-yl, I-butene-2-yl, I-butene-3-yl, 2-butene-1-yl, 2-butene-2-yl, octenyl, decenyl, cyclopropenyl, cyclobutenyl, cyclopentenyl, cyclohexenyl, cycloheptenyl, cyclooctenyl, cyclononanenyl, cyclodecanenyl, and the like. Unless otherwise specified, a C₁-C₂₀ alkenylenyl therefore encompasses, without limitation, all divalent analogs of the above-listed alkenyl groups. Non-limiting examples of a C₂-C₂₀ alkynyl group may include ethynyl, propynyl, butynyl, pentynyl, hexynyl, heptynyl, octynyl, nonynyl, decynyl, and the like. Unless otherwise specified, a C₁-C₂₀ alkynylenyl therefore encompasses, without limitation, all divalent analogs of the above-listed alkynyl groups.

Non-limiting examples of non-aromatic cyclic groups include cylcopropyl, cyclobutyl, cyclopentyl, cyclohexyl, and the like. Non-limiting examples of non-aromatic heterocyclic groups include aziridinyl, azetidinyl, diazetidinyl, pyrrolidinyl, pyrrolinyl, piperidinyl, piperazinyl, imidazolinyl, pyrazolidinyl, imidazolydinyl, phthalimidyl, succinimidyl, oxiranyl, tetrahydropyranyl, oxetanyl, dioxanyl, thietanyl, thiepinyl, morpholinyl, oxathiolanyl, and the like.

Unless further specified, an “aryl” group includes both single aromatic rings as well as fused rings containing at least one aromatic ring. non-limiting examples of C₃-C₂₀ aryl groups include phenyl (Ph), pentalenyl, indenyl, naphthyl and azulenyl. Non-limiting examples of aromatic heterocyclic groups of similar size include pyrrolyl, imidazolyl, pyrazolyl, pyridinyl, pyridazinyl, pyrimidinyl, pirazinyl, quinolinyl, isoquinolinyl, acridinyl, indolyl, isoindolyl, indolizinyl, purinyl, carbazolyl, indazolyl, phthalazinyl, naphthyridinyl, quinoxalinyl, quinazolinyl, cinnolinyl, pteridinyl, phenanthridinyl, phenazinyl, phenanthrolinyl, perimidinyl, furyl, dibenzofuryl, xanthenyl, benzofuryl, thiophenyl, thianthrenyl, benzothiophenyl, phosphorinyl, phosphinolinyl, phosphindolyl, thiazolyl, oxazolyl, isoxazolyl, and the like.

As used herein, the term “substituted” is used as it would normally be understood to a person of skill in the art and generally refers to a compound or chemical entity that has one chemical group replaced with a different chemical group. Unless otherwise specified, a substituted alkyl, alkylenyl, alkenylenyl, or alkynylenyl has one or more hydrogen atom(s) independently replaced with an atom that is not hydrogen. For example, chloromethyl is a non-limiting example of a substituted alkyl, more particularly an example of a substituted methyl. Aminoethyl is another non-limiting example of a substituted alkyl, more particularly an example of a substituted ethyl. Unless otherwise specified, a substituted compound or group (e.g. R-group or L-group) may be substituted with any chemical group reasonable to the person of skill in the art. For example, but without limitation, a hydrogen bonded to a carbon or heteroatom (e.g. N) may be substituted with halide (e.g. F, I, Br, Cl), amine, amide, oxo, hydroxyl, thiol, phosphate, phosphonate, sulfate, SO₂H, SO₃H, alkyls, heteroalkyls, aryl, heteroaryl, ketones, carboxaldehyde, carboxylates, carboxamides, nitriles, monohalomethyl, dihalomethyl or trihalomethyl. In some embodiments, each carbon may be independently substituted or unsubstituted with oxo, hydroxyl, sulfhydryl, amine, amide, urea, halogen, guanidino, carboxylic acid, sulfonic acid, sulfinic acid, or phosphoric acid. In some embodiments, the amide substituent is —C(O)—NH₂.

As used herein, the term “unsubstituted” is used as it would normally be understood to a person of skill in the art. Non-limiting examples of unsubstituted alkyls include methyl, ethyl, tert-butyl, pentyl and the like. The expression “optionally substituted” is used interchangeably with the expression “unsubstituted or substituted”. The expression “optionally independently substituted” means that each location may be substituted or may not be substituted, and when substituted each substituent may be the same or different.

In the structures provided herein, hydrogen may or may not be shown. In some embodiments, hydrogens (whether shown or implicit) may be protium (i.e. ¹H), deuterium (i.e. ²H) or combinations of ¹H and ²H. Methods for exchanging ¹H with ²H are well known in the art. For solvent-exchangeable hydrogens, the exchange of ¹H with ²H occurs readily in the presence of a suitable deuterium source, without any catalyst. The use of acid, base or metal catalysts, coupled with conditions of increased temperature and pressure, can facilitate the exchange of non-exchangeable hydrogen atoms, generally resulting in the exchange of all ¹H to ²H in a molecule.

The term “Xaa” refers to an amino acid residue in a peptide chain or an amino acid that is otherwise part of a compound. Amino acids have both an amino group and a carboxylic acid group, either or both of which can be used for covalent attachment. In attaching to the remainder of the compound, the amino group and/or the carboxylic acid group may be converted to an amide or other structure; e.g. a carboxylic acid group of a first amino acid is converted to an amide (i.e. a peptide bond) when bonded to the amino group of a second amino acid. As such, Xaa may have the formula —N(R^{a})R^{b}C(O)—, where R^{a} and R^{b} are R-groups. R^{a} will typically be hydrogen or alkyl (e.g. methyl) or R^{a} and R^{b} may form a cyclic structure. The amino acid residues of a peptide may comprise typical peptide (amide) bonds and may further comprise bonds between side chain functional groups and the side chain or main chain functional group of another amino acid. For example, the side chain carboxylate of one amino acid residue in the peptide (e.g. Asp, Glu, etc.) may be bonded to and the amine of another amino acid residue in the peptide (e.g. Dap, Dab, Orn, Lys). Further details are provided below. Unless otherwise indicated, “Xaa” may be any amino acid, including a proteinogenic or nonproteinogenic amino acid. Non-limiting examples of nonproteinogenic amino acids are shown in Table 1 and include: D-amino acids (including without limitation any D-form of the following amino acids), ornithine (Orn), 3-(1-naphtyl)alanine (Nal), 3-(2-naphtyl)alanine (2-Nal), α-aminobutyric acid, norvaline, norleucine (Nle), homonorleucine, beta-(1,2,3-triazol-4-yl)-L-alanine, 1,2,4-triazole-3-alanine, Phe(4-F), Phe(4-Cl), Phe(4-Br), Phe(4-I), Phe(4-NH₂), Phe(4-NO₂), homoarginine (hArg), 2-amino-4-guanidinobutyric acid (Agb), 2-amino-3-guanidinopropionic acid (Agp), B-alanine, 4-aminobutyric acid, 5-aminovaleric acid, 6-aminohexanoic acid, 7-aminoheptanoic acid, 8-aminooctanoic acid, 9-aminononanoic acid, 10-aminodecanoic acid, 2-aminooctanoic acid, 2-amino-3-(anthracen-2-yl)propanoic acid, 2-amino-3-(anthracen-9-yl) propanoic acid, 2-amino-3-(pyren-1-yl)propanoic acid, Trp(5-Br), Trp(5-OCH₃), Trp(6-F), Trp(5-OH) or Trp(CHO), 2-aminoadipic acid (2-Aad), 3-aminoadipic acid (3-Aad), propargylglycine (Pra), homopropargylglycine (Hpg), beta-homopropargylglycine (Bpg), 2,3-diaminopropionic acid (Dap), 2,4-diaminobutyric acid (Dab), azidolysine (Lys(N₃)), azido-ornithine (Orn(N₃)), 2-amino-4-azidobutanoic acid Dab(N₃), Dap(N₃), 2-(5′-azidopentyl)alanine, 2-(6′-azidohexyl)alanine, 4-amino-1-carboxymethyl-piperidine (Pip), 4-(2-aminoethyl)-1-carboxymethyl-piperazine (Acp), tranexamic acid, tert-leucine (Tle), 4-chlorophenylalanine (Cpa), thiazoline-4-carboxylic acid (Thz), αMe-Trp, p-aminomethylaniline-diglycolic acid (pABzA-DIG), 4-amino-1-carboxymethyl-piperidine (Pip), NH₂(CH₂)₂O(CH₂)₂C(O)OH, NH₂(CH₂)₂[O(CH₂)₂]₂C(O)OH (dPEG2), NH₂(CH₂)₂[O(CH₂)₂]₃C(O)OH, NH₂(CH₂)₂[O(CH₂)₂]₄C(O)OH, NH₂(CH₂)₂[O(CH₂)₂]₅C(O)OH, NH₂(CH₂)₂[O(CH₂)₂]₆C(O)OH, oxazolidine-4-carboxylic acid (4-oxa-L-Pro), β-(3-benzothienyl)alanine (Bta), citrulline (Cit), Trp(Me), Trp (7-Me), Trp(6-Me), Trp(5-Me), Trp(4-Me), Trp(2-Me), Trp(7-F), Trp(5-F), Trp(4-F) or cyclopentylglycine (Cpa). If not specified as an L- or D-amino acid, an amino acid shall be understood to be an L-amino acid.

TABLE 1
List of non-limiting examples of non-proteinogenic amino acids.

| | |
|---|---|
| p-aminomethylaniline-diglycolic acid (pABzA-DIG) | 2-amino-3-(anthracen-2-yl)propanoic |
| | acid |
| ornithine (Orn) | 2-amino-3-(anthracen-9-yl)propanoic |
| | acid |
| 3-(1-naphtyl)alanine (Nal) | 2-amino-3-(pyren-1-yl)propanoic acid |
| 3-(2-naphtyl)alanine (2-Nal) | Trp(5-Br), |
| α-aminobutyric acid | Trp(5-OCH₃), |
| norvaline | Trp(6-F) |
| norleucine (Nle) | Trp(5-OH) |
| homonorleucine | Trp(CHO) |
| beta-(1,2,3-triazol-4-yl)-L-alanine | N^{ε},N^{ε},N^{ε}-trimethyl-lysine |
| 1,2,4-triazole-3-alanine | cysteic acid |
| Phe(4-F), Phe(2-F), Phe(3-F), | 2-aminoadipic acid (2-Aad) |
| Phe(4-Cl), Phe(2-Cl), Phe(3-Cl), | 3-aminoadipic acid (3-Aad) |
| Phe(4-Br), Phe(2-Br), Phe(3-Br), | propargylglycine (Pra) |
| Phe(4-I), Phe(2-I), Phe(2-I), | homopropargylglycine (Hpg) |
| Phe(4-NH₂), Phe(2-NH₂), Phe(3-NH₂), | beta-homopropargylglycine (Bpg) |
| Phe(4-NO₂), Phe(2-NO₂), Phe(2-NO₂), | 2,3-diaminopropionic acid (Dap) |
| homoarginine (hArg) | 2,4-diaminobutyric acid (Dab) |
| 4-(2-aminoethyl)-1-carboxymethyl-piperazine (Acp) | azidolysine (Lys(N₃)) |
| 2-(5′-azidopentyl)alanine, 2-(6′-azidohexyl)alanine | azido-ornithine (Orn(N₃)) |
| 2-amino-4-guanidinobutyric acid (Agb) | amino-4-azidobutanoic acid Dab(N₃) |
| 2-amino-3-guanidinopropionic acid (Agp) | tranexamic acid |
| β-alanine | 4-amino-1-carboxymethyl-piperidine |
| | (Pip) |
| 4-aminobutyric acid | NH₂(CH₂)₂O(CH₂)₂C(O)OH |
| 5-aminovaleric acid | NH₂(CH₂)₂[O(CH₂)₂]₂C(O)OH (dPEG2) |
| 6-aminohexanoic acid | NH₂(CH₂)₂[O(CH₂)₂]₃C(O)OH |
| 7-aminoheptanoic acid | NH₂(CH₂)₂[O(CH₂)₂]₄C(O)OH |
| 8-aminooctanoic acid | NH₂(CH₂)₂[O(CH₂)₂]₅C(O)OH |
| 9-aminononanoic acid | NH₂(CH₂)₂[O(CH₂)₂]₆C(O)OH |
| 10-aminodecanoic acid | citrulline (Cit) |
| 2-aminooctanoic acid | β-(3-benzothienyl)alanine (Bta) |
| tert-leucine (Tle) | oxazolidine-4-carboxylic acid |
| | (4-oxa-L-Pro) |
| 4-chlorophenylalanine (Cpa) | cyclopentylglycine (Cpg) |
| thiazoline-4-carboxylic acid (Thz) | any N-methylated version of a |
| | proteinogenic amino acid or |
| | non-proteinogenic amino acid in this |
| | Table |
| αMe-Trp | any D-amino acid of a proteinogenic |
| | amino acid or any D-amino acid of a |
| | non-proteinogrenic amino acid in this |
| | Table |
| Trp(Me) | Trp(7-Me) |
| Trp(6-Me) | Trp(5-Me) |
| Trp(4-Me) | Trp(2-Me) |
| Trp(7-F) | Trp(5-F) |
| Tpi(2,3,4,9-tetrahydro-1H-pyrido[3,4-b]indol-3-carboxylic | cyclobutylglycine |
| acid) | |
| Trp(4-F) | 5,5-dimethyl-1,3-thiazolidine-4-carboxylic |
| | acid (Me₂Thz) |
| 7-Aza (7-azatryptophan) | 2,3-dehyrdo-Val |
| | cyclopropylglycine |

The wavy line “ [symbol] ” symbol shown through or at the end of a bond in a chemical formula (e.g. in the definitions L¹ or R^{alb} of Formula I) is intended to define the group on one side of the wavy line, without modifying the definition of the structure on the opposite side of the wavy line. Where an R-group or L-group is bonded on two or more sides, any atoms shown outside the wavy lines are intended to clarify orientation of the defined group. As such, only the atoms between the two wavy lines constitute the definition of the R-group or L-group. When atoms are not shown outside the wavy lines (e.g. L¹), or for a chemical group shown without wavy lines but does have bonds on multiple sides (e.g. —C(O)NH—, and the like), the chemical group should be read from left to right matching the orientation in the formula that the group relates to; e.g. for formula —R^{a}—R^{b}—R^{c}—, the definition of R^{b} as —C(O)NH— would be incorporated into the formula as —R^{a}—C(O)NH—R^{c}— not as —R^{a}—NHC(O)—R^{c}—.

In various aspects, there is disclosed a peptidic compound, wherein the compound has the structure of Formula I or is a salt or solvate of Formula I defined as follows:

R^{rad}ₙ₆-[linker]-R^{L}-Xaa¹-Xaa²-Xaa³-Xaa⁴-Xaa⁵-Xaa⁶-Xaa⁷-Xaa⁸-ψ-Xaa⁹-NH₂ (I)

wherein:
Xaa¹ is an N-terminal amino acid residue selected from D-Phe, Cpa (4-chlorophenylalanine), D-Cpa, Nal (3-(1-naphthyl)alanine), D-Nal, 2-Nal (3-(2-naphthyl)alanine), or D-2-Nal;
Xaa² is Asn, Gln, homoserine (Hse), citrulline (Cit) or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(5-OH) or aMe-Trp;
Xaa⁴ is Ala or Ser;
Xaa⁵ is Val, Cpg (cyclopentylglycine) or tert-leucine (Tle);
Xaa⁶ is Gly, NMe-Gly, or D-Ala;
Xaa⁷ is His or NMe-His;
Xaa⁸ is Leu, D-Pro, or Phe;
Xaa⁹-NH₂ is a C-terminally amidated amino acid residue selected from Pro, Phe, 4-oxa-L-Pro (oxazolidine-4-carboxylic acid), Me₂Thz (5,5-dimethyl-1,3-thiazolidine-4-carboxylic acid), or Thz (thiazoline-4-carboxylic acid);
ψ is a peptide bond or reduced peptide bond between Xaa⁸ and Xaa⁹;
excluding compounds in which Xaa², Xaa³, Xaa⁵, and Xaa⁷ are Gln, Trp, Val, and His, respectively,
in which Ly is a reduced peptide bond;
R^{L} is —C(O)—, —NH—C(O)—, or —NH—C(S)—;
the linker is a linear or branched chain of n1 units of -L¹R¹— and/or -(L¹)₂R¹—, wherein:
   n1 is 1-20;
   each R¹ is, independently, a linear, branched, and/or cyclic Cₙ₂ alkylenyl, alkenylenyl and/or alkynylenyl, wherein each n2 is independently 1-20, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid;
   L¹ bonds to carbon, wherein each L¹ is independently —S—, —N(R²)C(O)—, —C(O)N(R²)—, —NH—C(O)—NH—, —NH—C(S)—NH—,

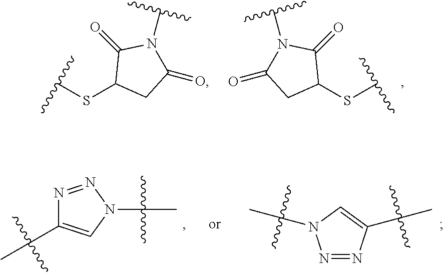

and
R² is H, methyl or ethyl; and
an albumin binder (R^{alb}) is optionally bonded to an L¹ of the linker, wherein the albumin binder is:
   —(CH₂)ₙ₃—CH₃ wherein n3 is 8-20;
   —(CH₂)ₙ₄—C(O)OH wherein n4 is 8-20;

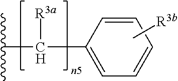

wherein n5 is 1-4 and R^{3a} is H or methyl, and R^{3b} is I, Br, F, Cl, H, OH, OCH₃, NH₂, NO₂ or C₁-C₆ alkyl; or

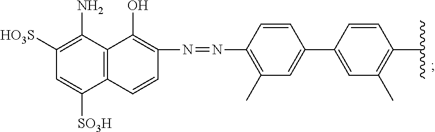 [SMILES: *c1ccc(-c2ccc(N=Nc3ccc4c(S(=O)(=O)O)cc(S(=O)(=O)O)c(N)c4c3O)c(C)c2)cc1C]

n6 is 1-5; and
each R^{rad} is a radiolabeling group bonded to or incorporating an L¹ of the linker, wherein each radiolabeling group is independently: a radiometal chelator; an aryl or heteroaryl substituted with a radiohalogen; a prosthetic group containing a trifluoroborate; a prosthetic group containing a silicon-fluorine-acceptor moiety; or a prosthetic group containing a fluorophosphate, fluorosulfate, sulfonyl fluoride, or a combination thereof.

In another embodiment, the peptidic compound may be a compound with the structure of Formula A or is a salt or solvate of Formula A as follows:

R^{rad}ₙ₆-[linker]-R^{L}-Xaa¹-Xaa²-Xaa³-Xaa⁴-Xaa⁵-Xaa⁶-Xaa⁷-Xaa⁸-ψ-Xaa⁹-NH₂ (Formula A),

wherein:
Xaa¹ is an N-terminal amino acid residue selected from D-Phe, 4-chlorophenylalanine (Cpa), D-Cpa, 3-(1-naphthyl)alanine (Nal), D-Tpi, D-Nal, 3-(2-naphthyl)alanine (2-Nal), or D-2-Nal;
Xaa² is Asn, Gln, homoserine (Hse), citrulline (Cit) or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(5-OH), Tpi, 7-Aza, or aMe-Trp;
Xaa⁴ is Ala or Ser;
Xaa⁵ is Val, 2,3-dehydro-Val, Cpg (cyclopentylglycine), cyclopropylglycine, cyclobutylglycine, or tert-leucine (Tle);
Xaa⁶ is Gly, NMe-Gly, or D-Ala;
Xaa⁷ is His or NMe-His;
Xaa⁸ is Leu, D-Pro, or Phe;
Xaa⁹-NH₂ is a C-terminally amidated amino acid residue selected from Pro, Phe, oxazolidine-4-carboxylic acid (4-oxa-L-Pro), Me₂Thz (5,5-dimethyl-1,3-thiazolidine-4-carboxylic acid), or thiazoline-4-carboxylic acid (Thz);
ψ represents a peptide bond or reduced peptide bond joining Xaa⁸ to Xaa⁹; excluding compounds in which Xaa², Xaa³, Xaa⁵, and Xaa⁷ are Gln, Trp, Val, and His, respectively, in which L is a reduced peptide bond; R^{L} is —C(O)—, —NH—C(O)—, or —NH—C(S)—;
the linker is a linear or branched chain of n1 units of -L¹R¹— and/or -(L¹)₂R¹—, wherein:
   n1 is 1-20; each R¹ is, independently, a linear, branched, and/or cyclic Cₙ₂ alkylenyl, alkenylenyl and/or alkynylenyl, wherein each n2 is independently 1-20, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid;
   L¹ bonds to carbon, wherein each L¹ is independently —S—, —N(R²)C(O)—, —C(O)N(R²)—, —NH—C(O)—NH—, —NH—C(S)—NH—,

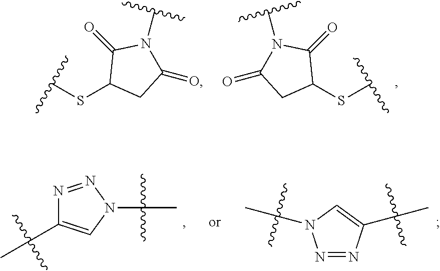

and R² is H, methyl or ethyl; and an albumin binder (R^{alb}) is optionally bonded to an L¹ of the linker, wherein the albumin binder is: —(CH₂)ₙ₃—CH₃ wherein n3 is 8-20; —(CH₂)ₙ₄—C(O)OH wherein n4 is 8-20;

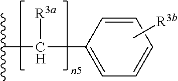

wherein n5 is 1-4 and R^{3a} is H or methyl, and R^{3b} is I, Br, F, Cl, H, OH, OCH₃, NH₂, NO₂ or C₁-C₆ alkyl; or

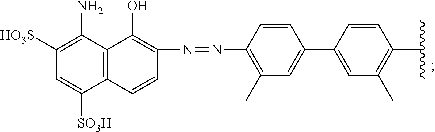 [SMILES: *c1ccc(-c2ccc(N=Nc3ccc4c(S(=O)(=O)O)cc(S(=O)(=O)O)c(N)c4c3O)c(C)c2)cc1C]

n6 is 1-5; and
each R^{rad} is a radiolabeling group bonded to or incorporating an L¹ of the linker, wherein each radiolabeling group is independently: a radiometal chelator; an aryl or heteroaryl substituted with a radiohalogen; a prosthetic group containing a trifluoroborate; a prosthetic group containing a silicon-fluorine-acceptor moiety; or a prosthetic group containing a fluorophosphate, fluorosulfate, sulfonyl fluoride, or a combination thereof.

In another embodiment, the invention may include a peptidic compound where Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), or D-2-Nal; Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(5-OH), or αMe-Trp; and Xaa⁵ is Val, Cpg (cyclopentylglycine), or tert-leucine (Tle).

In another specific embodiment, Xaa¹ is an N-terminal amino acid residue selected from D-Phe, or D-2-Nal. In another specific embodiment, Xaa² is Gln, or His. In another specific embodiment, Xaa⁵ is Val, or tert-leucine (Tle). In another specific embodiment, Xaa⁶ is Gly, or NMe-Gly. In another specific embodiment, Xaa⁹-NH₂ is a C-terminally amidated amino acid residue selected from Pro or thiazoline-4-carboxylic acid (Thz). In another specific embodiment, Xaa¹ is an N-terminal amino acid residue selected from D-Phe, or D-2-Nal; Xaa² is Gln, or His; Xaa⁴ is Ala; Xaa⁵ is Val, or tert-leucine (Tle); Xaa⁶ is Gly, or NMe-Gly; Xaa⁸ is Leu; and Xaa⁹-NH₂ is a C-terminally amidated amino acid residue selected from Pro or thiazoline-4-carboxylic acid (Thz).

In another specific embodiment, Xaa³ is β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(5-OH), Tpi, 7-Aza, or αMe-Trp. In another specific embodiment, L is a peptide bond. In another specific embodiment, Xaa⁹ is Thz. In another specific embodiment, Xaa² is His. In another specific embodiment, wherein Xaa³ is Trp. In another specific embodiment, Xaa⁵ is Tle. In another specific embodiment, Xaa⁷ is NMe-His.

In another specific embodiment of the peptidic compounds described herein, L is a peptide bond; Xaa⁹ is Thz; Xaa² is His; Xaa³ is Trp; Xaa⁵ is Tle; and Xaa⁷ is NMe-His.

In another embodiment, Xaa³ is αMe-Trp. In another embodiment, Xaa⁶ is Gly. In another embodiment, Xaa⁸ is Leu.

In another embodiment, at least one of Xaa¹, Xaa², Xaa³, Xaa⁴, Xaa⁵, Xaa⁶, Xaa⁷, Xaa⁸, or Xaa⁹ is methylated.

In another embodiment, the peptidic compounds of the present invention may have the structure of Formula B or is a salt or solvate of Formula B, wherein Formula B is as follows:

R^{rad}ₙ₆-[inker]-R^{L}-Xaa¹-Xaa²-Xaa³-Xaa⁴-Xaa⁵-Xaa⁶-Xaa⁷-Xaa⁸-ψ-Xaa⁹-NH₂ (Formula B) wherein:
Xaa¹ is an N-terminal amino acid residue selected from D-Phe, 4-chlorophenylalanine (Cpa), D-Cpa, 3-(1-naphthyl)alanine (Nal), D-Tpi, D-Nal, 3-(2-naphthyl)alanine (2-Nal), or D-2-Nal;
Xaa² is Asn, Gln, homoserine (Hse), citrulline (Cit) or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(5-OH), Tpi, 7-Aza, or αMe-Trp;
Xaa⁴ is Ala or Ser;
Xaa⁵ is Val, 2,3-dehydro-Val, Cpg (cyclopentylglycine), cyclopropylglycine, cyclobuylglycine, or tert-leucine (Tle);
Xaa⁶ is Gly, NMe-Gly, or D-Ala;
Xaa⁷ is His or NMe-His;
Xaa⁸ is Leu, D-Pro, or Phe;
Xaa⁹-NH₂ is a C-terminally amidated amino acid residue selected from Pro, oxazolidine-4-carboxylic acid (4-oxa-L-Pro), Me₂Thz (5,5-dimethyl-1,3-thiazolidine-4-carboxylic acid), or thiazoline-4-carboxylic acid (Thz);
ψ represents a peptide bond or reduced peptide bond joining Xaa⁸ to Xaa⁹;
R^{L} is —C(O)—, —NH—C(O)—, or —NH—C(S)—;
the linker is a linear or branched chain of n1 units of -L¹R¹— and/or -(L¹)₂R¹—, wherein:
   n1 is 1-20;
   each R¹ is, independently, a linear, branched, and/or cyclic Cₙ₂ alkylenyl, alkenylenyl and/or alkynylenyl, wherein each n2 is independently 1-20, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid;
   L¹ bonds to carbon, wherein each L¹ is independently —S—, —N(R²)C(O)—, —C(O)N(R²)—, —NH—C(O)—NH—, —NH—C(S)—NH—,

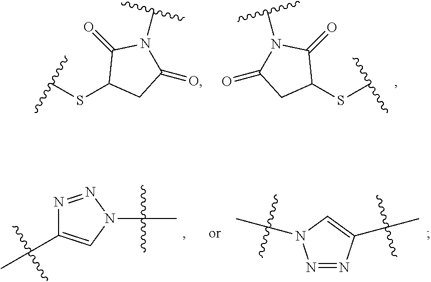

and
R² is H, methyl or ethyl; and
an albumin binder (R^{alb}) is optionally bonded to an L¹ of the linker, wherein the albumin binder is:
—(CH₂)ₙ₃—CH₃ wherein n3 is 8-20;
—(CH₂)ₙ₄—C(O)OH wherein n4 is 8-20;

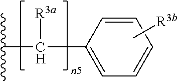

wherein n5 is 1-4 and R^{3a} is H or methyl, and R^{3b} is I, Br, F, Cl, H, OH, OCH₃, NH₂, NO₂ or C₁-C₆ alkyl; or

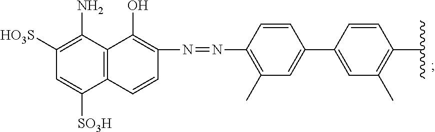 [SMILES: *c1ccc(-c2ccc(N=Nc3ccc4c(S(=O)(=O)O)cc(S(=O)(=O)O)c(N)c4c3O)c(C)c2)cc1C]

n6 is 1-5; and
each R^{rad} is a radiolabeling group bonded to or incorporating an L¹ of the linker, wherein each radiolabeling group is independently: a radiometal chelator; an aryl or heteroaryl substituted with a radiohalogen; a prosthetic group containing a trifluoroborate; a prosthetic group containing a silicon-fluorine-acceptor moiety; or a prosthetic group containing a fluorophosphate, fluorosulfate, sulfonyl fluoride, or a combination thereof.

In a specific embodiment, the compounds of Formulas I, A, or B do not comprise the albumin binder R^{alb} in the linker.

In a specific embodiment, at least one of Xaa¹, Xaa², Xaa³, Xaa⁴, Xaa⁵, Xaa⁶, Xaa⁷, Xaa⁸, or Xaa⁹ is methylated. In another specific embodiment, L is a reduced peptide bond joining Xaa⁸ to Xaa⁹. In another specific embodiment, Xaa¹ is D-Phe; and/or Xaa⁶ is Gly; and/or Xaa⁸ is Leu; and/or Xaa⁹ is Pro, Thz or 4-oxa-L-Pro. In another specific embodiment, Xaa⁶ is Gly or N-methyl-Gly. In another specific embodiment, Xaa⁹ is Thz. In another specific embodiment, Xaa⁹ is Pro. In another embodiment, Xaa¹ is D-phe, Xaa² is Gln, Xaa³ is Trp, Xaa⁴ is Ala, Xaa⁵ is Val, Xaa⁶ Xaa⁶ is Gly or N-methyl-Gly, Xaa⁷ is His, Xaa⁸ is Leu, Xaa⁹ is Thz, and L is a reduced peptide bond joining Xaa⁸ to Xaa⁹. In another specific embodiment, Xaa⁶ is N-methyl-Gly.

In another specific embodiment, the peptidic compound is any compound from Formula I, A, or B, wherein the compounds described in PCT application publication WO2009/109332, which is incorporated by reference in its entirety herein, are excluded. In a specific embodiment, the peptidic compound is any compound from Formula B, wherein the compounds described in PCT application publication WO2009/109332, which is incorporated by reference in its entirety herein, are excluded. In another specific embodiment, the peptidic compound is any compound from Formula I, A, or B, wherein the compounds described in PCT application publication WO2021/068051, which is incorporated by reference in its entirety herein, are excluded. In a specific embodiment, the peptidic compound is any compound from Formula B, wherein the compounds described in PCT application publication WO2021/068051, which is incorporated by reference in its entirety herein, are excluded.

In a specific embodiment, the peptidic compound is any compound described in PCT application publication WO2021/068051, which is incorporated by reference in its entirety herein.

In a specific embodiment, the peptidic compound is any compound from Formula B, wherein the compounds described in Wang, L et al., Molecules, 2022 27, 3777, which is incorporated by reference in its entirety herein, are excluded.

In a specific embodiment, the peptidic compound is any compound described in Wang, L et al., Molecules, 2022 27, 3777, which is incorporated by reference in its entirety herein.

In another specific embodiment, peptidic compounds of Formula I, A, or B exclude R^{rad}ₙ₆-[linker]-R^{L}—, or R^{rad}ₙ₆, or [linker] or R^{L}—.

In another specific embodiment of the peptidic compounds described herein, the radiometal, the radionuclide-bound metal, or the radionuclide-bound metal-containing prosthetic group is: ⁶⁸Ga, ⁶¹Cu, ⁶⁴Cu, ⁶⁷Ga, ^{99m}Tc, ^{110m}In, ¹¹¹In, ⁴⁴Sc, ⁸⁶Y, ⁸⁹Zr, ⁹⁰Nb, ¹⁵²Tb, ¹⁵⁵Tb, [¹⁸F]AlF, ¹³¹I, ¹²³I, ¹²⁴I, and ²⁰³Pb, ⁷²As.

In another specific embodiment of the peptidic compounds described herein, the radiometal, the radionuclide-bound metal, or the radionuclide-bound metal-containing prosthetic group is: ¹⁶⁵Er, ²¹²Bi, ²¹¹At, ¹⁶⁶Ho, ¹⁴⁹Pm, ¹⁵⁹Gd, ¹⁰⁵Rh, ¹⁰⁹Pd, ¹⁹⁸Au, ¹⁹⁹Au, ¹⁷⁵Yb, ¹⁴²Pr, ¹⁷⁷Lu, ¹¹¹In, ²¹³Bi, ⁴⁷Sc, ⁹⁰Y, ²²⁵Ac, ^{117m}Sn, ¹⁵³Sm, ¹⁴⁹Tb, ¹⁶¹Tb, ²²⁴Ra, ²²³Ra, ²¹²Pb, ²²⁷Th, ²²³Ra, ⁷⁷As, ¹⁸⁶Re, ¹⁸⁸Re, ⁶⁷Cu, or ⁶⁴Cu.

In another specific embodiment, the compounds or peptidic compounds may be included in a pharmaceutical composition. In a specific embodiment, the pharmaceutical composition may include one or more compounds from Formula I, A, or B and a pharmaceutically acceptable carrier. In another specific embodiment, the peptidic compound(s) may be bound to or include a radiometal. In a specific embodiment, the radiometal is ¹⁶⁵Er, ²¹²Bi, ²¹¹At, ¹⁶⁶Ho, ¹⁴⁹Pm, ¹⁵⁹Gd, ¹⁰⁵Rh, ¹⁰⁹Pd, ¹⁹⁸Au, ¹⁹⁹Au, ¹⁷⁵Yb, ¹⁴²Pr, ¹⁷⁷Lu, ¹¹¹In, ²¹³Bi, ⁴⁷Sc, ⁹⁰Y, ²²⁵Ac, ^{177m}Sn, ¹⁵³Sm, ¹⁴⁹Tb, ¹⁶¹Tb, ²²⁴Ra, ²²³Ra, ²¹²Pb, ²²⁷Th, ²²³Ra, ⁷⁷As, ¹⁸⁶Re, ¹⁸⁸Re, ⁶⁷Cu, or ⁶⁴Cu.

In another embodiment, the invention may include using the peptidic compounds described herein for imaging methods. In a specific embodiment, the methods may include imaging Gastrin-releasing peptide receptor (GRPR) in a subject, the method comprising: administering to the subject a peptidic compound of any one of Formulas I, A or B; and imaging tissue of the subject. In a specific embodiment, the methods may include the methods of treating cancer in a subject comprising, administering to the subject in need thereof a peptidic compound of any one of Formulas I, A or B and a pharmaceutically acceptable excipient.

In another specific embodiment, the methods may include treating a GRPR-expressing condition or disease. In a specific embodiment, the GRPR-expressing condition or disease may be a psychiatric disorder, neurological disorder, inflammatory disease, prostate cancer, lung cancer, head and neck cancer, colon cancer, kidney cancer, ovarian cancer, liver cancer, pancreatic cancer, breast cancer, glioma or neuroblastoma. In some embodiments, the cancer is prostate cancer.

As described herein, the peptidic moiety -Xaa¹-Xaa²-Xaa³-Xaa⁴-Xaa⁵-Xaa⁶-Xaa⁷-Xaa⁸-ψ-Xaa⁹-NH₂ is the GRPR-targeting moiety of the compound; i.e. it is capable of specifically binding GRPR and potentially producing antagonist effects.

In some embodiments, Xaa¹ is D-Phe. In other embodiments, Xaa¹ is Cpa. In other embodiments, Xaa¹ is D-Cpa. In other embodiments, Xaa¹ is Nal. In other embodiments, Xaa¹ is D-Nal. In other embodiments, Xaa¹ is 2-Nal. In other embodiments, Xaa¹ is D-2-Nal. D-Phe at position Xaa¹ has been reported to retain binding affinity for GRPR (e.g. see: Lau, et al., 2019, ACS Omega 4:1470-1478). D-Cpa, Tpi, D-Tpi and D-Nal at position Xaa¹ have been reported to retain strong binding affinity for GRPR (e.g. see: Tables 1 and 3 in Cai et al., 1994 Proc. Natl. Acad. Sci. USA 91:12664-12668; RC-3965-II disclosed in Reile et al., 1995 International Journal of Oncology 7:749-754). Since both L-Tpi and D-Tpi retain binding affinity, the L-isomers of D-Nal and D-Cpa would also retain strong binding affinity for GRPR.

In some embodiments, Xaa² is Asn. In other embodiments, Xaa² is Gln. In other embodiments, Xaa² is Hse. In other embodiments, Xaa² is Cit. In other embodiments, Xaa² is His. Gln at position Xaa² is found in wildtype BBN. His at position Xaa² is found in wildtype GRP. Hse and Cit at position Xaa² have been reported to retain binding affinity for GRPR (see Gunther, et al., 2021, J Nucl Med. 62 (supplement 1) 1474).

In some embodiments, Xaa³ is Trp. In other embodiments, Xaa³ is Bta. In other embodiments, Xaa³ is αMe-Trp. In other embodiments, Xaa³ is Trp(Me). In other embodiments, Xaa³ is Trp(7-Me). In other embodiments, Xaa³ is Trp(6-Me). In other embodiments, Xaa³ is Trp(5-Me). In other embodiments, Xaa³ is Trp(4-Me). In other embodiments, Xaa³ is Trp(2-Me). In other embodiments, Xaa³ is Trp(7-F). In other embodiments, Xaa³ is Trp(6-F). In other embodiments, Xaa³ is Trp(5-F). In other embodiments, Xaa³ is Trp(4-F). In other embodiments, Xaa³ is Trp(5-OH). Trp at position Xaa³ is found in wildtype BBN and GRP. Bta and αMe-Trp at position Xaa³ have been reported to retain binding affinity for GRPR (see Gunther, et al., 2021, Journal of Nuclear Medicine 62 (supplement 1) 1474; Gunther, et al., J Nucl Med. 2022, jnumed.121.263323; DOI: https://doi.org/10.2967/jnumed.121.263323).

In some embodiments, Xaa⁴ is Ala. In other embodiments, Xaa⁴ is Ser.

In some embodiments, Xaa⁵ is Val. In other embodiments, Xaa⁵ is Cpg. In other embodiments, Xaa⁵ is Tle. Val in position Xaa⁵ is found in wildtype BBN and GRP.

In some embodiments, Xaa⁶ is Gly. In other embodiments, Xaa⁶ is N-methyl-Gly. In other embodiments, Xaa⁶ is D-Ala. N-methyl-Gly and D-Ala at position Xaa⁶ have been reported to retain strong binding affinity for GRPR (e.g. see: Table 4 in Horwell et al., 1996 Int. J. Peptide Protein Res. 48:522-531; Table 3 in Lin et al., 1995 European Journal of Pharmacology 284:55-69).

In some embodiments, Xaa⁷ is His. In other embodiments, Xaa⁷ is NMe-His. His at position Xaa⁷ is found in wildtype BBN and GRP. NMe-His at position Xaa7 has been reported to retain binding affinity for GRPR (e.g. see: Table 4 in Horwell et al., 1996 Int. J. Peptide Protein Res. 48:522-531).

In some embodiments, Xaa⁸ is Leu. In other embodiments, Xaa⁸ is D-Pro. In other embodiments, Xaa⁸ is Phe. Leu at position Xaa⁸ is found in wildtype BBN and GRP. D-Pro at position Xaa⁸ has been reported to retain binding affinity for GRPR (e.g. see: Leban, et al., 1994, J. Med. Chem. 37:439-445). Phe at position Xaa⁸ is supported by Phe at this position in ranatensin and litorin, which have very strong binding affinity to the GRPR (Heimbrook et al., 1991 J. Med. Chem. 34:2102-2107; Lin et al., 1995 European Journal of Pharmacology 294:55-69).

In some embodiments, Xaa⁹ is Pro (i.e. Xaa⁹-NH₂ is C-terminally amidated Pro). In other embodiments, Xaa⁹ is Phe (i.e. Xaa⁹-NH₂ is C-terminally amidated Phe). In other embodiments, Xaa⁹ is 4-oxa-L-Pro (i.e. Xaa⁹-NH₂ is C-terminally amidated 4-oxa-L-Pro). In other embodiments, Xaa⁹ is Me₂Thz (i.e. Xaa⁹-NH₂ is C-terminally amidated Me₂Thz). In other embodiments, Xaa⁹ is Thz (i.e. Xaa⁹-NH₂ is C-terminally amidated Thz). Pro at position Xaa⁹ has been reported to retain binding affinity for GRPR (e.g. see: Lau, et al., 2019, ACS Omega 4:1470-1478; WO/2021/068051). Phe at position Xaa⁹ has been reported to retain binding affinity for GRPR (e.g. see: Leban, et al., 1994, J. Med. Chem. 37:439-445). Thz at position Xaa⁹ has been reported to retain binding affinity for GRPR (e.g. see: Cai, et al., 1994 Proc Natl Acad Sci USA 91:12664-12668).

In some embodiments, “ψ” represents a peptide bond joining Xaa⁸ and Xaa⁹. In other embodiments, “ψ” represents a reduced peptide bond joining Xaa⁸ and Xaa⁹, meaning that the main chain amide (e.g. —C(O)NH—) formed between Xaa⁸ and Xaa⁹ is replaced by

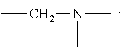

In some embodiments, -Xaa¹-Xaa²-Xaa³-Xaa⁴-Xaa⁵-Xaa⁶-Xaa⁷-Xaa⁸-ψ-Xaa⁹-NH₂ is -D-Phe-Gln-Trp-Ala-Val-Gly-His-Leu-Thz-NH₂.

In some embodiments, -Xaa¹-Xaa²-Xaa³-Xaa⁴-Xaa⁵-Xaa⁶-Xaa⁷-Xaa⁸-ψ-Xaa⁹-NH₂ is -D-2-Nal-Gln-Trp-Ala-Val-Gly-His-Leu-Thz-NH₂.

In some embodiments, -Xaa¹-Xaa²-Xaa³-Xaa⁴-Xaa⁵-Xaa⁶-Xaa⁷-Xaa⁸-ψ-Xaa⁹-NH₂ is -D-Phe-Gln-Trp-Ala-Val-Gly-NMe-His-Leu-Thz-NH₂.

In some embodiments, -Xaa¹-Xaa²-Xaa³-Xaa⁴-Xaa⁵-Xaa⁶-Xaa⁷-Xaa⁸-ψ-Xaa⁹-NH₂ is -D-Phe-Gln-Trp-Ala-Tle-Gly-His-Leu-Thz-NH₂.

In some embodiments, -Xaa¹-Xaa²-Xaa³-Xaa⁴-Xaa⁵-Xaa⁶-Xaa⁷-Xaa⁸-ψ-Xaa⁹-NH₂ is -Phe-Gln-Trp-Ala-Tle-Gly-NMe-His-Leu-Thz-NH₂.

In some embodiments, -Xaa¹-Xaa²-Xaa³-Xaa⁴-Xaa⁵-Xaa⁶-Xaa⁷-Xaa⁸-ψ-Xaa⁹-NH₂ is -D-Phe-His-Trp-Ala-Val-Gly-His-LeuψThz-NH₂.

In some embodiments, -Xaa¹-Xaa²-Xaa³-Xaa⁴-Xaa⁵-Xaa⁶-Xaa⁷-Xaa⁸-ψ-Xaa⁹-NH₂ is -D-Phe-His-Trp-Ala-Tle-Gly-NMe-His-Leu-Thz-NH₂.

In some embodiments, -Xaa¹-Xaa²-Xaa³-Xaa⁴-Xaa⁵-Xaa⁶-Xaa⁷-Xaa⁸-ψ-Xaa⁹-NH₂ is -D-Phe-Gln-Trp-Ala-Tle-Gly-His-LeuψThz-NH₂.

R^{L} is a linkage moiety joining the linker to the N-terminus of Xaa¹. In some embodiments, R^{L} is —C(O)—. In other embodiments, R^{L} is —NH—C(O)—. In yet other embodiments, R^{L} is —NH—C(S)—;

The linker enables attachment of 1-5 radiolabelling groups, and optionally an albumin binder, to the compound.

A non-limiting example of a suitable linker is a peptide linker. More generally, the linker is a linear or branched chain of n1 units of -L¹R¹— and/or -(L¹)₂R¹—(i.e. each unit is independently -L¹R¹— or -(L¹)₂R¹—), wherein n1 is 1-20. In alternative embodiments, n1 is 1, 2, 3, 4, 5, 6, 7, 8, 9, 10, 11, 12, 13, 14, 15, 16, 17, 18, 19, or 20. In some embodiments, n1 is 1-7. In some embodiments, n1 is 1. In other embodiments, n1 is 2. In other embodiments, n1 is 3. In other embodiments, n1 is 4. In other embodiments, n1 is 5. In other embodiments, n1 is 6. In other embodiments, n1 is 7.

In some embodiments, n6 is 1 and n1 is 1.

In some embodiments, n6 is 1, n1 is 1, and L¹ is —C(O)NH—.

In some embodiments, n6 is 1, n1 is 1, L¹ is —C(O)NH—, and R^{L} is —C(O)—.

In some embodiments, n6 is 1, n1 is 1, L¹ is —C(O)NH—, R^{L} is —C(O)—, and R¹ is a linear C₁₋₅ alkylenyl or —(CH₂)₂—[O(CH₂)₂]₁₋₆—(CH₂)₀₋₂.

In some embodiments, R^{rad}ₙ₆-[linker]- is configured as shown in Formula II:

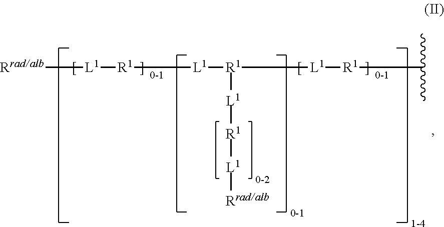

wherein L¹ and R¹ are as defined in the definition of the linker in Formula I, and R^{rad/alb} is either R^{rad} or R^{alb}, and wherein 0-1 R^{rad/alb} is R^{alb}.

Each R¹ (Formula I or II) is, independently, a linear, branched, and/or cyclic Cₙ₂ alkylenyl, alkenylenyl and/or alkynylenyl, wherein each n2 is independently 1-20, wherein any carbon bonded to two other carbons is optionally independently replaced by N, S, or O, and carbons are optionally independently substituted. In some embodiments, each n2 is independently 1-15 or 1-10. In alternative embodiments, each n2 is independently 1, 2, 3, 4, 5, 6, 7, 8, 9, 10, 11, 12, 13, 14, 15, 16, 17, 18, 19, or 20. In some embodiments, each R¹ is independently a Cₙ₂ alkylenyl wherein any carbon bonded to two other carbons is optionally independently replaced by N, S, or O, and carbons are optionally independently substituted. In some embodiments, each R¹ is independently a linear C₁₋₅ alkylenyl or —(CH₂)₂—[O(CH₂)₂]₁₋₆—(CH₂)₀₋₂—; in some of these embodiments, n1 is 1-7. In some embodiments, each R¹ is independently —C(R^{aa})H—, wherein each R^{aa} is independently the sidechain of a proteinogenic amino acid or the sidechain of an alpha amino acid from Table 1. In some embodiments, each R¹ is independently a proteinogenic amino acid or an amino acid from Table 1 omitting the backbone amino and carboxylic acid groups of the amino acid.

Each L¹ (Formula I or II) is a linkage group. In some embodiments, at least one L¹ is —S—. In some embodiments, at least one L¹ is —N(R²)C(O)—; in some of these embodiments, at least one R² is hydrogen. In some embodiments, at least one L¹ is —C(O)N(R²)—; in some of these embodiments, at least one R² is hydrogen. In some embodiments, at least one L¹ is —NH—C(O)—NH—. In some embodiments, at least one L¹ is —NH—C(S)—NH—. In some embodiments, at least one L¹ is

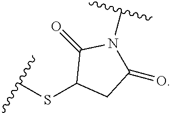 [SMILES: *SC1CC(=O)N(*)C1=O]

In some embodiments, at least one L¹ is

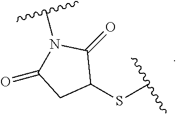 [SMILES: *SC1CC(=O)N(*)C1=O]

In some embodiments, at least one L¹ is

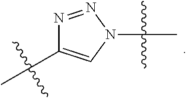 [SMILES: *c1cn(*)nn1]

In some embodiments, at least one L¹ is

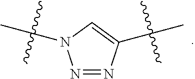 [SMILES: *c1cn(*)nn1]

In some embodiments, the linker has the configuration shown in Formula II, and each R¹ is independently a linear C₁₋₅ alkylenyl or —(CH₂)₂—[O(CH₂)₂]₁₋₆—(CH₂)₀₋₂—.

In some embodiments, n6 is 1, the linker is L¹R¹ and together with R^{L} forms —C(O)-Xaa¹¹- wherein Xaa¹¹ is a proteinogenic amino acid residue or an amino acid residue selected from Table 1. In some embodiments, Xaa¹¹ is pABzA-DIG. In other embodiments, Xaa¹¹ is Pip. In other embodiments, Xaa¹¹ is dPEG2. In other embodiments, Xaa¹¹ is Acp.

In some embodiments, the linker together with R^{L} forms a peptide linker, wherein peptide (amide) bonds are independently optionally methylated, optionally replacing one or more amide bonds with 1,2,3-triazole linkages (product of a reaction between an azide and an alkyne). In some embodiments, the peptide linker is a linear peptide linker, optionally replacing one or more amide bonds with 1,2,3-triazole linkages. In some embodiments, the peptide linker is a branched peptide linker, where the amino acid residues may be connected through a combination of main chain amide (peptide) bonds and ‘side chain’-to-‘main chain’ or ‘side chain’-to-‘side chain’ bonds. For example, a branched peptide may be connected by one or more of: backbone (main chain) peptide (amide) bonds, ‘main chain’-to-side chain amide bonds (between an amino group and a carboxylic acid group), optionally replacing one or more amide bonds with 1,2,3-triazole linkages. In some such embodiments, the peptide linker is (Xaa¹⁰)₁₋₂₀, wherein each Xaa¹⁰ is independently a proteinogenic amino acid residue or a non-proteinogenic amino acid residue (e.g. selected from Table 1) linked together as a linear or branched peptide linker. In some embodiments, (Xaa¹⁰)₁₋₂₀ is a linear peptide linker. In some embodiments, (Xaa¹⁰)₁₋₂₀ is a branched peptide linker. R^{rad} is bonded to the peptide linker through an amide bond or another L¹ linkage group; in some embodiments, R^{rad} is bonded to the peptide linker through an amide bond.

In some embodiments, each Xaa¹⁰ is independently —N(R^{a})R^{b}C(O)— wherein: R^{a} may be H or methyl; R^{b} may be a 1- to 30-atom alkylenyl, heterolakylenyl, alkenylenyl, heteroalkenylenyl, alkynylenyl, or heteroalkynylenyl, including linear, branched, and/or cyclic (whether aromatic or nonaromatic as well as mono-cyclic, multicyclic or fused cyclic) structures; or N, R^{a} and R^{b} together may form a 5- to 7-atom heteroalkylenyl or heteroalkenylenyl.

In some embodiments, (Xaa¹⁰)₁₋₂₀ consists of a single amino acid or residue. In some embodiments, (Xaa¹⁰)₁₋₂₀ is a dipeptide, wherein each Xaa¹⁰ may be the same or different. In some embodiments, (Xaa¹⁰)₁₋₂₀ is a tripeptide, wherein each Xaa¹⁰ may be the same, different or a combination thereof. In some embodiments, (Xaa¹⁰)₁₋₂₀ consists of 4 amino acid residues connected by peptide bonds, wherein each Xaa¹⁰ may be the same, different or a combination thereof. In some embodiments, each Xaa¹⁰ is independently selected from proteinogenic amino acids and the non-proteinogenic amino acids listed in Table 1, wherein each peptide backbone amino group of the peptide linker is independently optionally methylated. In some embodiments, all peptide backbone amino groups of the peptide linker are methylated. In other embodiments, only one peptide backbone amino group of the peptide linker is methylated. In other embodiments, only two peptide backbone amino groups of the peptide linker are methylated. In other embodiments, no peptide backbone amino groups of the peptide linker are methylated.

In some embodiments, n6 is 1. In other embodiments, n6 is 2. In other embodiments, n6 is 3. In other embodiments, n6 is 4. In other embodiments, n6 is 5.

In some embodiments, the linker does not comprise R^{alb}.

In some embodiments, the linker comprises R^{alb} bonded to an L¹ of the linker.

In some embodiments, R^{alb} is —(CH₂)ₙ₃—CH₃ wherein n3 is 8-20. In alternative embodiments, n3 is 8, 9, 10, 11, 12, 13, 14, 15, 16, 17, 18, 19, or 20.

In some embodiments, R^{alb} is —(CH₂)ₙ₄—C(O)OH wherein n4 is 8-20. In alternative embodiments, n4 is 8, 9, 10, 11, 12, 13, 14, 15, 16, 17, 18, 19, or 20.

In some embodiments, R^{alb} is

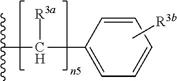

wherein n5 is 1-4 and R^{3a} is H or methyl, and R^{3b} is I, Br, F, Cl, H, OH, OCH₃, NH₂, NO₂ or C₁-C₆ alkyl. In alternative embodiments, n5 is 1, 2, 3, or 4. In certain embodiments, R^{3a} is H. In certain embodiments, R^{3a} is methyl. In certain embodiments, R^{3b} is I, Br, F, or Cl, optionally in para position. In certain embodiments, R^{3b} is H. In certain embodiments, R^{3b} is OH, optionally in para position. In certain embodiments, R^{3b} is OCH₃, optionally in para position. In certain embodiments, R^{3b} is NH₂, optionally in para position. In certain embodiments, R^{3b} is NO₂, optionally in para position. In certain embodiments, R^{3b} is C₁-C₆ alkyl, optionally in para position. In certain embodiments, R^{3a} is H and R^{3b} is OCH₃ or NO₂. In some embodiments, R^{3a} is methyl and R^{3b} is isobutyl, optionally para-isobutyl.

In some embodiments, R^{alb} is

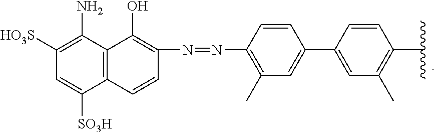 [SMILES: *c1ccc(-c2ccc(N=Nc3ccc4c(S(=O)(=O)O)cc(S(=O)(=O)O)c(N)c4c3O)c(C)c2)cc1C]

In some embodiments, at least one R^{rad} is or comprises a radiometal chelator. The radiometal chelator may be any chelator suitable for binding a radiometal, a radionuclide-bound metal, or a radionuclide-bound metal-containing prosthetic group, and which is attached to the linker by forming an amide bond (between an amino group and a carboxylic acid group) or a 1,2,3-triazole (reaction between an azide and an alkyne), or by reaction between a maleimide and a thiol group. Many suitable radiometal chelators are known, e.g. as summarized in Price and Orvig, Chem. Soc. Rev., 2014, 43, 260-290. In some embodiments, but without limitation, each radiometal chelator is independently selected from the group consisting of: DOTA and DOTA derivatives; DOTAGA; NOTA; NODAGA; NODASA; CB-DO2A; 3p-C-DEPA; TCMC; DO3A; DTPA and DTPA analogues optionally selected from CHX-A″-DTPA and 1B4M-DTPA; TETA; NOPO; Me-3,2-HOPO; CB-TE1A1P; CB-TE2P; MM-TE2A; DM-TE2A; sarcophagine and sarcophagine derivatives optionally selected from SarAr, SarAr-NCS, diamSar, AmBaSar, and BaBaSar; TRAP; AAZTA; DATA and DATA derivatives; H2-macropa or a derivative thereof; H₂dedpa, H₄octapa, H₄py4pa, H₄Pypa, H₂azapa, Hsdecapa, and other picolinic acid derivatives; CP256; PCTA; C-NETA; C-NE3TA; HBED; SHBED; BCPA; CP256; YM103; desferrioxamine (DFO) and DFO derivatives; H₆phospa; a trithiol chelate; mercaptoacetyl; hydrazinonicotinamide; dimercaptosuccinic acid; 1,2-ethylenediylbis-L-cysteine diethyl ester; methylenediphosphonate; hexamethylpropyleneamineoxime; and hexakis(methoxy isobutyl isonitrile). In some embodiments, at least one radiometal chelator is DOTA or a DOTA derivative.

Exemplary non-limiting examples of radiometal chelators and example radionuclides that may be chelated by these chelators are shown in Table 2. In alternative embodiments, at least one R^{rad} is a radiometal chelator selected from those listed above or in Table 2. It is noted, however, that one skilled in the art could replace any of the chelators listed herein with another chelator.

TABLE 2
Exemplary chelators and exemplary radionuclide which bind said chelators

| Chelator | Radionuclide |
|---|---|
| 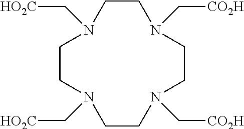 [SMILES: O=C(O)CN1CCN(CC(=O)O)CCN(CC(=O)O)CCN(CC(=O)O)CC1] | Cu-64/67 Ga-67/68 In-111 Lu-177 Y-86/90 Bi-203/212/213 Pb-212 Ac-225 |
| DOTA, 1,4,7,10-tetraazacyclododecane- | Gd-159 |
| 1,4,7,10-tetraacetic acid | Yb-175 |
| | Ho-166 |
| | As-211 |
| | Sc-44/47 |
| | Pm-149 |
| | Pr-142 |
| | Sn-117m |
| | Sm-153 |
| | Tb-149/152/155/161 |
| | Er-165 |
| | Ra-223/224 |
| | Th-227 |
| 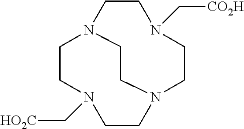 [SMILES: O=C(O)CN1CCN2CCN(CC1)CCN(CC(=O)O)CC2] | Cu-64/67 |
| CB-DO2A, 4,10-bis(carboxymethyl)-1,4,7,10- | |
| tetraazabicyclo[5.5.2]tetradecane | |
| 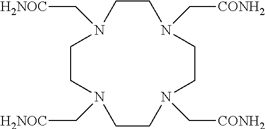 [SMILES: NC(=O)CN1CCN(CC(N)=O)CCN(CC(N)=O)CCN(CC(N)=O)CC1] | Pb-212 |
| TCMC, 1,4,7,10-tetrakis(carbamoylmethyl)- | |
| 1,4,7,10-tetraazacyclododecane | |
| 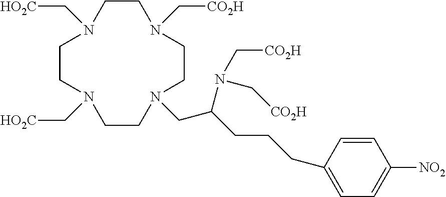 [SMILES: O=C(O)CN1CCN(CC(=O)O)CCN(CC(CCCc2ccc([N+](=O)[O-])cc2)N(CC(=O)O)CC(=O)O)CCN(CC(=O)O)CC1] | Bi-212/213 |
| 3p-C-DEPA | |
| 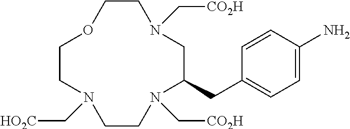 [SMILES: Nc1ccc(C[C@@H]2CN(CC(=O)O)CCOCCN(CC(=O)O)CCN2CC(=O)O)cc1] | Cu-64/67 |
| p-NH₂-Bn-Oxo-DO3A | |
| 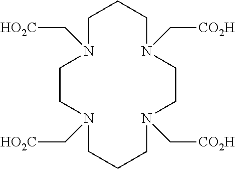 [SMILES: O=C(O)CN1CCCN(CC(=O)O)CCN(CC(=O)O)CCCN(CC(=O)O)CC1] | Cu-64/67 |
| TETA, 1,4,8,11-tetraazacyclotetradecane- | |
| 1,4,8,11-tetraacetic acid | |
| 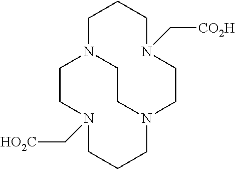 [SMILES: O=C(O)CN1CCCN2CCN(CCCN(CC(=O)O)CC2)CC1] | Cu-64/67 |
| CB-TE2A, 4,11-bis- | |
| (carboxymethyl)-1,4,8,11- | |
| tetraazabicyclo[6.6.2]- | |
| hexadecane | |
| 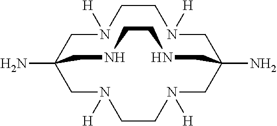 | Cu-64/67 |
| Diamsar | |
| 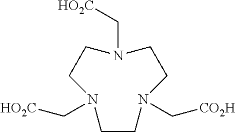 [SMILES: O=C(O)CN1CCN(CC(=O)O)CCN(CC(=O)O)CC1] | Cu-64/67 Ga-68 In-111 Sc-44/47 |
| NOTA, 1,4,7-triazacyclononane-1,4,7- | |
| triacetic acid | |
| 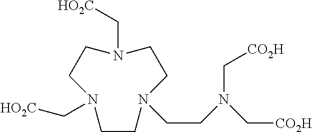 [SMILES: O=C(O)CN1CCN(CCN(CC(=O)O)CC(=O)O)CCN(CC(=O)O)CC1] | Cu-64/67 Ga-68 Lu-177 Y-86/90 Bi-213 Pb-212 |
| NETA, {4-[2-(bis-carboxymethylamino)-ethyl]- | |
| 7-carboxymethyl-[1,4,7]triazonan-1-yl}-acetic acid | |
| 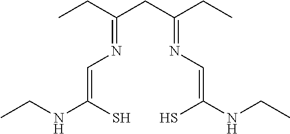 [SMILES: CCN/C(S)=C/N=C(\CC)C/C(CC)=N/C=C(\S)NCC] | Au-198/199 |
| HxTSE | |
| 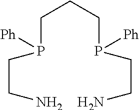 [SMILES: NCCP(CCCP(CCN)c1ccccc1)c1ccccc1] | Rh-105 |
| P₂N₂Ph₂ | |
| 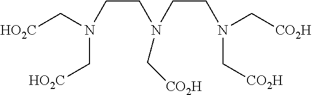 [SMILES: O=C(O)CN(CCN(CC(=O)O)CC(=O)O)CCN(CC(=O)O)CC(=O)O] | In-111 Sc-44/47 Lu-177 Y-86/90 Sn-117m Pd-109 |
| DTPA, diethylenetriaminepentaacetic acid | |
| 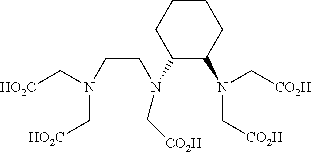 | In-111 Lu-177 Y-86/90 Bi-212/213 |
| CHX-A00-DTPA, 2-(p-isothiocyanatobenzyl)- | |
| cyclohexyldiethylenetriaminepentaacetic | |
| acid | |
| 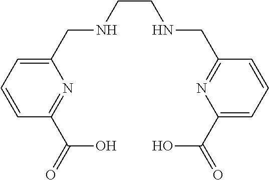 [SMILES: O=C(O)c1cccc(CNCCNCc2cccc(C(=O)O)n2)n1] | Cu-64/67 |
| H₂dedpa, 1,2-[[6-(carboxy)-pyridin-2-yl]- | |
| methylamino]ethane | |
| 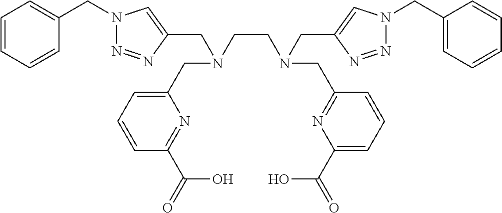 [SMILES: O=C(O)c1cccc(CN(CCN(Cc2cn(Cc3ccccc3)nn2)Cc2cccc(C(=O)O)n2)Cc2cn(Cc3ccccc3)nn2)n1] | Cu-64/67 |
| H₂azapa, N,N0-[1-benzyl-1,2,3-triazole-4-yl]methyl- | |
| N,N0-[6-(carboxy)pyridin-2-yl]-1,2-diaminoethane | |
| 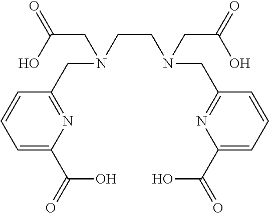 [SMILES: O=C(O)CN(CCN(CC(=O)O)Cc1cccc(C(=O)O)n1)Cc1cccc(C(=O)O)n1] | In-111 Lu-177 Y-86/90 Ac-225 |
| H₄octapa | |
| 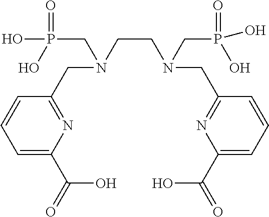 [SMILES: O=C(O)c1cccc(CN(CCN(Cc2cccc(C(=O)O)n2)CP(=O)(O)O)CP(=O)(O)O)n1] | Ac-225 |
| H₆phospa | |
| 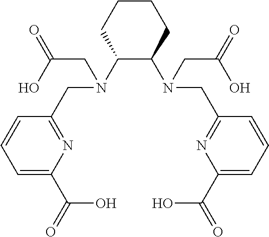 [SMILES: O=C(O)CN(Cc1cccc(C(=O)O)n1)[C@@H]1CCCC[C@H]1N(CC(=O)O)Cc1cccc(C(=O)O)n1] | In-111 Ac-225 |
| H₄CHXoctapa | |
| 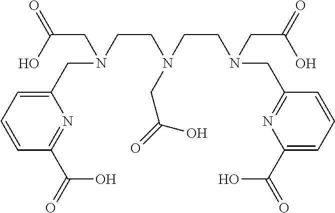 [SMILES: O=C(O)CN(CCN(CC(=O)O)Cc1cccc(C(=O)O)n1)CCN(CC(=O)O)Cc1cccc(C(=O)O)n1] | In-111 Lu-177 Ac-225 |
| H₅decapa | |
| 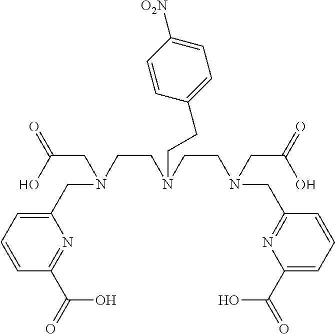 [SMILES: O=C(O)CN(CCN(CCc1ccc([N+](=O)[O-])cc1)CCN(CC(=O)O)Cc1cccc(C(=O)O)n1)Cc1cccc(C(=O)O)n1] | In-111 Lu-177 Ac-225 |
| H₄neunpa-p-Bn-NO₂ | |
| 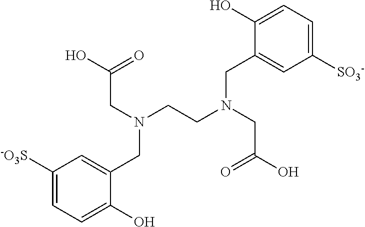 | In-111 Ga-68 |
| SHBED, N,N0-bis(2-hydroxy-5-sulfobenzyl)- | |
| ethylenediamine-N,N0-diacetic acid | |
| 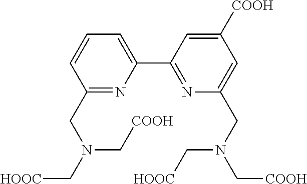 [SMILES: O=C(O)CN(CC(=O)O)Cc1cccc(-c2cc(C(=O)O)cc(CN(CC(=O)O)CC(=O)O)n2)n1] | In-111 |
| BPCA | |
| 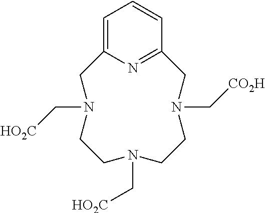 [SMILES: O=C(O)CN1CCN(CC(=O)O)Cc2cccc(n2)CN(CC(=O)O)CC1] | Cu-64/67 |
| PCTA, 3,6,9,15-tetraazabicyclo[9.3.1]- | |
| pentadeca-1(15),11,13-triene-3,6,9,- | |
| triacetic acid | |
| 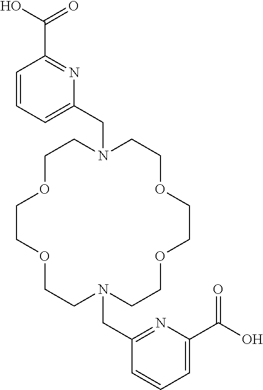 [SMILES: O=C(O)c1cccc(CN2CCOCCOCCN(Cc3cccc(C(=O)O)n3)CCOCCOCC2)n1] | Ac-225 |
| H2-MACROPA | |
| (N,N′-bis[(6-carboxy-2-pyridil)methyl]-4,13-diaza-18-crown-6) | |
| 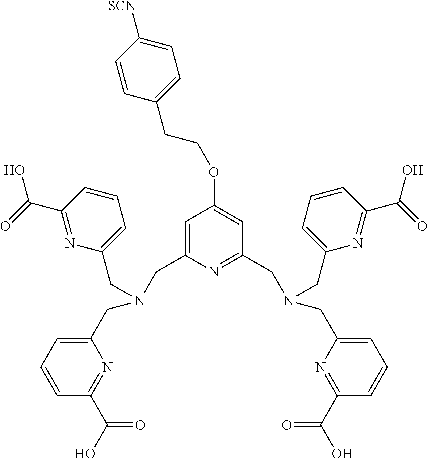 | Ac-225 |
| H4py4pa-phenyl-NCS | |
| 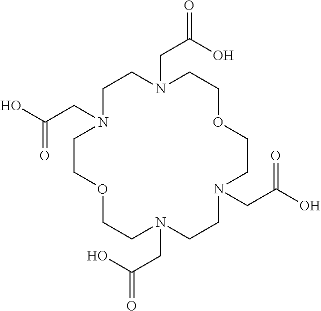 [SMILES: O=C(O)CN1CCOCCN(CC(=O)O)CCN(CC(=O)O)CCOCCN(CC(=O)O)CC1] | Ac-225 |
| Crown | |
| 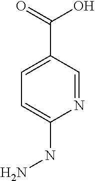 [SMILES: NNc1ccc(C(=O)O)cn1] | Tc-99m Tc-94m |
| HYNIC | |
| 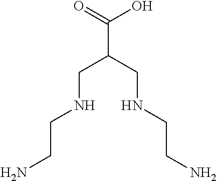 [SMILES: NCCNCC(CNCCN)C(=O)O] | Tc-99m Tc-94m |
| N4 (6-carboxy-1,4,7,11-tetraazaundecane) | |
| 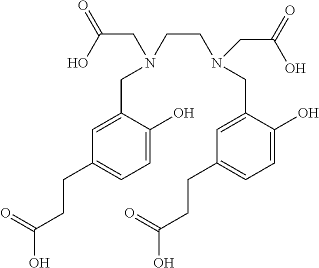 [SMILES: O=C(O)CCc1ccc(O)c(CN(CCN(CC(=O)O)Cc2cc(CCC(=O)O)ccc2O)CC(=O)O)c1] | Ga-68 |
| HBED-CC | |

In some embodiments, each radiometal chelator is independently selected from Table 2, wherein each chelator is optionally bound by a radiometal. In some embodiments, each radiometal chelator is bound by one of the corresponding radionuclides shown in Table 2.

In some embodiments, at least one R^{rad} is DOTA, or a derivative thereof, linked via an amide (e.g. formed from one of the carboxyl groups shown in Table 2. In some embodiments, at least one R^{rad} is CB-DO2A, or a derivative thereof, linked via an amide (e.g. formed from one of the carboxyl groups shown in Table 2). In some embodiments, at least one R^{rad} is TCMC, or a derivative thereof, linked via an amide (e.g. formed from one of the —CONH₂ groups shown in Table 2). In some embodiments, the chelator at least one R^{rad} is 3p-C-DEPA, or a derivative thereof, linked via an amide (e.g. formed from one of the carboxyl groups shown in Table 2). In some embodiments, at least one R^{rad} is p-NH₂-Bn-Oxo-DO3A or a derivative thereof, linked via an amide (e.g. formed from one of the carboxyl groups shown in Table 2). In some embodiments, at least one R^{rad} is TETA, or a derivative thereof, linked via an amide (e.g. formed from one of the carboxyl groups shown in Table 2). In some embodiments, at least one R^{rad} is CB-TE2A, or a derivative thereof, linked via an amide (e.g. formed from one of the carboxyl groups shown in Table 2). In some embodiments, at least one R^{rad} is Diamsar, or a derivative thereof, linked via an amide (e.g. formed from one of the amino groups shown in Table 2). In some embodiments, at least one R^{rad} is NOTA, or a derivative thereof, linked via an amide (e.g. formed from one of the carboxyl groups shown in Table 2). In some embodiments, at least one R^{rad} is NETA, or a derivative thereof, linked via an amide (e.g. formed from one of the carboxyl groups shown in Table 2). In some embodiments, at least one R^{rad} is HxTSE, or a derivative thereof, linked via an amide (e.g. formed from one of the amino groups shown in Table 2). In some embodiments, at least one R^{rad} is P₂N₂Ph₂, or a derivative thereof, linked via an amide (e.g. formed from one of the amino groups shown in Table 2). In some embodiments, at least one R^{rad} is DTPA, or a derivative thereof, linked via an amide (e.g. formed from one of the carboxyl groups shown in Table 2). In some embodiments, at least one R^{rad} is CHX-A00-DTPA, or a derivative thereof, linked via an amide (e.g. formed from one of the carboxyl groups shown in Table 2). In some embodiments, at least one R^{rad} is H₂dedpa, or a derivative thereof, linked via an amide (e.g. formed from one of the carboxyl groups shown in Table 2). In some embodiments, at least one R^{rad} is H₂azapa, or a derivative thereof, linked via an amide (e.g. formed from one of the carboxyl groups shown in Table 2). In some embodiments, at least one R^{rad} is H₄octapa, or a derivative thereof, linked via an amide (e.g. formed from one of the carboxyl groups shown in Table 2). In some embodiments, at least one R^{rad} is Hephospa, or a derivative thereof, linked via an amide (e.g. formed from one of the carboxyl groups shown in Table 2). In some embodiments, at least one R^{rad} is H₄CHXoctapa, or a derivative thereof, linked via an amide (e.g. formed from one of the carboxyl groups shown in Table 2). In some embodiments, at least one R^{rad} is Hsdecapa, or a derivative thereof, linked via an amide (e.g. formed from one of the carboxyl groups shown in Table 2). In some embodiments, at least one R^{rad} is H₄neunpa-p-Bn-NO2, or a derivative thereof, linked via an amide (e.g. formed from one of the carboxyl groups shown in Table 2). In some embodiments, at least one R^{rad} is SHBED, or a derivative thereof, linked via an amide (e.g. formed from one of the carboxyl groups shown in Table 2). In some embodiments, at least one R^{rad} is BPCA, or a derivative thereof, linked via an amide (e.g. formed from one of the carboxyl groups shown in Table 2). In some embodiments, at least one R^{rad} is PCTA, or a derivative thereof, linked via an amide (e.g. formed from one of the carboxyl groups shown in Table 2). In some embodiments, at least one R^{rad} is H2-MACROPA, or a derivative thereof, linked via an amide (e.g. formed from one of the carboxyl groups shown in Table 2). In some embodiments, at least one R^{rad} is Crown, or a derivative thereof, linked via an amide (e.g. formed from one of the carboxyl groups shown in Table 2). In some embodiments, at least one R^{rad} is HYNIC, or a derivative thereof, linked via an amide (e.g. formed from the carboxyl group shown in Table 2). In some embodiments, at least one R^{rad} is N4, or a derivative thereof, linked via an amide (e.g. formed from the carboxyl group shown in Table 2). In some embodiments, at least one R^{rad} is HBED-CC, or a derivative thereof, linked via an amide (e.g. formed from one of the carboxyl groups shown in Table 2).

In some embodiments, the radiometal chelator (or one of the radiometal chelators) is a derivative of a radiometal chelator shown in Table 2. A derivative may include, e.g. (1) modification of a functional group of the chelator (e.g. a carboxyl group, an amino group, etc.) or (2) attachment of a new functional group (e.g. attachment of an R-group to an ethylene carbon located between two nitrogen atoms, wherein the R-group is a functional group fused to a spacer). In some embodiments, a carboxyl functional group shown in Table 2 is replaced with azidopropyl ethylacetamide (e.g. azido-mono-amide-DOTA), butynylacetamide (e.g. butyne-DOTA), thioethylacetamide (e.g. DO3A-thiol), maleimidoethylacetamide (e.g. maleimido-mono-amide-DOTA), or N-hydroxysuccinimide ester (e.g. DOTA-NHS-ester). When linked, these derivative chelators can be linked either via an amide (formed from a remaining carboxyl group) or via —C(O)—NH—(CH₂)₂₋₃-(triazole) or —C(O)—NH—(CH₂)₂₋₃-(thiomaleimide). In other embodiments, a backbone carbon (e.g. in an ethylene positioned between two backbone nitrogen atoms) in the chelator ring is fused to an R-group containing a functional group, optionally wherein the R-group is —(CH₂)₁₋₃-(phenyl)-N═C═S or —(CH₂)₁₋₃-(phenyl)-N═C═O, optionally 1,4-isothiocyanatobenzyl; e.g. p-SCN-Bn-DOTA (S-2-(4-isothiocyanatobenzyl)-1,4,7,10-tetraazacyclododecane tetraacetic acid), p-SCN-Bn-NOTA (2-S-(4-isothiocyanatobenzyl)-1,4,7-triazacyclononane-1,4,7-triacetic acid), and the like. When linked, these derivatives can form a urea linkage (formed from isocyanate) or a thiourea linkage (formed from isothiocyanate).

In some embodiments, a radiometal chelator is conjugated with a radiometal, a radionuclide-bound metal, or a radionuclide-bound metal-containing prosthetic group, and the radiometal, the radionuclide-bound metal, or the radionuclide-bound metal-containing prosthetic group is chelated to the radionuclide-chelator complex. In some embodiments, the radiometal, the radionuclide-bound metal, or the radionuclide-bound metal-containing prosthetic group is: ⁶⁸Ga, ⁶¹Cu, ⁶⁴Cu, ⁶⁷Cu, ⁶⁷Ga, ¹¹¹In, ⁴⁴Sc, ⁸⁶Y, ⁸⁹Zr, ⁹⁰Nb, ¹⁷⁷Lu, ^{117m}Sn, ¹⁶⁵Er, ⁹⁰Y, ²²⁷Th, ²²⁵Ac, ²¹³Bi, ²¹²Bi, ⁷²As, ⁷⁷As, ²¹¹At, ²⁰³Pb, ²¹²Pb, ⁴⁷Sc, ¹⁶⁶Ho, ¹⁸⁸Re, ¹⁸⁶Re, ¹⁴⁹Pm, ¹⁵⁹Gd, ¹⁰⁵Rh, ¹⁰⁹Pd, ¹⁹⁸Au, ¹⁹⁹Au, ¹⁷⁵Yb, ¹⁴²Pr, ^{114m}In, ^{94m}Tc, ^{99m}Tc, ¹⁴⁹Tb, ¹⁵²Tb, ¹⁵⁵Tb, ¹⁶¹Tb, or [¹⁸F]AlF. In other embodiments, the radiometal, the radionuclide-bound metal, or the radionuclide-bound metal-containing prosthetic group is: ⁶⁸Ga, ⁶¹Cu, ⁶⁴Cu, ⁶⁷Cu, ⁶⁷Ga, ¹¹¹In, ⁴⁴Sc, ⁸⁶Y, ¹⁷⁷Lu, ⁹⁰Y, ²²⁵Ac, ²¹³Bi, or ²¹²Bi. In some embodiments, the chelator is a chelator from Table 2 and the chelated radionuclide is a radionuclide indicated in Table 2 as a binder of the chelator.

In some embodiments, the chelator is: DOTA or a derivative thereof, conjugated with ¹⁷⁷Lu, ¹¹¹In, ²¹³Bi, ⁶⁸Ga, ⁶⁷Ga, ²⁰³Pb, ²¹²Pb, ⁴⁴Sc, ⁴⁷Sc, ⁹⁰Y, ⁸⁶Y, ²²⁵Ac, ^{117m}Sn, ¹⁵³Sm, ¹⁴⁹Tb, ¹⁵²Tb, ¹⁵⁵Tb, ¹⁶¹Tb, ¹⁶⁵Er, ²¹³Bi, ²²⁴Ra, ²¹²Bi, ²¹²Pb, ²²⁵Ac, ²²⁷Th, ²²³Ra, ⁴⁷Sc, ⁶⁴Cu or ⁶⁷Cu; H2-MACROPA conjugated with ²²⁵Ac; Me-3,2-HOPO conjugated with ²²⁷Th; H₄py4pa conjugated with ²²⁵Ac, ²²⁷Th or ¹⁷⁷Lu; H₄pypa conjugated with ¹⁷⁷Lu; NODAGA conjugated with ⁶⁸Ga; DTPA conjugated with ¹¹¹In; or DFO conjugated with ⁸⁹Zr.

In some embodiments, the chelator is TETA (1,4,8,11-tetraazacyclotetradecane-1,4,8,11-tetraacetic acid), SarAr (1-N-(4-Aminobenzyl)-3,6,10,13,16,19-hexaazabicyclo[6.6.6]-eicosane-1,8-diamine), NOTA (1,4,7-triazacyclononane-1,4,7-triacetic acid), TRAP (1,4,7-triazacyclononane-1,4,7-tris[methyl(2-carboxyethyl)phosphinic acid), HBED (N,N0-bis(2-hydroxybenzyl)-ethylenediamine-N,N0-diacetic acid), 2,3-HOPO (3-hydroxypyridin-2-one), PCTA (3,6,9,15-tetraazabicyclo[9.3.1]-pentadeca-1(15),11,13-triene-3,6,9-triacetic acid), DFO (desferrioxamine), DTPA (diethylenetriaminepentaacetic acid), OCTAPA (N,N0-bis(6-carboxy-2-pyridylmethyl)-ethylenediamine-N,N0-diacetic acid) or another picolinic acid derivative.

In some embodiments, an R^{rad} is a chelator for radiolabelling with ^{99m}Tc, ^{94m}Tc, ¹⁸⁶Re, or ¹⁸⁸Re, such as mercaptoacetyl, hydrazinonicotinamide, dimercaptosuccinic acid, 1,2-ethylenediylbis-L-cysteine diethyl ester, methylenediphosphonate, hexamethylpropyleneamineoxime and hexakis(methoxy isobutyl isonitrile), and the like. In some embodiments, an R^{rad} is a chelator, wherein the chelator is mercaptoacetyl, hydrazinonicotinamide, dimercaptosuccinic acid, 1,2-ethylenediylbis-L-cysteine diethyl ester, methylenediphosphonate, hexamethylpropyleneamineoxime or hexakis(methoxy isobutyl isonitrile). In some of these embodiments, the chelator is bound by a radionuclide. In some such embodiments, the radionuclide is ^{99m}Tc, ^{94m}Tc, ¹⁸⁶Re, or ¹⁸⁸Re.

In some embodiments, an R^{rad} is a chelator that can bind ¹⁸F-aluminum fluoride ([¹⁸F]AlF), such as 1,4,7-triazacyclononane-1,4-diacetate (NODA) and the like. In some embodiments, the chelator is NODA. In some embodiments, the chelator is bound by [¹⁸F]AlF.

In some embodiments, an R^{rad} is a chelator that can bind ⁷²As or ⁷⁷As, such as a trithiol chelate and the like. In some embodiments, the chelator is a trithiol chelate. In some embodiments, the chelator is conjugated to ⁷²As. In some embodiments, the chelator is conjugated to ⁷⁷As.

In certain embodiments, at least one R^{rad} is a prosthetic group containing a trifluoroborate (BF₃), capable of ¹⁸F/¹⁹F exchange radiolabeling. In some of these embodiments, the R^{rad} is BF₃—R⁵—R⁴—, wherein R⁴ is —(CH₂)₁₋₅, optionally methylene, and wherein BF₃—R⁵— forms

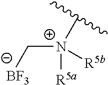

wherein R^{5a} and R^{5b} are each independently a C₁-C₅ linear or branched alkyl group, or a structure listed in Table 3 (below) or Table 4 (below). For Tables 3 and 4, each R group in each pyridine substituted with —OR, —SR, —NR—, —NHR or —NR₂ is independently a C₁-C₅ linear or branched alkyl. In some embodiments, at least one BF₃—R⁵— forms

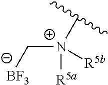

wherein R^{5a} and R^{5b} are each independently a C₁-C₅ linear or branched alkyl group. In some embodiments, at least one of the BF₃—R⁵— group(s) is/are selected from those listed in Table 3. In some embodiments, at least one of the BF₃—R⁵-group(s) is/are selected from those listed in Table 4. The trifluoroborate-containing prosthetic group(s) may comprise ¹⁸F. In some embodiments, one fluorine in BF₃ forms is ¹⁸F. In some embodiments, all three fluorines in BF₃ are ¹⁸F. In some embodiments, all three fluorines in BF₃ are ¹⁹F.

TABLE 3
Exemplary BF₃—R⁵— groups.

| | |
|---|---|
| 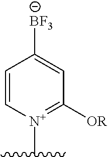 | 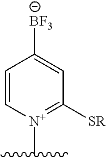 |
| 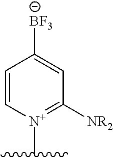 | 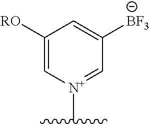 |
| 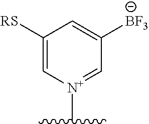 | 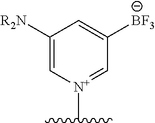 |
| 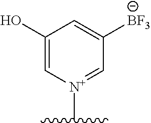 | 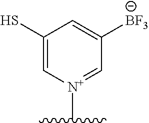 |
| 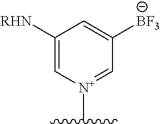 | 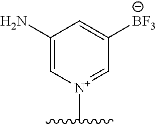 |
| 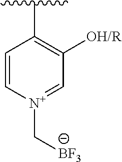 | 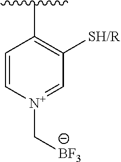 |
| 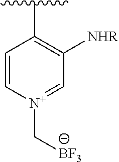 | 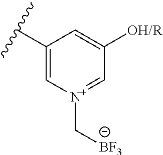 |
| 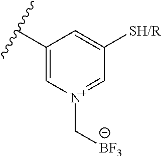 | 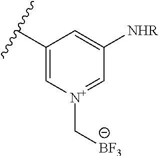 |
| 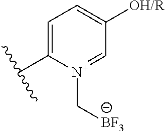 | 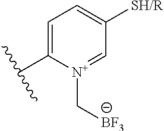 |
| 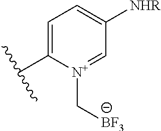 | 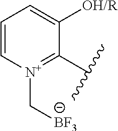 |
| 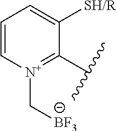 | 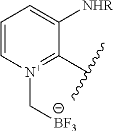 |
| 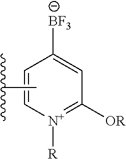 | 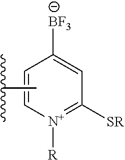 |
| 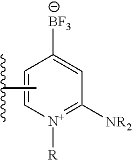 | 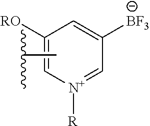 |
| 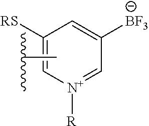 | 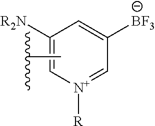 |
| 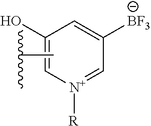 | 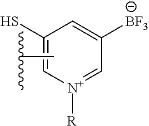 |
| 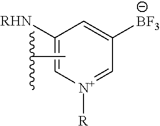 | 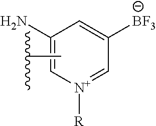 |
| 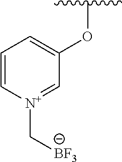 | 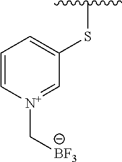 |
| 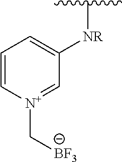 | 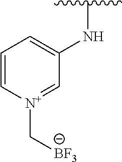 |
| 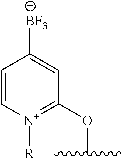 | 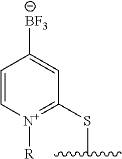 |
| 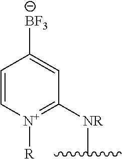 | 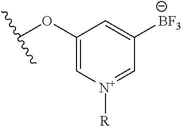 |
| 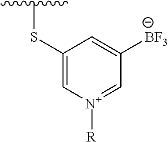 | 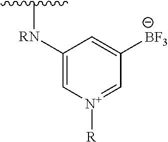 |
| 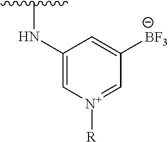 | 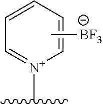 |

TABLE 4
Exemplary BF₃—R⁵— groups.

| | |
|---|---|
| 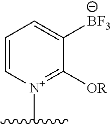 | 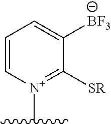 |
| 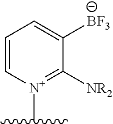 | 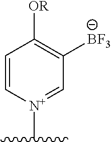 |
| 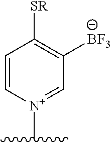 | 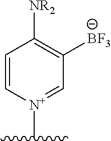 |
| 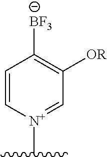 | 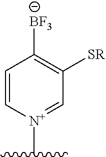 |
| 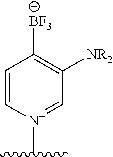 | 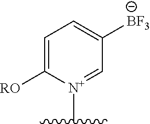 |
| 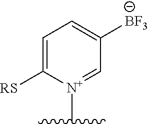 | 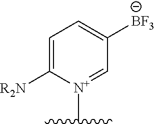 |
| 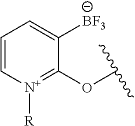 | 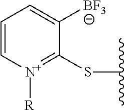 |
| 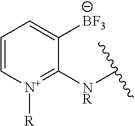 | 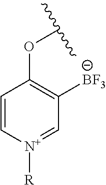 |
| 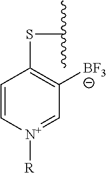 | 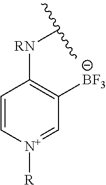 |
| 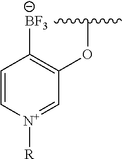 | 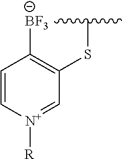 |
| 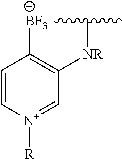 | 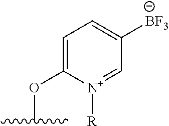 |
| 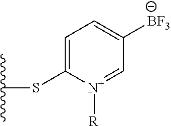 | 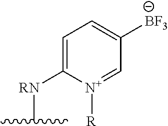 |
| 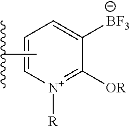 | 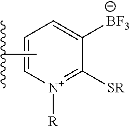 |
| 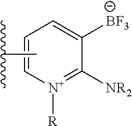 | 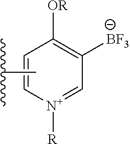 |
| 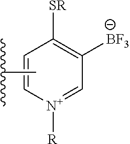 | 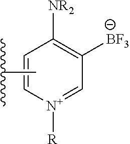 |
| 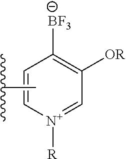 | 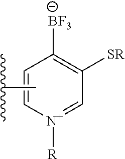 |
| 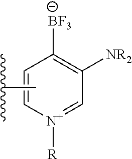 | 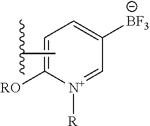 |
| 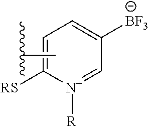 | 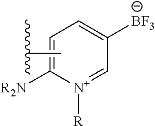 |
| 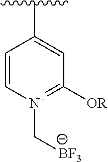 | 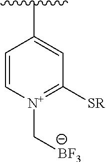 |
| 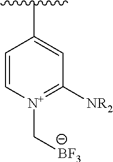 | 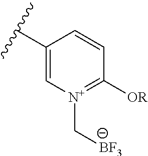 |
| 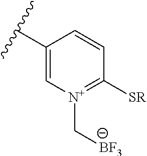 | 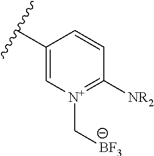 |
| 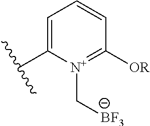 | 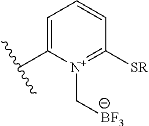 |
| 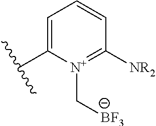 | 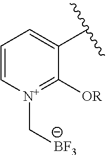 |
| 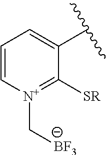 | 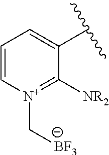 |
| 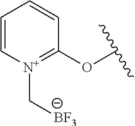 | 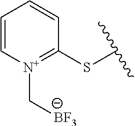 |
| 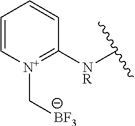 | 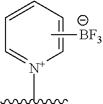 |

In some embodiments, a BF₃—R⁵— may independently form

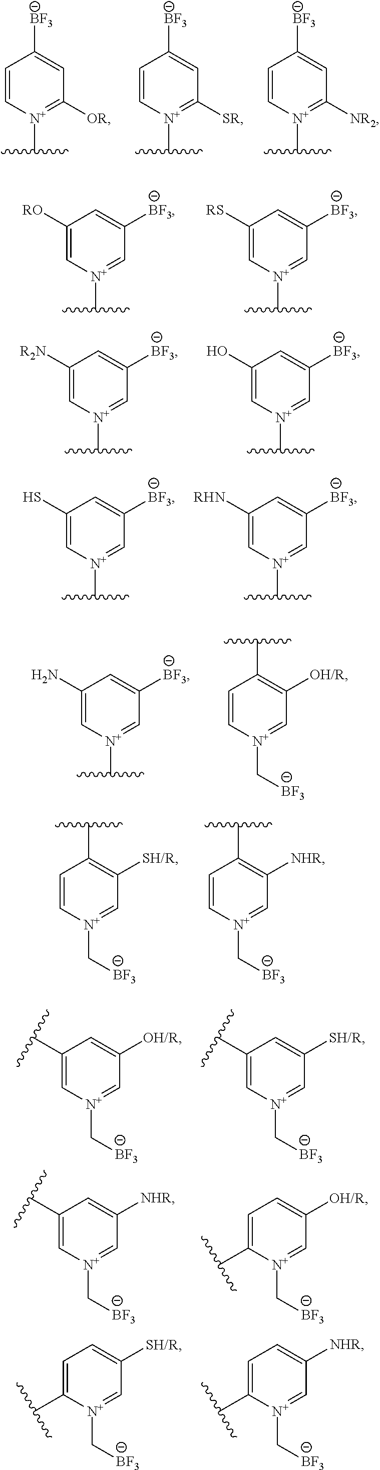

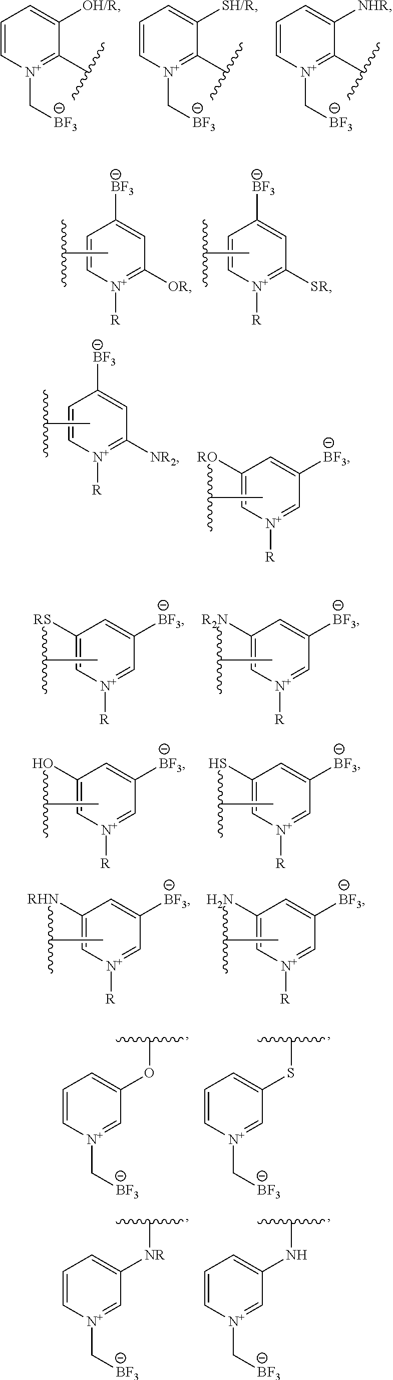

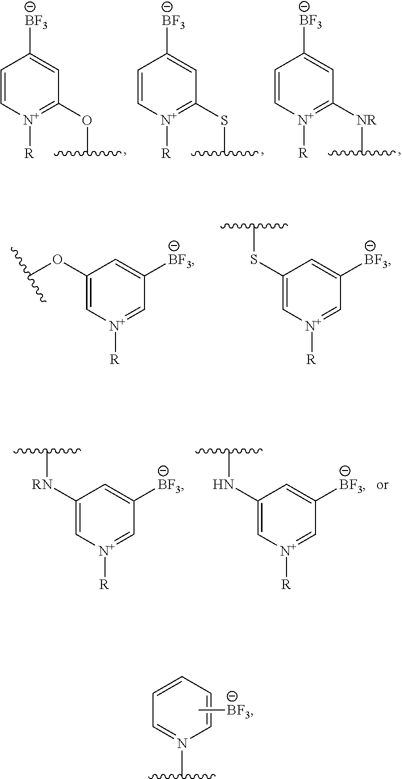

in which each R (when present) in the pyridine substituted —OR, —SR, —NR—, —NHR or —NR₂ is independently a linear or branched C₁-C₅ alkyl. In some embodiments, R is methyl. In some embodiments, R is ethyl. In some embodiments, R is propyl. In some embodiments, R is isopropyl. In some embodiments, R is n-butyl. The trifluoroborate-containing prosthetic group(s) may comprise ¹⁸F. In some embodiments, one fluorine is a BF₃—R⁵— is ¹⁸F. In some embodiments, all three fluorines in a BF₃—R⁵— are ¹⁸F. In some embodiments, all three fluorines in a BF₃—R⁵— are ¹⁹F.

In some embodiments, a BF₃—R⁵— may independently form

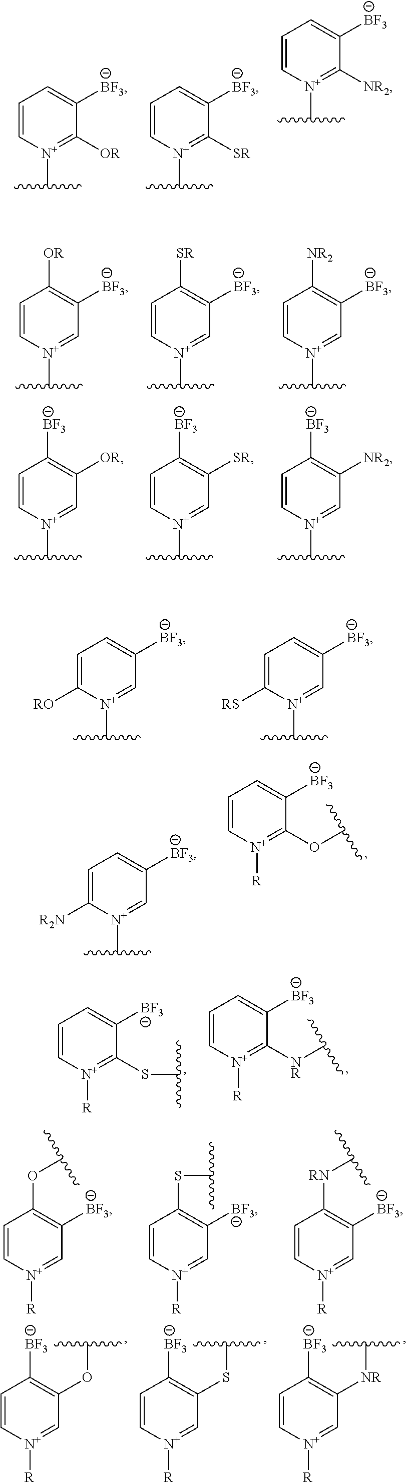

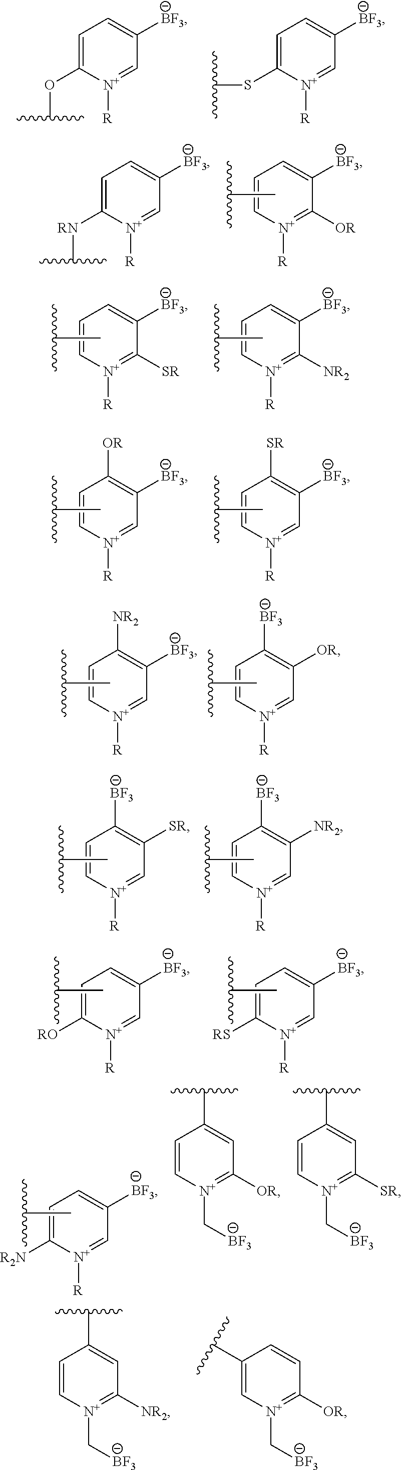

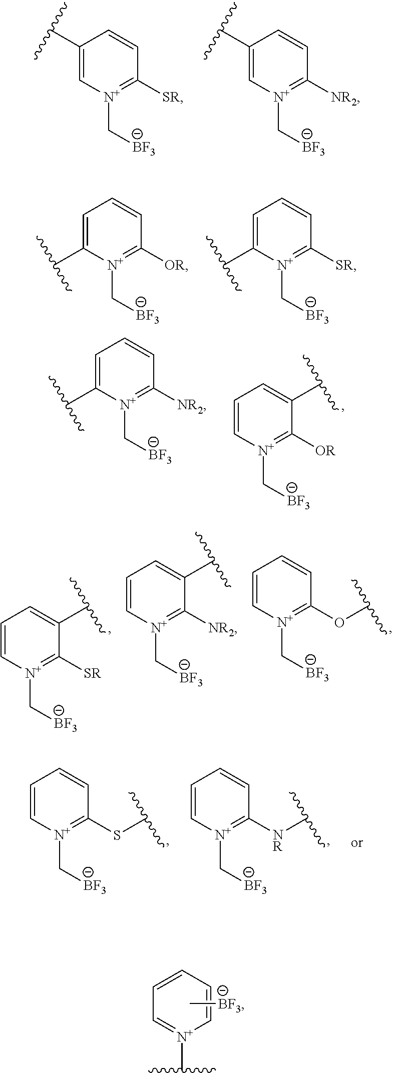

in which each R (when present) in the pyridine substituted —OR, —SR, —NR—, —NHR or —NR₂ is independently a linear or branched C₁-C₅ alkyl. In some embodiments, R is methyl. In some embodiments, R is ethyl. In some embodiments, R is propyl. In some embodiments, R is isopropyl. In some embodiments, R is n-butyl. In some embodiments, a BF₃—R⁵— is

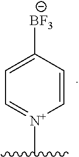

In some embodiments, all three fluorines in a BF₃—R⁵— are ¹⁸F. In some embodiments, one fluorine in a BF₃—R⁵— is ¹⁸F. In some embodiments, all three fluorines in a BF₃—R⁵— are ¹⁹F.

In some embodiments, at least one BF₃—R⁵— or optionally each BF₃—R⁵— is independently

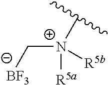

wherein R^{5a} and R^{5b} are each independently a C₁-C₅ linear or branched alkyl group. In some embodiments, R^{5a} is methyl. In some embodiments, R^{5a} is ethyl. In some embodiments, R^{5a} is propyl. In some embodiments, R^{5a} is isopropyl. In some embodiments, R^{5a} is butyl. In some embodiments, R^{5a} is n-butyl. In some embodiments, R^{5a} is pentyl. In some embodiments, R^{5b} is methyl. In some embodiments, R^{5b} is ethyl. In some embodiments, R^{5b} is propyl. In some embodiments, R^{5b} is isopropyl. In some embodiments, R^{5b} is butyl. In some embodiments, R^{5b} is n-butyl. In some embodiments, R^{5b} is pentyl. In some embodiments, R^{5a} and R^{5b} are both methyl. The trifluoroborate-containing prosthetic group may comprise ¹⁸F. In some embodiments, one fluorine in BF₃—R⁵— is ¹⁸F. In some embodiments, all three fluorines in BF₃—R⁵— are ¹⁸F. In some embodiments, all three fluorines in BF₃—R⁵— are ¹⁹F.

In certain embodiments, the compound is conjugated with a radionuclide for positron emission tomography (PET) or single photon emission computed tomography (SPECT) imaging of GRPR expressing tumors, wherein the compound is conjugated with a radionuclide that is a positron emitter or a gamma emitter. Without limitation, the positron or gamma emitting radionuclide is ⁶⁸Ga, ⁶⁷Ga, ⁶¹Cu, ⁶⁴Cu, ⁶⁷Ga, ^{99m}Tc, ^{110m}In, ¹¹¹In, ⁴⁴Sc, ⁸⁶Y, ⁸⁹Zr, ⁹⁰Nb, ¹⁵²Tb, ¹⁵⁵Tb, ¹⁸F, ¹³¹I, ¹²³I, ¹²⁴I, ²⁰³Pb and ⁷²As.

In certain embodiments the compound is conjugated with a radionuclide that is used for therapy. This includes radioisotopes such as ¹⁶⁵Er, ²¹²Bi, ²¹¹At, ¹⁶⁶Ho, ¹⁴⁹Pm, ¹⁵⁹Gd, ¹⁰⁵Rh, ¹⁰⁹Pd, ¹⁹⁸Au, ¹⁹⁹Au, ¹⁷⁵Yb, ¹⁴²Pr, ¹⁷⁷Lu, ¹¹¹In, ²¹³Bi, ⁴⁷Sc, ⁹⁰Y, ²²⁵Ac, ^{117m}Sn, ¹⁵³Sm, ¹⁴⁹Tb, ¹⁶¹Tb, ¹⁶⁵Er, ²¹³Bi, ²²⁴Ra, ²²³Ra, ²¹²Bi, ²¹²Pb, ²²⁵Ac, ²²⁷Th, ²²³Ra, ⁴⁷Sc, ⁷⁷As, ¹⁸⁶Re, ¹⁸⁸Re, ⁶⁴Cu or ⁶⁷Cu.

In some embodiments, n6 is 1, and the linker and R^{L} together form a p-aminomethylaniline-diglycolic acid (pABzA-DIG) linker, a 4-amino-(1-carboxymethyl)piperidine (Pip) linker, a 9-amino-4,7-dioxanonanoic acid (dPEG2) linker, or a 4-(2-aminoethyl)-1-carboxymethyl-piperazine (Acp) linker. In some embodiments, the linker and R^{L} together form:

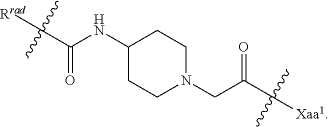

In some embodiments, the compound is LW01025, optionally conjugated by a radiometal. In some embodiments, the compound is LW01029, optionally conjugated by a radiometal. In some embodiments, the compound is LW01107, optionally conjugated by a radiometal. In some embodiments, the compound is LW01108, optionally conjugated by a radiometal. In some embodiments, the compound is LW01110, optionally conjugated by a radiometal. In some embodiments, the compound is LW01102, optionally conjugated by a radiometal. In some embodiments, the compound is LW01142, optionally conjugated by a radiometal. In some embodiments, the compound is LW01158, optionally conjugated by a radiometal. In some embodiments, the compound is LW01186, optionally conjugated by a radiometal.

In some embodiments, the compound is LW02002, optionally conjugated by a radiometal. In some embodiments, the compound is LW02021, optionally conjugated by a radiometal. In some embodiments, the compound is LW02023, optionally conjugated by a radiometal. In some embodiments, the compound is LW02025, optionally conjugated by a radiometal.

In some embodiments, the compound is LW01045, optionally conjugated by a radiometal. In some embodiments, the compound is LW01059, optionally conjugated by a radiometal. In some embodiments, the compound is LW01061, optionally conjugated by a radiometal. In some embodiments, the compound is LW01090, optionally conjugated by a radiometal. In some embodiments, the compound is LW01117, optionally conjugated by a radiometal.

In some embodiments, the compound is:
LW01025 (DOTA-Pip-D-Phe-Gln-Trp-Ala-Val-Gly-His-Leu-Thz-NH₂);
LW01029 (DOTA-Pip-D-2-Nal-Gln-Trp-Ala-Val-Gly-His-Leu-Thz-NH₂);
LW01107 (DOTA-Pip-D-Phe-Gln-Trp-Ala-Val-Gly-NMe-His-Leu-Thz-NH₂);
LW01108 (DOTA-Pip-D-Phe-Gln-Trp-Ala-Tle-Gly-His-Leu-Thz-NH₂);
LW01110 (DOTA-Pip-D-Phe-Gln-Trp-Ala-Tle-Gly-NMe-His-Leu-Thz-NH₂);
LW01142 (DOTA-Pip-D-Phe-His-Trp-Ala-Tle-Gly-NMe-His-Leu-Thz-NH₂);
LW01102 (DOTA-Pip-D-Phe-His-Trp-Ala-Val-Gly-His-LeuψThz-NH₂);
LW01158 (DOTA-Pip-D-Phe-Gln-Trp-Ala-Tle-Gly-His-LeuψThz-NH₂);
LW01080 (D-Phe-Gln-Trp-Ala-Tle-Gly-His-Leu-Thz-NH₂);
LW01085 (D-Phe-Gln-Trp-Ala-Val-Gly-His-Leu-Thz-NH₂);
LW01088 (D-Phe-Gln-Trp-Ala-Val-Gly-NMe-His-Leu-Thz-NH₂);
LW01136 (D-Phe-Gln-Trp(Me)-Ala-Val-Gly-His-Leu-Thz-NH₂);
LW01186 (DOTA-Pip-D-Phe-Gln-αMe-Trp-Ala-Tle-Gly-His-LeuψThz-NH₂);
LW02002 (DOTA-Pip-D-Phe-Gln-Trp-Ala-Tle-N-Me-Gly-His-LeuψThz-NH₂);
LW02021 (DOTA-Pip-D-Phe-Gln-7-F-Trp-Ala-Val-Gly-His-Leu-Thz-NH₂);
LW01142 (DOTA-Pip-D-Phe-His-Trp-Ala-Tle-Gly-NMe-His-Leu-Thz-NH₂);
LW02023 (DOTA-Pip-D-Phe-Gln-5-Me-Trp-Ala-Val-Gly-His-Leu-Thz-NH₂);
LW02025 (DOTA-Pip-D-Phe-Gln-2-Me-Trp-Ala-Val-Gly-His-Leu-Thz-NH₂);
LW02045 (DOTA-Pip-D-Phe-Gln-Trp-Ala-Val-N-MeGly-His-LeuψPro-NH₂);
LW02042 (DOTA-Pip-D-Phe-Gln-Trp-Ala-Val-Gly-His-Phe-Thz-NH₂);
LW02011 (D-Phe-Gln-Trp-Ala-2,3-dehydro-Val-Gly-His-Leu-Thz-NH₂);
LW02016 (D-Phe-Gln-Trp-Ala-L-cyclopropylycine-Gly-His-Leu-Thz-NH₂);
LW02019 (D-Phe-Gln-Trp-Ala-cyclobutaneacetic acid-Gly-His-Leu-Thz-NH₂);
LW01166 (D-Phe-Gln-5-F-Trp-Ala-Val-Gly-His-Leu-Thz-NH₂);
LW01171 (D-Phe-Gln-6-Me-Trp-Ala-Val-Gly-His-Leu-Thz-NH₂);
LW01173 (D-Phe-Gln-5-OH-Trp-Ala-Val-Gly-His-Leu-Thz-NH₂);
LW01175 (D-Phe-Gln-6-F-Trp-Ala-Val-Gly-His-Leu-Thz-NH₂);
LW01177 (D-Phe-Gln-7-F-Trp-Ala-Val-Gly-His-Leu-Thz-NH₂);
LW01180 (D-Phe-Gln-4-F-Trp-Ala-Val-Gly-His-Leu-Thz-NH₂);
LW01182 (D-Phe-Gln-5-Me-Trp-Ala-Val-Gly-His-Leu-Thz-NH₂);
LW01183 (D-Phe-Gln-4-Me-Trp-Ala-Val-Gly-His-Leu-Thz-NH₂);
LW01191 (D-Phe-Gln-D-Tpi-Ala-Val-Gly-His-Leu-Thz-NH₂);
LW02007 (D-Phe-Gln-7-Me-Trp-Ala-Val-Gly-His-Leu-Thz-NH₂);
LW02009 (D-Phe-Gln-2-Me-Trp-Ala-Val-Gly-His-Leu-Thz-NH₂);
LW02013 (D-Phe-Gln-7-Aza-Trp-Ala-Val-Gly-His-Leu-Thz-NH₂); or
LW02015 (D-Phe-Gln-Bta-Ala-Val-Gly-His-Leu-Thz-NH₂), wherein y for these compounds is a reduced peptide bond.

In another embodiment, the compound is:
LW01080* (DOTA-Pip-D-Phe-Gln-Trp-Ala-Tle-Gly-His-Leu-Thz-NH₂);
LW01085* (DOTA-Pip-D-Phe-Gln-Trp-Ala-Val-Gly-His-Leu-Thz-NH₂);
LW01088* (DOTA-Pip-D-Phe-Gln-Trp-Ala-Val-Gly-NMe-His-Leu-Thz-NH₂);
LW01136* (DOTA-Pip-D-Phe-Gln-Trp(Me)-Ala-Val-Gly-His-Leu-Thz-NH₂);
LW02011* (DOTA-Pip-D-Phe-Gln-Trp-Ala-2,3-dehydro-Val-Gly-His-Leu-Thz-NH₂);
LW02016* (DOTA-Pip-D-Phe-Gln-Trp-Ala-L-cyclopropylycine-Gly-His-Leu-Thz-NH₂);
LW02019* (DOTA-Pip-D-Phe-Gln-Trp-Ala-cyclobutaneacetic acid-Gly-His-Leu-Thz-NH₂);
LW01166* (DOTA-Pip-D-Phe-Gln-5-F-Trp-Ala-Val-Gly-His-Leu-Thz-NH₂);
LW01171* (DOTA-Pip-D-Phe-Gln-6-Me-Trp-Ala-Val-Gly-His-Leu-Thz-NH₂);
LW01173* (DOTA-Pip-D-Phe-Gln-5-OH-Trp-Ala-Val-Gly-His-Leu-Thz-NH₂);
LW01175* (DOTA-Pip-D-Phe-Gln-6-F-Trp-Ala-Val-Gly-His-Leu-Thz-NH₂);
LW01177* (DOTA-Pip-D-Phe-Gln-7-F-Trp-Ala-Val-Gly-His-Leu-Thz-NH₂);
LW01180* (DOTA-Pip-D-Phe-Gln-4-F-Trp-Ala-Val-Gly-His-Leu-Thz-NH₂);
LW01182* (DOTA-Pip-D-Phe-Gln-5-Me-Trp-Ala-Val-Gly-His-Leu-Thz-NH₂);
LW01183 (DOTA-Pip-D-Phe-Gln-4-Me-Trp-Ala-Val-Gly-His-Leu-Thz-NH₂);
LW01191* (DOTA-Pip-D-Phe-Gln-D-Tpi-Ala-Val-Gly-His-Leu-Thz-NH₂);
LW02007* (DOTA-Pip-D-Phe-Gln-7-Me-Trp-Ala-Val-Gly-His-Leu-Thz-NH₂);
LW02009* (DOTA-Pip-D-Phe-Gln-2-Me-Trp-Ala-Val-Gly-His-Leu-Thz-NH₂);
LW02013* (DOTA-Pip-D-Phe-Gln-7-Aza-Trp-Ala-Val-Gly-His-Leu-Thz-NH₂); or
LW02015* (DOTA-Pip-D-Phe-Gln-Bta-Ala-Val-Gly-His-Leu-Thz-NH₂), wherein Ly for these compounds is a reduced peptide bond.

In another embodiment, the compound is:
LW01045 (DOTA-Pip-D-Phe-Gln-Trp-Ala-Val-Gly-His-Leu-ψ-Thz-NH₂),
LW01059 (DOTA-Pip-D-2-Nal-Gln-Trp-Ala-Val-Gly-His-Leu-ψ-Thz-NH₂),
LW01061 (DOTA-Pip-D-Tpi-Gln-Trp-Ala-Val-Gly-His-Leu-ψ-Thz-NH₂),
LW01090 (DOTA-Pip-D-Phe-Gln-Trp-Ala-Val-NMe-Gly-His-Leu-ψ-Thz-NH₂), or
LW01117 (DOTA-Cysteic acid-Pip-D-2-NaI-Gln-Trp-Ala-Val-Gly-His-Leu-ψ-Thz-NH₂), wherein y for these compounds is a reduced peptide bond.

In another specific embodiment, the LW0 compounds listed above and described herein may be included in a pharmaceutical composition. In a specific embodiment, the pharmaceutical composition may include one or more compounds from LW0 compounds listed above and described herein or Formula I, A, or B and a pharmaceutically acceptable carrier. In another specific embodiment, the compound(s) may be bound to or include a radiometal. In a specific embodiment, the radiometal is ¹⁶⁵Er, ²¹²Bi, ²¹¹At, ¹⁶⁶Ho, ¹⁴⁹Pm, ¹⁵⁹Gd, ¹⁰⁵Rh, ¹⁰⁹Pd, ¹⁹⁸Au, ¹⁹⁹Au, ¹⁷⁵Yb, ¹⁴²Pr, ¹⁷⁷Lu, ¹¹¹In, ²¹³Bi, ⁴⁷Sc, ⁹⁰Y, ²²⁵Ac, ^{117m}Sn, ¹⁵³Sm, ¹⁴⁹Tb, ¹⁶¹Tb, ²²⁴Ra, ²²³Ra, ²¹²Pb, ²²⁷Th, ²²³Ra, ⁷⁷As, ¹⁸⁶Re, ¹⁸⁸Re, ⁶⁷Cu, or ⁶⁴Cu.

In a specific embodiment, the LW0 compounds listed above and described herein, may used for imaging methods. In a specific embodiment, the methods may include imaging Gastrin-releasing peptide receptor (GRPR) in a subject, the method comprising: administering to the subject a peptidic compound, including the LW0 compounds listed above and described herein, and/or any compound of Formulas I, A or B; and imaging tissue of the subject. In a specific embodiment, the methods may include the methods of treating cancer in a subject comprising, administering to the subject in need thereof a peptidic compound including the LW0 compounds listed above and described herein, and/or any compound of Formulas I, A or B.

In another specific embodiment, the methods may include treating a GRPR-expressing condition or disease. In a specific embodiment, the GRPR-expressing condition or disease may be a psychiatric disorder, neurological disorder, inflammatory disease, prostate cancer, lung cancer, head and neck cancer, colon cancer, kidney cancer, ovarian cancer, liver cancer, pancreatic cancer, breast cancer, glioma or neuroblastoma. In some embodiments, the cancer is prostate cancer.

In some embodiments, the compounds described herein are optionally conjugated by a radiometal, and may be used for the methods described herein.

In alternative embodiments, the radiometal is ¹⁷⁷Lu, ¹¹¹In, ²¹³Bi, ⁶⁸Ga, ⁶⁷Ga, ²⁰³Pb, ²¹²Pb, ⁴⁴Sc, ⁴⁷Sc, ⁹⁰Y, ⁸⁶Y, ²²⁵Ac, ^{117m}Sn, ¹⁵³Sm, ¹⁴⁹Tb, ¹⁵²Tb, ¹⁵⁵Tb, ¹⁶¹Tb, ¹⁶⁵Er, ²¹³Bi, ²²⁴Ra, ²¹²Bi, ²¹²Pb, ²²⁵Ac, ²²⁷Th, ²²³Ra, ⁴⁷Sc, ⁶⁴Cu or ⁶⁷Cu. In some embodiments, the radiometal is ⁶⁸Ga. In some embodiments, the radiometal is ⁶⁴Cu. In some embodiments, the radiometal is ⁶⁷Cu. In some embodiments, the radiometal is ⁶⁷Ga. In some embodiments, the radiometal is ¹¹¹In. In some embodiments, the radiometal is ¹⁷⁷Lu. In some embodiments, the radiometal is ⁹⁰Y In some embodiments, the radiometal is ²²⁵Ac.

When the radiolabeling group (i.e. R^{rad} in Formula I) comprises or is conjugated to a diagnostic radionuclide, there is disclosed use of certain embodiments of a compound as disclosed herein for preparation of a radiolabelled tracer for imaging GRPR-expressing tissues in a subject. There is also disclosed a method of imaging GRPR-expressing tissues in a subject, in which the method comprises: administering to the subject a composition comprising a compound described herein and a pharmaceutically acceptable excipient; and imaging tissue of the subject, e.g. using PET or SPECT. When the tissue is a diseased tissue (e.g. a GRPR-expressing cancer), GRPR-targeted treatment may then be selected for treating the subject.

When the radiolabeling group (i.e. R^{rad} in Formula I) comprises or is conjugated to a therapeutic radionuclide, there is disclosed use of certain embodiments of the compound (or a pharmaceutical composition thereof) for the treatment of GRPR-expressing conditions or diseases (e.g. cancer and the like) in a subject. Accordingly, there is provided use of a compound disclosed herein in preparation of a medicament for treating a GRPR-expressing condition or disease in a subject. There is also provided a method of treating GRPR-expressing disease in a subject, in which the method comprises: administering to the subject a composition comprising the compound and a pharmaceutically acceptable excipient. For example, but without limitation, the disease may be a GRPR-expressing cancer. In a specific embodiment, the LW0 compounds listed above and described herein, may include or be conjugated to a radiometal. In a specific embodiment, the methods may include imaging Gastrin-releasing peptide receptor (GRPR) in a subject, the method comprising: administering to the subject a peptidic compound, including the LW0 compounds listed above with a radiometal and described herein, and/or any compound of Formulas I, A or B with a radiometal; and imaging tissue of the subject. In a specific embodiment, the methods may include the methods of treating cancer in a subject comprising, administering to the subject in need thereof a peptidic compound including the LW0 compounds listed above with a radiometal and described herein, and/or any compound of Formulas I, A or B with a radiometal.

Aberrant or ectopic GRPR expression has been detected in various conditions and diseases, including psychiatric/neurological disorders, inflammatory disease, and cancer (Cornelio, et al. Ann Oncol. 2007, 18:1457-1466; Bajo et al. Proc Natl Acad Sci USA. 2002, 99:3836-3841; Koppán et al. Cancer. 1998, 83:1335-1343; Shirahige et al. Biomed Pharmacother. 1994 48:465-472; Cai et al. Int J Oncol. 1995, 6:1165-1172; Jungwirth, Eur J Cancer Part A. 1997, 33:1141-1148; Gonzalez et al., J Pharmacol Exp Ther. 200, 331(1): 265-276; Dalm et al. PLoS One. 2017, 12(1): e0170536; Guo et al., Curr Opin Endocrinol Diabetes Obes. 2015, 22(1): 3-8; Ischia et al., BJU Int. 201,113 Suppl 2:40-47; Ramos-Alvarez et al. Peptide 2015, 72: 128-144). Accordingly, without limitation, the GRPR-expressing condition or disease may be psychiatric disorder, neurological disorder, inflammatory disease, prostate cancer, lung cancer, head and neck cancer, colon cancer, kidney cancer, ovarian cancer, liver cancer, pancreatic cancer, breast cancer, glioma or neuroblastoma. In some embodiments, the cancer is prostate cancer.

The compounds presented herein incorporate peptides, which may be synthesized by any of a variety of methods established in the art. This includes but is not limited to liquid-phase as well as solid-phase peptide synthesis using methods employing 9-fluorenylmethoxycarbonyl (Fmoc) and/or t-butyloxycarbonyl (Boc) chemistries, and/or other synthetic approaches.

Solid-phase peptide synthesis methods and technology are well-established in the art. For example, peptides may be synthesized by sequential incorporation of the amino acid residues of interest one at a time. In such methods, peptide synthesis is typically initiated by attaching the C-terminal amino acid of the peptide of interest to a suitable resin. Prior to this, reactive side chain and alpha amino groups of the amino acids are protected from reaction by suitable protecting groups, allowing only the alpha carboxyl group to react with a functional group such as an amine group, a hydroxyl group, or an alkyl halide group on the solid support. Following coupling of the C-terminal amino acid to the support, the protecting group on the side chain and/or the alpha amino group of the amino acid is selectively removed, allowing the coupling of the next amino acid of interest. This process is repeated until the desired peptide is fully synthesized, at which point the peptide can be cleaved from the support and purified. A non-limiting example of an instrument for solid-phase peptide synthesis is the Aapptec Endeavor 90 peptide synthesizer.

To allow coupling of additional amino acids, Fmoc protecting groups may be removed from the amino acid on the solid support, e.g. under mild basic conditions, such as piperidine (20-50% v/v) in DMF. The amino acid to be added must also have been activated for coupling (e.g. at the alpha carboxylate). Non-limiting examples of activating reagents include without limitation 2-(1H-benzotriazol-1-yl)-1,1,3,3-tetramethyluronium hexafluorophosphate (HBTU), 2-(1H-benzotriazol-1-yl)-1,1,3,3-tetramethyluronium tetrafluoroborate (TBTU), 2-(7-Aza-1H-benzotriazole-1-yl)-1,1,3,3-tetramethyluronium hexafluorophosphate (HATU), benzotriazole-1-yl-oxy-tris(dimethylamino)phosphoniumhexafluorophosphate (BOP), benzotriazole-1-yl-oxy-tris(pyrrolidino)phosphoniumhexafluorophosphate (PyBOP). Racemization is minimized by using triazoles, such as 1-hydroxy-benzotriazole (HOBt) and 1-hydroxy-7-aza-benzotriazole (HOAt). Coupling may be performed in the presence of a suitable base, such as N,N-diisopropylethylamine (DIPEA/DIEA) and the like.

Apart from forming typical peptide bonds to elongate a peptide, peptides may be elongated in a branched fashion by attaching to side chain functional groups (e.g. carboxylic acid groups or amino groups), either: side chain to side chain; or side chain to backbone amino or carboxylate. Coupling to amino acid side chains may be performed by any known method, and may be performed on-resin or off-resin. Non-limiting examples include: forming an amide between an amino acid side chain containing a carboxyl group (e.g. Asp, D-Asp, Glu, D-Glu, and the like) and an amino acid side chain containing an amino group (e.g. Lys, D-Lys, Orn, D-Orn, Dab, D-Dab, Dap, D-Dap, and the like) or the peptide N-terminus; forming an amide between an amino acid side chain containing an amino group (e.g. Lys, D-Lys, Orn, D-Orn, Dab, D-Dab, Dap, D-Dap, and the like) and either an amino acid side chain containing a carboxyl group (e.g. Asp, D-Asp, Glu, D-Glu, and the like) or the peptide C-terminus; and forming a 1, 2, 3-triazole via click chemistry between an amino acid side chain containing an azide group (e.g. Lys(N₃), D-Lys(N₃), and the like) and an alkyne group (e.g. Pra, D-Pra, and the like). The protecting groups on the appropriate functional groups must be selectively removed before amide bond formation, whereas the reaction between an alkyne and an azido groups via the click reaction to form an 1,2,3-triazole does not require selective deprotection. Non-limiting examples of selectively removable protecting groups include 2-phenylisopropyl esters (O-2-PhiPr) (e.g. on Asp/Glu) as well as 4-methyltrityl (Mtt), allyloxycarbonyl (alloc), 1-(4,4-dimethyl-2,6-dioxocyclohex-1-ylidene))ethyl (Dde), and 1-(4,4-dimethyl-2,6-dioxocyclohex-1-ylidene)-3-methylbutyl (ivDde) (e.g. on Lys/Orn/Dab/Dap). O-2-PhiPr and Mtt protecting groups can be selectively deprotected under mild acidic conditions, such as 2.5% trifluoroacetic acid (TFA) in DCM. Alloc protecting groups can be selectively deprotected using tetrakis(triphenylphosphine)palladium(0) and phenyl silane in DCM. Dde and ivDde protecting groups can be selectively deprotected using 2-5% of hydrazine in DMF. Deprotected side chains of Asp/Glu (L- or D-forms) and Lys/Orn/Dab/Dap (L- or D-forms) can then be coupled, e.g. by using the coupling reaction conditions described above. The above provides means for including multiple BF₃ groups.

Peptide backbone amides may be N-methylated (i.e. alpha amino methylated) or N-alkylated. This may be achieved by directly using Fmoc-N-methylated (or Fmoc-N-alkylated) amino acids during peptide synthesis. Alternatively, N-methylation under Mitsunobu conditions may be performed. First, a free primary amine group is protected using a solution of 4-nitrobenzenesulfonyl chloride (Ns-Cl) and 2,4,6-trimethylpyridine (collidine) in NMP. N-methylation (or N-alkylation) may then be achieved in the presence of triphenylphosphine, diisopropyl azodicarboxylate (DIAD) and methanol. Subsequently, N-deprotection may be performed using mercaptoethanol and 1,8-diazabicyclo[5.4.0]undec-7-ene (DBU) in NMP. For coupling protected amino acids to N-methylated (or N-alkylated) alpha amino groups, HATU, HOAt and DIEA may be used.

The formation of the thioether (—S—) linkages (e.g. for L¹) can be achieved either on solid phase or in solution phase. For example, the formation of thioether (—S—) linkage can be achieved by coupling between a thiol-containing compound (such as the thiol group on cysteine side chain) and an alkyl halide (such as 3-(Fmoc-amino)propyl bromide and the like) in an appropriate solvent (such as N,N-dimethylformamide and the like) in the presence of base (such as N,N-diisopropylethylamine and the like). If the reactions are carried out in solution phase, the reactants used are preferably in equivalent molar ratio (1 to 1), and the desired products can be purified by flash column chromatography or high performance liquid chromatography (HPLC). If the reactions are carried out on solid phase, meaning one reactant has been attached to a solid phase, then the other reactant is normally used in excess amount (3 equivalents of the reactant attached to the solid phase). After the reactions, the excess unreacted reactant and reagents can be removed by sequentially washing the solid phase (resin) using a combination of solvents, such as N,N-dimethylformamide, methanol and dichloromethane, for example.

The formation of the linkage (e.g. for L¹) between a thiol group and a maleimide group can be performed using the conditions described above for the formation of the thioether (—S—) linkage simply by replacing the alkyl halide with a maleimide-containing compounds. Similarly, this reaction can be conducted in solid phase or solution phase. If the reactions are carried out in solution phase, the reactants used are preferably in equivalent molar ratio (1 to 1), and the desired products can be purified by flash column chromatography or high performance liquid chromatography (HPLC). If the reactions are carried out on solid phase, meaning one reactant has been attached to a solid phase, then the other reactant is normally used in excess amount (3 equivalents of the reactant attached to the solid phase). After the reactions, the excess unreacted reactant and reagents can be removed by sequentially washing the solid phase (resin) using a combination of solvents, such as N,N-dimethylformamide, methanol and dichloromethane, for example.

Urea or thiourea linkages can be made from reaction of an amine group with an isocyanate or an isothiocyanate, respectively, which are common functional groups on radiometal chelators. The isothiocyanate functional group may be added to the radiometal chelator by reacting an amino group on the chelator with thiophosgene [i.e. C(S)Cl₂]. Similarly, the isocyanate functional group may be added to the radiometal chelator by reacting an amino group on the chelator with phosgene [i.e. C(O)Cl₂].

Non-peptide moieties (e.g. radiolabeling groups and/or albumin binders) may be coupled to the peptide N-terminus while the peptide is attached to the solid support. This is facile when the non-peptide moiety comprises an activated carboxylate (and protected groups if necessary) so that coupling can be performed on resin. For example, but without limitation, a bifunctional chelator, such as 1,4,7,10-tetraazacyclododecane-1,4,7,10-tetraacetic acid (DOTA) tris(tert-butyl ester) may be activated in the presence of N-hydroxysuccinimide (NHS) and N,N′-dicyclohexylcarbodiimide (DCC) for coupling to a peptide. Alternatively, a non-peptide moiety may be incorporated into the compound via a copper-catalyzed click reaction under either liquid or solid phase conditions. Copper-catalyzed click reactions are well established in the art. For example, 2-azidoacetic acid is first activated by NHS and DCC and coupled to a peptide. Then, an alkyne-containing non-peptide moiety may be clicked to the azide-containing peptide in the presence of Cu²⁺ and sodium ascorbate in water and organic solvent, such as acetonitrile (ACN) and DMF and the like. Non-peptide moieties may also be added in solution phase, which is routinely performed.

The synthesis of radiometal chelators is well-known and many chelators are commercially available (e.g. from Sigma-Aldrich™/Milipore Sigma™ and others). Protocols for conjugation of radiometals to the chelators is also well known (e.g. see Examples, below).

The synthesis of the BF₃—R⁵—R⁴— component of the compounds can be achieved following previously reported procedures (Liu et al. Angew Chem Int Ed 2014 53:11876-11880; Liu et al. J Nucl Med 2015 55:1499-1505; Liu et al. Nat Protoc 2015 10:1423-1432; Kuo et al. J Nucl Med, 2019 60:1160-1166; each of which is incorporated by reference in its entirety). Generally, the BF₃-containing motif can be coupled to the linker via click chemistry by forming a 1,2,3-triazole ring between a BF₃-containing azido (or alkynyl) group and an alkynyl (or azido) group on the linker, or by forming an amide linkage between a BF₃-containing carboxylate and an amino group on the linker. To make the BF₃-containing azide, alkyne or carboxylate, a boronic acid ester-containing azide, alkyne or carboxylate is first prepared following by the conversion of the boronic acid ester to BF₃ in a mixture of HCl, DMF and KHF₂. For alkyl BF₃, the boronic acid ester-containing azide, alkyne or carboxylate can be prepared by coupling boronic acid ester-containing alkyl halide (such as iodomethylboronic acid pinacol ester) with an amine-containing azide, alkyne or carboxylate (such as N,N-dimethylpropargylamine). For aryl BF₃, the boronic acid ester can be prepared via Suzuki coupling using aryl halide (iodine or bromide) and bis(pinacolato)diboron.

¹⁸F-Fluorination of the BF₃-containing compounds via ¹⁸F-¹⁹F isotope exchange reaction can be achieved following previously published procedures (Liu et al. Nat Protoc 2015 10:1423-1432, incorporated by reference in its entirety). Generally, ˜100 nmol of the BF₃-containing compound is dissolved in a mixture of 15 μl of pyridazine-HCl buffer (pH=2.0-2.5, 1 M), 15 μl of DMF and 1 μl of a 7.5 mM KHF₂ aqueous solution. ¹⁸F-Fluoride solution (in saline, 60 μl) is added to the reaction mixture, and the resulting solution is heated at 80° C. for 20 min. At the end of the reaction, the desired product can be purified by solid phase extraction or by reversed high performance liquid chromatography (HPLC) using a mixture of water and acetonitrile as the mobile phase.

When the peptide has been fully synthesized on the solid support, the desired peptide may be cleaved from the solid support using suitable reagents, such as TFA, tri-isopropylsilane (TIS) and water. Side chain protecting groups, such as Boc, pentamethyldihydrobenzofuran-5-sulfonyl (Pbf), trityl (Trt) and tert-butyl (tBu) are simultaneously removed (i.e. deprotection). The crude peptide may be precipitated and collected from the solution by adding cold ether followed by centrifugation. Purification and characterization of the peptides may be performed by standard separation techniques, such as high performance liquid chromatography (HPLC) based on the size, charge and polarity of the peptides. The identity of the purified peptides may be confirmed by mass spectrometry or other similar approaches. The inventions described herein are further represented by the following embodiments.

### Embodiments of the Present Invention

Embodiment 1. A peptidic compound, wherein the compound has the structure of Formula I or is a salt or solvate of Formula I,

R^{rad}ₙ₆-[linker]-R^{L}-Xaa¹-Xaa²-Xaa³-Xaa⁴-Xaa⁵-Xaa⁶-Xaa⁷-Xaa⁸-ψ-Xaa⁹-NH₂ (I)

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), or D-2-Nal;
Xaa² is Asn, Gln, homoserine (Hse), citrulline (Cit) or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(5-OH) or αMe-Trp;
Xaa⁴ is Ala or Ser;
Xaa⁵ is Val, Cpg (cyclopentylglycine) or tert-leucine (Tle);
Xaa⁶ is Gly, NMe-Gly, or D-Ala;
Xaa⁷ is His or NMe-His;
Xaa⁸ is Leu, D-Pro, or Phe;
Xaa⁹-NH₂ is a C-terminally amidated amino acid residue selected from Pro, Phe, oxazolidine-4-carboxylic acid (4-oxa-L-Pro), Me₂Thz (5,5-dimethyl-1,3-thiazolidine-4-carboxylic acid), or thiazoline-4-carboxylic acid (Thz);
ψ represents a peptide bond or reduced peptide bond joining Xaa⁸ to Xaa⁹;
excluding compounds in which Xaa², Xaa³, Xaa⁵, and Xaa⁷ are Gln, Trp, Val, and His, respectively, in which L is a reduced peptide bond;
R^{L} is —C(O)—, —NH—C(O)—, or —NH—C(S)—;
the linker is a linear or branched chain of n1 units of -L¹R¹— and/or -(L¹)₂R¹—, wherein:
   n1 is 1-20;
   each R¹ is, independently, a linear, branched, and/or cyclic Cₙ₂ alkylenyl, alkenylenyl and/or alkynylenyl, wherein each n2 is independently 1-20, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid;
   L¹ bonds to carbon, wherein each L¹ is independently —S—, —N(R²)C(O)—, —C(O)N(R²)—, —NH—C(O)—NH—, —NH—C(S)—NH—,

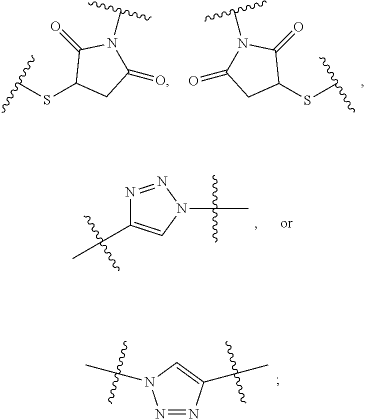

and
R² is H, methyl or ethyl; and
an albumin binder (R^{alb}) is optionally bonded to an L¹ of the linker, wherein the albumin binder is:
   —(CH₂)ₙ₃—CH₃ wherein n3 is 8-20;
   —(CH₂)ₙ₄—C(O)OH wherein n4 is 8-20;

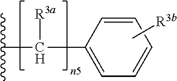

wherein n5 is 1-4 and R^{3a} is H or methyl, and R^{3b} is I, Br, F, Cl, H, OH, OCH₃, NH₂, NO₂ or C₁-C₆ alkyl; or

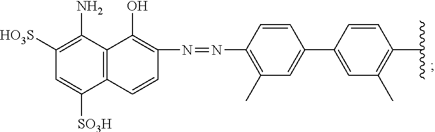 [SMILES: *c1ccc(-c2ccc(N=Nc3ccc4c(S(=O)(=O)O)cc(S(=O)(=O)O)c(N)c4c3O)c(C)c2)cc1C]

n6 is 1-5; and
each R^{rad} is a radiolabeling group bonded to or incorporating an L¹ of the linker, wherein each radiolabeling group is independently: a radiometal chelator; an aryl or heteroaryl substituted with a radiohalogen; a prosthetic group containing a trifluoroborate; a prosthetic group containing a silicon-fluorine-acceptor moiety; or a prosthetic group containing a fluorophosphate, fluorosulfate, sulfonyl fluoride, or a combination thereof.

Embodiment 2. The peptidic compound of Embodiment 1, wherein ψ is a peptide bond.

Embodiment 3. The peptidic compound of Embodiment 1 or 2, wherein Xaa³ is αMe-Trp.

Embodiment 4. The peptidic compound of any one of Embodiments 1 to 3, wherein Xaa⁵ is Tle.

Embodiment 5. The peptidic compound of any one of Embodiments 1 to 4, wherein Xaa⁷ is NMe-His.

Embodiment 6. The peptidic compound of any one of Embodiments 1 to 5, wherein Xaa¹ is D-Phe or D-2-Nal.

Embodiment 7. The peptidic compound of any one of Embodiments 1 to 6, wherein Xaa² is Gln or His.

Embodiment 8. The peptidic compound of any one of Embodiments 1 to 7, wherein Xaa³ is Trp.

Embodiment 9. The peptidic compound of any one of Embodiments 1 to 8, wherein Xaa⁶ is Gly.

Embodiment 10. The peptidic compound of any one of Embodiments 1 to 9, wherein Xaa⁸ is Leu.

Embodiment 11. The peptidic compound of any one of Embodiments 1 to 10, wherein Xaa⁹ is Thz.

Embodiment 12. The peptidic compound of any one of Embodiments 1 to 11, wherein at least one R^{rad} is a radiometal chelator, optionally selected from the group consisting of: DOTA and derivatives; DOTAGA; NOTA; NODAGA; NODASA; CB-DO2A; 3p-C-DEPA; TCMC; DO3A; DTPA and DTPA analogues optionally selected from CHX-A″-DTPA and 1B4M-DTPA; TETA; NOPO; Me-3,2-HOPO; CB-TE1A1P; CB-TE2P; MM-TE2A; DM-TE2A; sarcophagine and sarcophagine derivatives optionally selected from SarAr, SarAr-NCS, diamSar, AmBaSar, and BaBaSar; TRAP; AAZTA; DATA and DATA derivatives; H2-macropa or a derivative thereof; H₂dedpa, H₄octapa, H₄py4pa, H₄Pypa, H₂azapa, H₅decapa, and other picolinic acid derivatives; CP256; PCTA; C-NETA; C-NE3TA; HBED; SHBED; BCPA; CP256; YM103; desferrioxamine (DFO) and DFO derivatives; H₆phospa; a trithiol chelate; mercaptoacetyl; hydrazinonicotinamide; dimercaptosuccinic acid; 1,2-ethylenediylbis-L-cysteine diethyl ester; methylenediphosphonate; hexamethylpropyleneamineoxime; hexakis(methoxy isobutyl isonitrile), H4py4pa-phenyl-NCS, and Crown.

Embodiment 13. The peptidic compound of Embodiment 12, wherein the radiometal chelator is bound by a radiometal, a radionuclide-bound metal, or a radionuclide-bound metal-containing prosthetic group, optionally selected from the group consisting of: ⁶⁸Ga, ⁶¹Cu, ⁶⁴Cu, ⁶⁷Cu, ⁶⁷Ga, ¹¹¹In, ⁴⁴Sc, ⁸⁶Y, ⁸⁹Zr, ⁹⁰Nb, ¹⁷⁷Lu, ^{117m}Sn, ¹⁶⁵Er, ⁹⁰Y, ²²⁷Th, ²²⁵Ac, ²¹³Bi, ²¹²Bi, ⁷²As, ⁷⁷As, ²¹¹At, ²⁰³Pb, ²¹²Pb, ⁴⁷Sc, ¹⁶⁶Ho, ¹⁸⁸Re, ¹⁸⁶Re, ¹⁴⁹Pm, ¹⁵⁹Gd, ¹⁰⁵Rh, ¹⁰⁹Pd, ¹⁹⁸Au, ¹⁹⁹Au, ¹⁷⁵Yb, ¹⁴²Pr, ^{114m}In, ^{94m}Tc, ^{99m}Tc, ¹⁴⁹Tb, ¹⁵²Tb, ¹⁵⁵Tb, ¹⁶¹Tb, and [¹⁸F]AlF.

Embodiment 14. The peptidic compound of any one of Embodiments 1 to 13, wherein at least one R^{rad} is a trifluoroborate containing prosthetic group BF₃—R⁵—R⁴—, wherein R⁴ is —(CH₂)₁₋₅— and optionally methylene, and wherein BF₃—R⁵— forms:

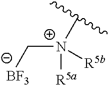

wherein R^{5a} and R^{5b} are each independently a C₁-C₅ linear or branched alkyl group,

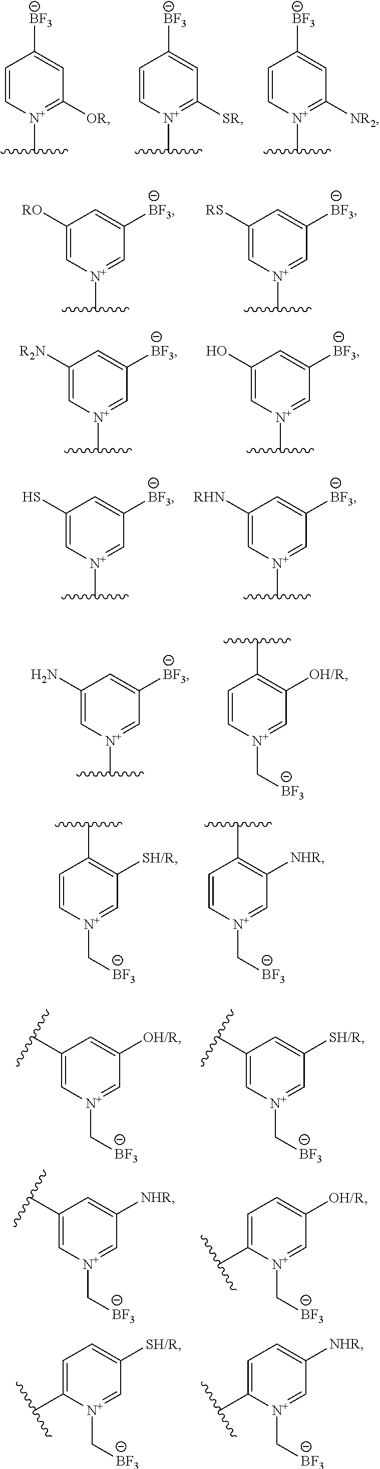

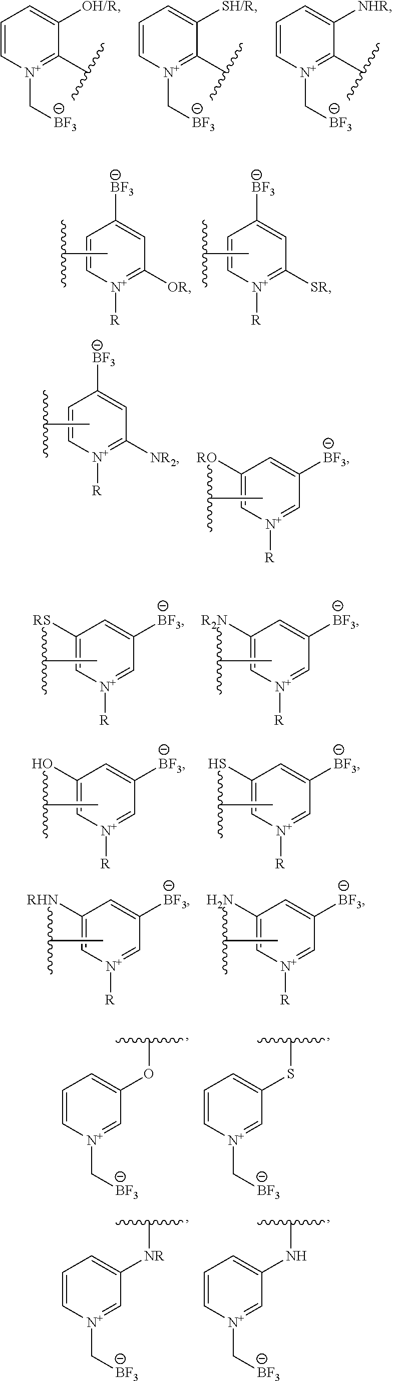

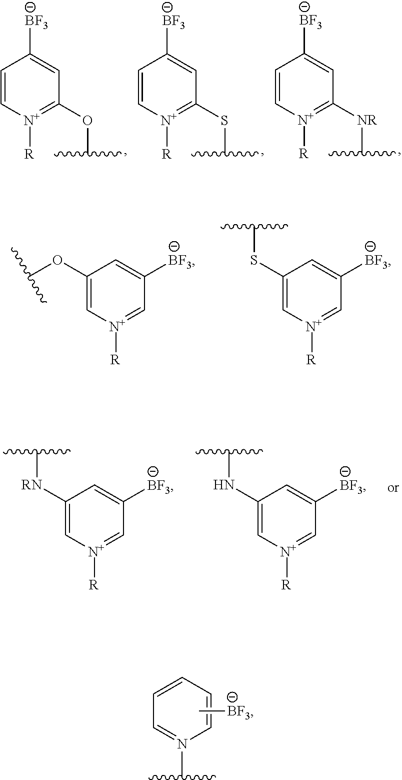

in which the R in each pyridine substituted —OR, —SR, —NR—, —NHR or —NR₂ is independently a branched or linear C₁-C₅ alkyl, optionally wherein the fluorines in BF₃—R⁵—R⁴— comprise ¹⁸F.

Embodiment 15. The peptidic compound of any one of Embodiment 1 to 11, wherein n6 is 2 and R^{rad}ₙ₆ comprises a first R^{rad} and a second R^{rad} wherein the first R^{rad} is a radiometal chelator as defined in Embodiment 12, optionally bound by a radiometal, a radionuclide-bound metal, or a radionuclide-bound metal-containing prosthetic group as defined in Embodiment 13, and wherein the second R^{rad} is a trifluoroborate containing prosthetic group as defined in Embodiment 14 or an aryl or heteroaryl substituted with a radiofluoride.

Embodiment 16. The peptidic compound of any one of Embodiments 1 to 15, wherein the linker and R^{L} together form a linear or branched peptide linker (Xaa¹⁰)₁₋₂₀, wherein each Xaa¹⁰ is independently a proteinogenic or non-proteinogenic amino acid residue, wherein each peptide backbone amino group is independently optionally methylated, and wherein each non-proteinogenic amino acid residue is independently selected from Table 1.

Embodiment 17. The peptidic compound of any one of Embodiments 1 to 11, wherein n6 is 1, and wherein the linker and R^{L} together form a p-aminomethylaniline-diglycolic acid (pABzA-DIG) linker, a 4-amino-(1-carboxymethyl)piperidine (Pip) linker, a 9-amino-4,7-dioxanonanoic acid (dPEG2) linker, or a 4-(2-aminoethyl)-1-carboxymethyl-piperazine (Acp) linker, optionally wherein the linker and R^{L} together form:

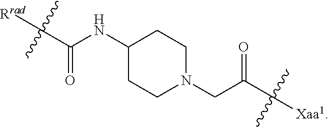

The present invention will be further illustrated in the following examples:

### Example 1

### General Methods

Chemicals were procured from commercial sources and used without further purification. All peptides were synthesized on an AAPPTec (Louisville, KY) Endeavor 90 peptide synthesizer. Purification and quality control of radiolabeling precursor, nonradioactive Ga-complexed standards and ⁶⁸Ga-labeled peptides were performed on Agilent (Santa Clara, CA) HPLC systems equipped with a model 1200 quaternary pump, a model 1200 UV absorbance detector (set at 220 nm), and a Bioscan (Washington, DC) Nal scintillation detector. The operation of Agilent HPLC systems was controlled using the Agilent ChemStation software. HPLC columns used were a semipreparative column (Luna C18, 5 μm particle size, 100 Å pore size, 250×10 mm) and an analytical column (Luna C18, 5 μm particle size, 100 Å pore size, 250×4.6 mm) from Phenomenex (Torrance, CA). The collected HPLC eluates containing the desired peptides were lyophilized using a Labconco (Kansas City, MO) FreeZone 4.5 Plus freeze drier. Mass analyses were performed using a Waters (Milford, MA) ACQUITY QDa mass spectrometer equipped with a 2489 UV/Vis detector, and an e2695 Separations module. C18 Sep-Pak cartridges (1 cm³, 50 mg) were obtained from Waters (Milford, MA). ⁶⁸Ga was eluted from an iThemba Laboratories (Somerset West, South Africa) generator and purified using a DGA resin column from Eichrom Technologies LLC (Lisle, IL). Radioactivity of ⁶⁸Ga-labeled peptides was measured using a Capintec (Ramsey, NJ) CRC-25R/W dose calibrator. PET/CT imaging was performed using a Siemens Inveon (Knoxville, TN) micro PET/CT scanner. The radioactivity of mouse tissues collected from biodistribution studies was counted using a PerkinElmer (Waltham, MA) Wizard2 2480 automatic gamma counter.

### Synthesis of Fmoc-LeuψThz-OH (4) Wherein ψ is a Reduced Peptide Bond

Compound 4 was synthesized following the reaction steps depicted in Scheme 1, shown below:

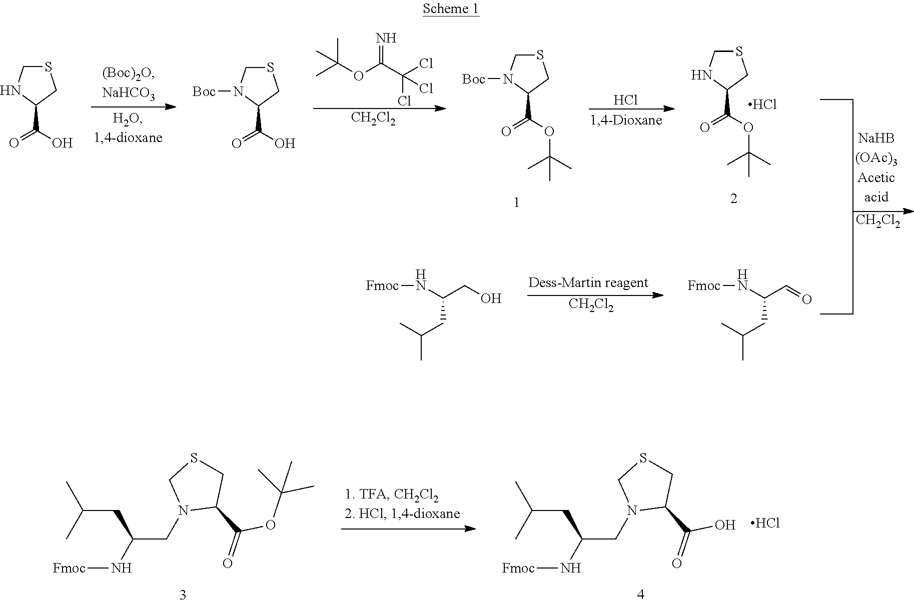

Synthesis of Boc-Thz-OtBu 1: Thiazolidine-4-carboxylic acid (2.66 g, 20 mmol), di-tert-butyl dicarbonate (4.37 g, 20 mmol), and sodium bicarbonate (2.52 g, 30 mmol) were stirring in water (40 mL) and 1,4-dioxane (40 mL) at room temperature overnight. The reaction mixture was washed with ether (100 mL×2) and the aqueous layer was collected. The collected aqueous fraction was adjusted to pH 3 by using concentrated hydrochloric acid before extracted with ethyl acetate (100 mL×2), dried over magnesium sulfate, filtered, and evaporated to obtain white solid. The obtained white solid was dissolved in 30 mL dichloromethane with tert-butyl 2,2,2-trichloroacetimidate (8.74 g, 40 mmol) and the mixture was stirred for 48 hours at room temperature. The mixture was filtered and the filtrate was concentrated in vacuo and purified by flash column chromatography eluted with 1:5 ethyl acetate/hexane to obtain compound 1 (4.36 g, 75% yield) as a colorless oil.

Synthesis of Thz-OtBu HCl salt (2): Compound 1 (4.31 g) was dissolved in a mixture of ethyl acetate (56.3 mL) and 4M HCl in 1,4-dioxane (18.8 mL), and stirred for 4 hours at room temperature. The precipitate was collected by filtration to obtain 2 as a white solid (1.78 g, 53% yield).

Synthesis of Fmoc-LeuψThz-OtBu (3) wherein ψ is a reduced peptide bond: Solution 1: Fmoc-Leucinol (3.79 g, 11.1 mmol) was converted to aldehyde by treating with Dess-Martin periodinane (5.87 g, 13.8 mol) in dichloromethane (70 mL) under ice/water bath for 4 hours. The reaction mixture was then mixed with saturated NaHCO₃ aqueous solution (130 mL) and sodium thiosulfate (13.0 g) and stirred for 30 min before being extracted with dichloromethane (130 mL). The organic layer was collected, dried over anhydrous magnesium sulfate, concentrated in vacuo to ˜20 mL in volume.

Solution 2: Compound 2 was dissolved in saturated NaHCO₃ aqueous solution (35 mL) and the mixture was extracted with ethyl acetate (100 mL×2). The organic phases were combined, dried over anhydrous magnesium sulfate, evaporated in vacuo to obtain colorless oil. The oil was mixed with acetic acid (400 μL, 7.0 mmol) in dichloromethane (30 mL).

Solutions 1 and 2 were mixed and the mixture was stirred for 30 min at room temperature. Sodium triacetoxyborohydride (5.41 g, 25.5 mmol) was added into the mixture and stirred for 20 hours. Saturated NaHCO₃ aqueous solution (100 mL) was added and stirred for 10 min. The mixture was extracted with ethyl acetate (100 mL×2). The organic phases were combined, dried over anhydrous MgSO₄ and purified by flash column chromatography eluted with 1:3 ether/hexane to obtain 3 as a white solid (2.13 g, 63% yield).

Synthesis of Fmoc-LeuψThz-OH (4) wherein y is a reduced peptide bond: Compound 3 was dissolved in a mixture of dichloromethane (25 mL) and trifluoroacetic acid (75 mL) and stirred for 3 hours at room temperature. After concentrated in vacuo, the residue was dissolved in ethyl acetate (80 mL) and mixed with 4M HCl in 1,4-dioxane dioxane (3 mL). After being stirred for 10 min, the volatile solvents were removed in vacuo. Diethyl ether (250 mL) was added to the residue and the mixture was stirred for 30 min. The formed white solid was collected by filtration to obtain 1.38 g of 4 (70% yield). ESI-MS: calculated [M+H]⁺ for 4 C₂₅H₃₀N₂O₄S 455.59; found 455.42.

### Synthesis of LW01025 and LW01029

The chemical structures of LW01025 and LW01029 are shown below:

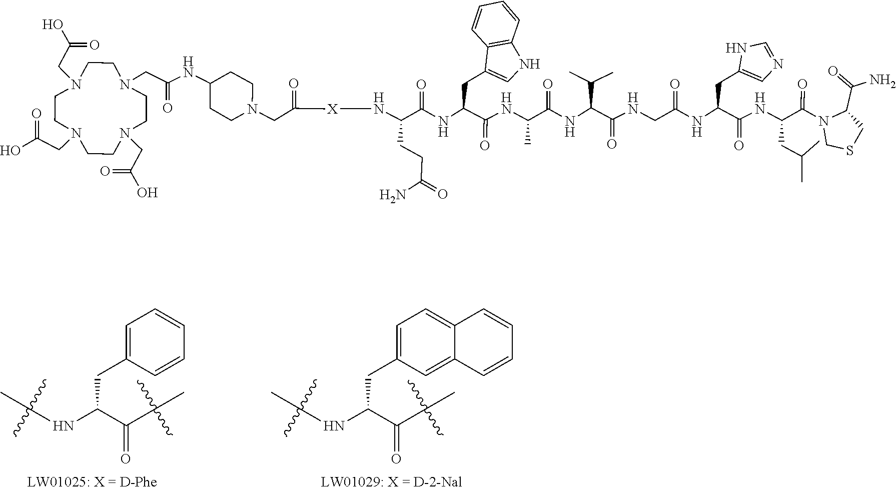

LW01025 (DOTA-Pip-D-Phe-Gln-Trp-Ala-Val-Gly-His-Leu-Thz-NH₂) and LW01029 (DOTA-Pip-D-2-Nal-Gln-Trp-Ala-Val-Gly-His-Leu-Thz-NH₂) were synthesized using standard Fmoc solid phase synthesis strategy starting from Fmoc-Rink Amide MBHA resin. Fmoc-Thz-OH (Fmoc-L-thiazolidine-4-carboxylic acid), Fmoc-Leu-OH, Fmoc-His(Trt)-OH, Fmoc-Gly-OH, Fmoc-Val-OH, Fmoc-Ala-OH, Fmoc-Trp(Boc)-OH, Fmoc-Gln(Trt)-OH, Fmoc-D-Phe-OH for LW01025/Fmoc-D-2-Nal-OH for LW01029, Fmoc-4-amino-(1-carboxymethyl) piperidine, and DOTA(^{t}Bu)₃ were sequentially coupled to the Fmoc-Rink amide-MBHA resin. After being cleaved with TFA/TIS/water/DODT/thioanisole/phenol 81.5:1:5:2.5:5:5 and precipitated with diethyl ether, the LW01025 crude product was purified by HPLC (C18 semi-prep column; flow rate: 4.5 mL/min; 23% ACN and 0.1% TFA in water; retention time=11.3 min) to obtain a white powder (30% yield). ESI-MS: calculated [M+2H]²⁺ for LW01025 C₇₄H₁₀₈N₂₀O₁₈S 799.4; found 799.6. For LW01029, the crude was purified by HPLC (C18 semi-prep column; flow rate: 4.5 mL/min; 25% ACN and 0.1% TFA in water; retention time=13.0 min) and lyophilized to obtain a white powder (38% yield). ESI-MS: calculated [M+2H]²⁺ for LW01029 C₇₈H₁₁₀N₂₀O₁₈S 824.41; found 824.92.

### Synthesis of LW01107, LW01108, LW01110, and LW01142

The chemical structures of LW01107, LW01108, LW01110, and LW01142 are shown below:

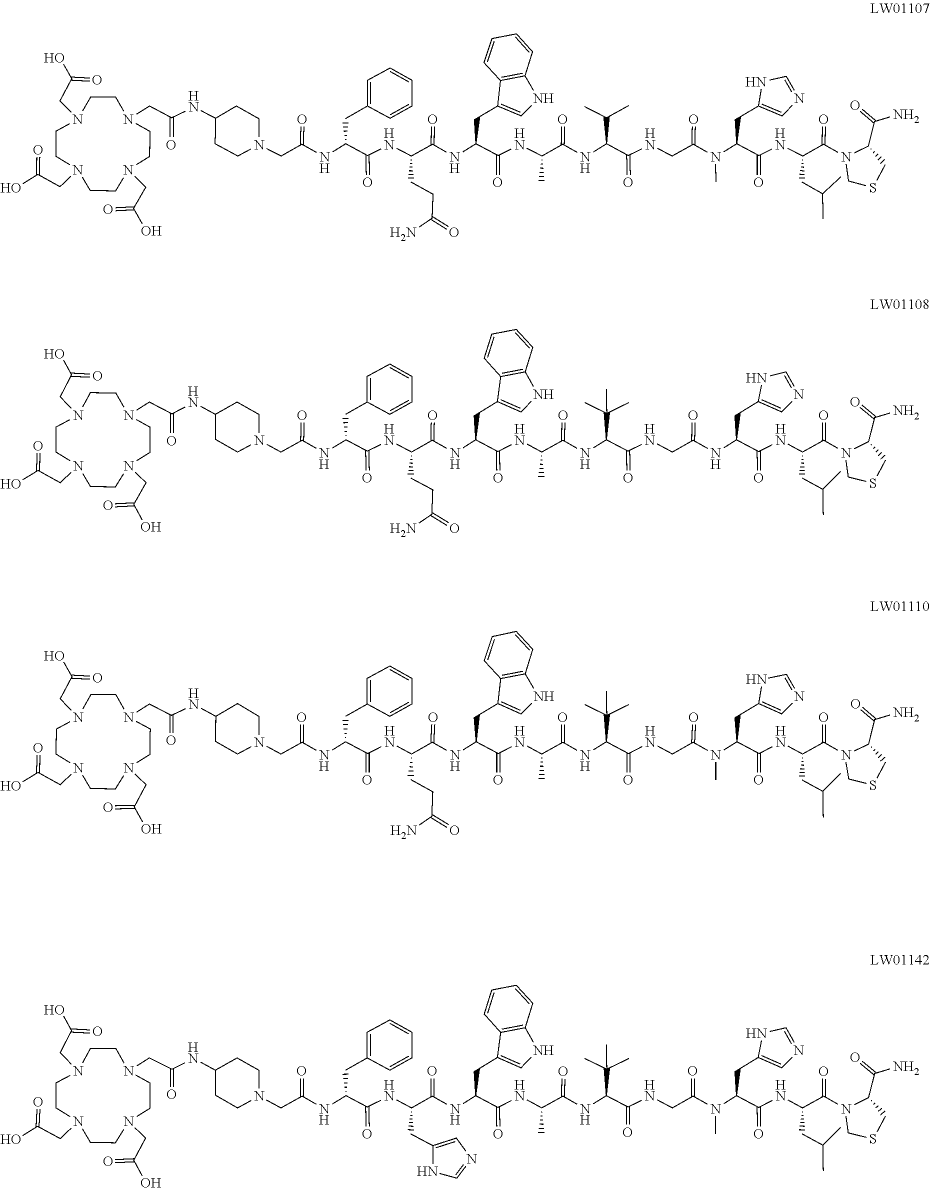

LW01107 (DOTA-Pip-D-Phe-Gln-Trp-Ala-Val-Gly-NMe-His-Leu-Thz-NH₂), LW01108 (DOTA-Pip-D-Phe-Gln-Trp-Ala-Tle-Gly-His-Leu-Thz-NH₂), LW01110 (DOTA-Pip-D-Phe-Gln-Trp-Ala-Tle-Gly-NMe-His-Leu-Thz-NH₂), and LW01142 (DOTA-Pip-D-Phe-His-Trp-Ala-Tle-Gly-NMe-His-Leu-Thz-NH₂) were synthesized using standard Fmoc solid phase synthesis. Fmoc-protected amino acids, Fmoc-4-amino-(1-carboxymethyl) piperidine and DOTA(^{t}Bu)₃ were sequentially coupled to the Fmoc-Rink amide-MBHA resin. After being cleaved with TFA/TIS/water/DODT/thioanisole/phenol 81.5:1:5:2.5:5:5 and precipitated by diethyl ether, the crude products were purified with HPLC (C18 semi-prep column; flow rate: 4.5 mL/min) and lyophilized to give white powders.

For LW01107, the HPLC condition was 23% ACN and 0.1% TFA in water (retention time=14.2 min); yield: 19%. ESI-MS: calculated [M+2H]²⁺ for LW01107 C₇₅H₁₁₀N₂₀O₁₈S 806.41; found 806.80. For LW01108, the HPLC condition was 24% ACN and 0.1% TFA in water (retention time=10.9 min); yield: 26%. ESI-MS: calculated [M+2H]²⁺ for LW01108 C₇₅H₁₁₀N₂₀O₁₈S 806.41; found 807.00. For LW01110, the HPLC condition was 24% ACN and 0.1% TFA in water (retention time=14.9 min); yield: 11%. ESI-MS: calculated [M+2H]²⁺ for LW01110 C₇₆H₁₁₂N₂₀O₁₈S 813.41; found 813.66. For LW01142, the HPLC condition was 25% ACN and 0.1% TFA in water (retention time=12.4 min); yield: 17%. ESI-MS: calculated [M+2H]²⁺ for LW01142 C₇₇H₁₁₁N₂₁O₁₇S 817.92; found 817.88.

### Synthesis of LW01102 and LW01158

The chemical structures of LW01102 and LW01158 are shown below:

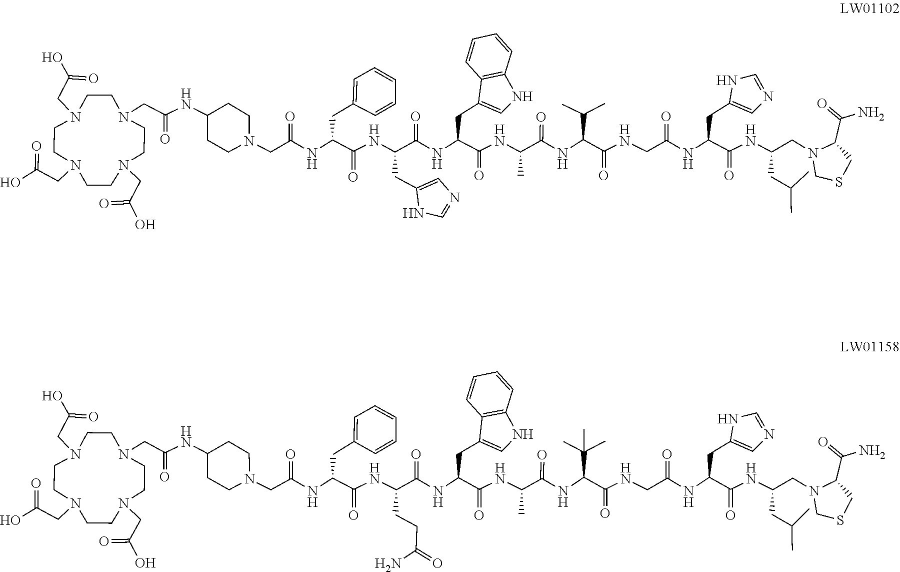 [SMILES: CC(C)C[C@@H](CN1CSC[C@H]1C(N)=O)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](Cc1cnc[nH]1)NC(=O)[C@@H](Cc1ccccc1)NC(=O)CN1CCC(NC(=O)CN2CCN(CC(=O)O)CCN(CC(=O)O)CCN(CC(=O)O)CC2)CC1)C(C)C ; CC(C)C[C@@H](CN1CSC[C@H]1C(N)=O)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)CN1CCC(NC(=O)CN2CCN(CC(=O)O)CCN(CC(=O)O)CCN(CC(=O)O)CC2)CC1)C(C)(C)C]

LW01102 (DOTA-Pip-D-Phe-His-Trp-Ala-Val-Gly-His-LeuψThz-NH₂) and LW01158 (DOTA-Pip-D-Phe-Gln-Trp-Ala-Tle-Gly-His-LeuψThz-NH₂), wherein y is a reduced peptide bond, was synthesized using standard Fmoc solid phase synthesis strategy starting from Sieber resin. Fmoc-LeuψThz-OH (4), Fmoc-protected amino acids, Fmoc-4-amino-(1-carboxymethyl)-piperidine and DOTA(^{t}Bu)₃ were sequentially coupled to the resin. After being cleaved with TFA/TIS/water/DODT/thioanisole/phenol 81.5:1:5:2.5:5:5) and precipitated with diethyl ether, the crude product was purified by HPLC (C18 semi-prep column; flow rate: 4.5 mL/min) and lyophilized to give a white powder.

For LW01102, the HPLC condition was 23.5% ACN and 0.1% TFA in water; (retention time=14.9 min) yield: 35%. ESI-MS: calculated [M+2H]²⁺ for LW01102 C₇₅H₁₆₉N₂₁O₁₆S 796.91; found 796.51. For LW01158, the HPLC condition was 26% ACN and 0.1% TFA in water (retention time=14.1 min); yield: 32%. ESI-MS: calculated [M+2H]²⁺ for LW01158 C₇₅H₁₁₂N₂₀O₁₇S 779.42; found 779.46.

### Example 2

### Synthesis of LW01186, LW02002, LW02021, LW02023 and LW02025

The chemical structures of LW01186, LW02002, LW02021, LW02023, and LW02025 are as follows:

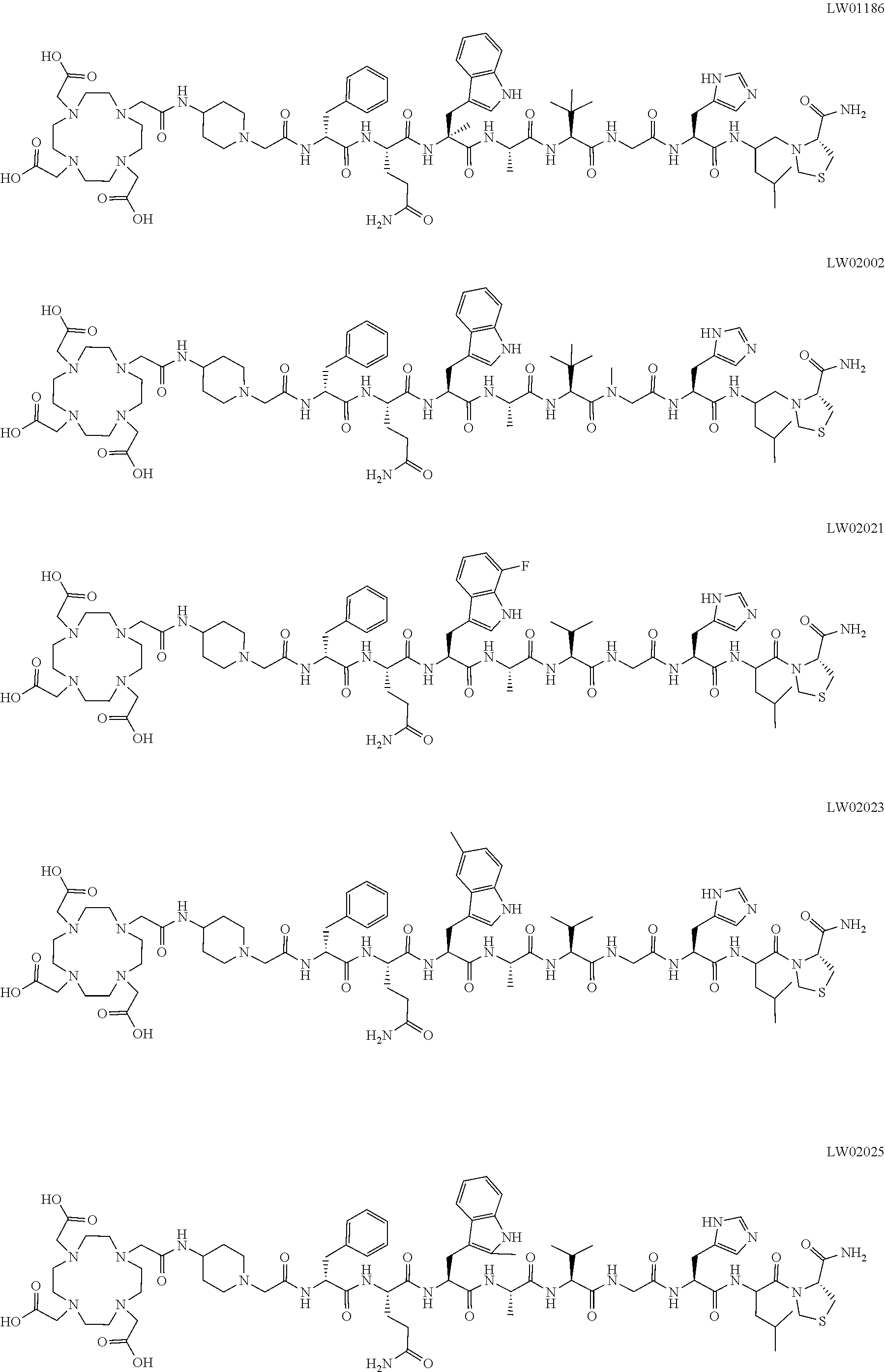

LW01186 (DOTA-Pip-D-Phe-Gln-αMe-Trp-Ala-Tle-Gly-His-LeuψThz-NH₂) and LW02002 (DOTA-Pip-D-Phe-Gln-Trp-Ala-Tle-N-Me-Gly-His-LeuψThz-NH₂), wherein y is a reduced peptide bond, were synthesized using the Fmoc solid phase synthesis strategy starting from Sieber resin. As described above in Example 1, the compound Fmoc-LeuψThz-OH (4), Fmoc-protected amino acids, Fmoc-4-amino-(1-carboxymethyl)-piperidine and DOTA(^{t}Bu)₃ were sequentially coupled to the resin. After being cleaved with TFA/TIS/water/DODT/thioanisole/phenol 81.5:1:5:2.5:5:5) and precipitated with diethyl ether, the crude product was purified by HPLC (018 semi-prep column; flow rate: 4.5 mL/min) and lyophilized to give a white powder.

LW02021 (DOTA-Pip-D-Phe-Gln-7-F-Trp-Ala-Val-Gly-His-Leu-Thz-NH₂), LW02023 (DOTA-Pip-D-Phe-Gln-5-Me-Trp-Ala-Val-Gly-His-Leu-Thz-NH) and LW02025 (DOTA-Pip-D-Phe-Gln-2-Me-Trp-Ala-Val-Gly-His-Leu-Thz-NH₂) were synthesized using the Fmoc solid phase synthesis strategy starting from Fmoc-Rink MBHA resin. Fmoc-protected amino acids, Fmoc-4-amino-(1-carboxymethyl)-piperidine and DOTA(^{t}Bu)₃ were sequentially coupled to the resin. After being cleaved with TFA/TIS/water/DODT/thioanisole/phenol 81.5:1:5:2.5:5:5) and precipitated with diethyl ether, the crude product was purified by HPLC (018 semi-prep column; flow rate: 4.5 mL/min) and lyophilized to give a white powder. The HPLC conditions and results are shown below in Table 5.

TABLE 5
HPLC CONDITIONS AND RESULTS

| Compound | | Retention | Yield | Calculated | Found |
|---|---|---|---|---|---|
| name | HPLC conditions | time (min) | (%) | mass (m/z) | (m/z) |
| LW01186 | 29% CH₃CN and 0.1% | 12.1 | 7.7 | [M + 2H]²⁺ | [M + 2H]²⁺ |
| | TFA in H₂O | | | 806.5 | 806.8 |
| LW02002 | 27% CH₃CN and 0.1% | 15.3 | 17 | [M + 2H]²⁺ | [M + 2H]²⁺ |
| | TFA in H₂O | | | 806.5 | 806.7 |
| LW02021 | 25% CH₃CN and 0.1% | 8.9 | 33 | [M + 2H]²⁺ | [M + 2H]²⁺ |
| | TFA in H₂O | | | 808.4 | 808.4 |
| LW02023 | 25% CH₃CN and 0.1% | 12.2 | 19 | [M + 2H]²⁺ | [M + 2H]²⁺ |
| | TFA in H₂O | | | 806.4 | 806.5 |
| LW02025 | 23% CH₃CN and 0.1% | 13.7 | 16 | [M + 2H]²⁺ | [M + 2H]²⁺ |
| | TFA in H₂O | | | 806.4 | 806.6 |

### Synthesis of LW01080, LW01085, LW01088, and LW01136

The chemical structures of LW01080, LW01085, LW01088 and LW01136 are shown below:

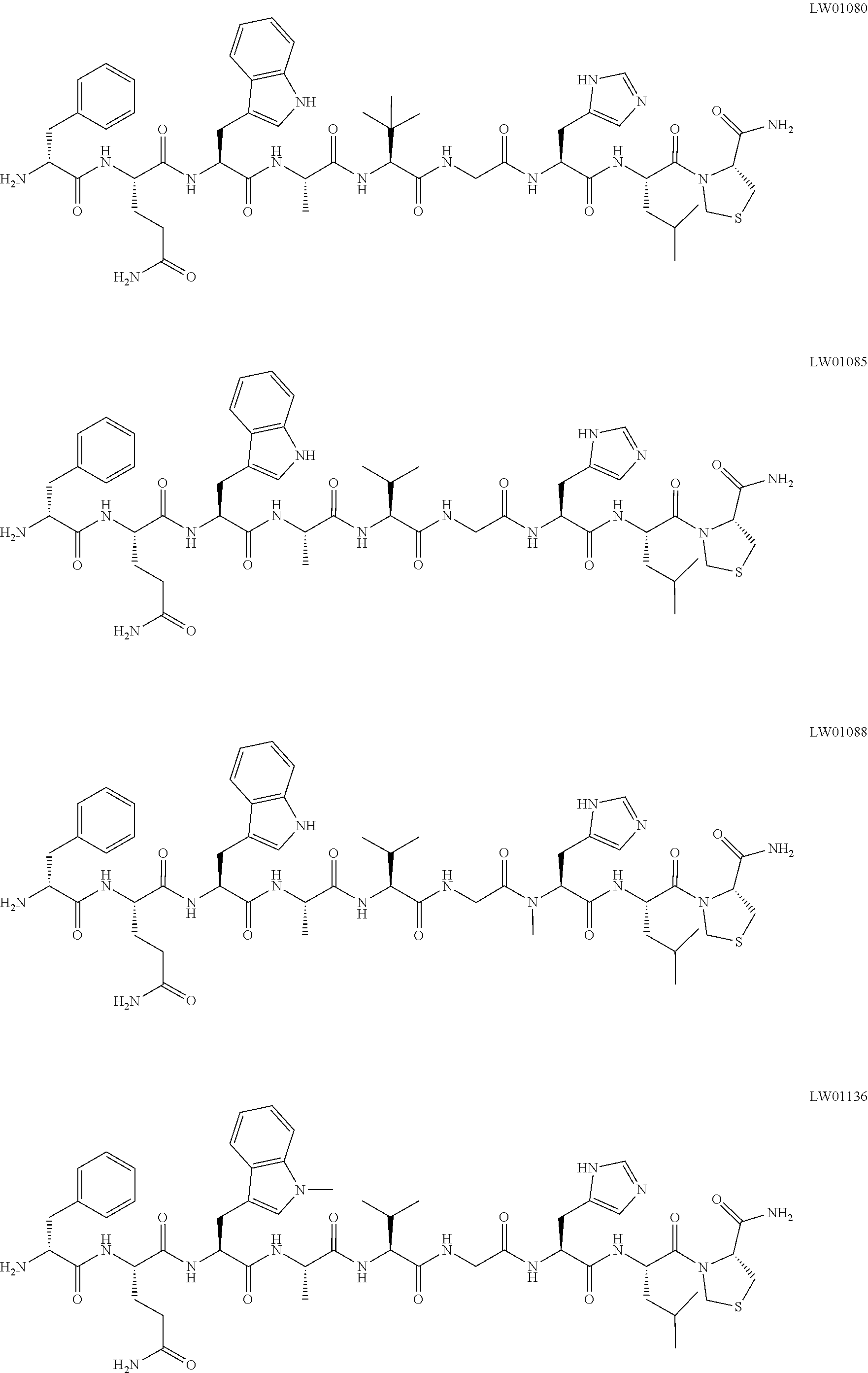

LW01080 (D-Phe-Gln-Trp-Ala-Tle-Gly-His-Leu-Thz-NH₂), LW01085 (D-Phe-Gln-Trp-Ala-Val-Gly-His-Leu-Thz-NH₂), LW01088 (D-Phe-Gln-Trp-Ala-Val-Gly-NMe-His-Leu-Thz-NH₂), and LW01136 (D-Phe-Gln-Trp(Me)-Ala-Val-Gly-His-Leu-Thz-NH₂) were synthesized using standard Fmoc solid phase synthesis. Fmoc-protected amino acids were sequentially coupled to the Fmoc-Rink amide-MBHA resin. After being cleaved with TFA/TIS/water/DODT/thioanisole/phenol 81.5:1:5:2.5:5:5 and precipitated with diethyl ether, the crude products were purified with HPLC (C18 semi-prep column; flow rate: 4.5 mL/min) and lyophilized to give white powders.

For LW01080, the HPLC condition was 26% ACN and 0.1% TFA in water (retention time=9.02 min); yield: 26%. ESI-MS: calculated [M+H]⁺ for LW01080 C₅₂H₇₂N₁₄O₁₀S 1085.54; found 1085.99. For LW01085, the HPLC condition was 23% ACN and 0.1% TFA in water (retention time=16.0 min); yield: 41%. ESI-MS: calculated [M+H]⁺ for LW01085 C₅₁H₇₀N₁₄O₁₀S 1071.52; found 1071.77. For LW01088, the HPLC condition was 23% ACN and 0.1% TFA in water (retention time=17.4 min); yield: 29%. ESI-MS: calculated [M+H]⁺ for LW01088 C₅₂H₇₂N₁₄O₁₀S 1085.54; found 1085.69. For LW01136, the HPLC condition was 27% ACN and 0.1% TFA in water (retention time=13.4 min); yield: 30%. ESI-MS: calculated [M+H]⁺ for LW01136 C₅₂H₇₂N₁₄O₁₀S 1085.54; found 1085.79.

### Example 3

### Synthesis of LW02011, LW02016, LW02019, LW01166, LW01171, LW01173, LW01175, LW01177, LW01180, LW01182, LW01183, LW01191, LW02007, LW02009, LW02013, and LW02015

The chemical structures of LW02011, LW02016, LW02019, LW01166, LW01171, LW01173, LW01175, LW01177, LW01180, LW01182, LW01183, LW01191, LW02007, LW02009, LW02013 and LW02015 are as follows:

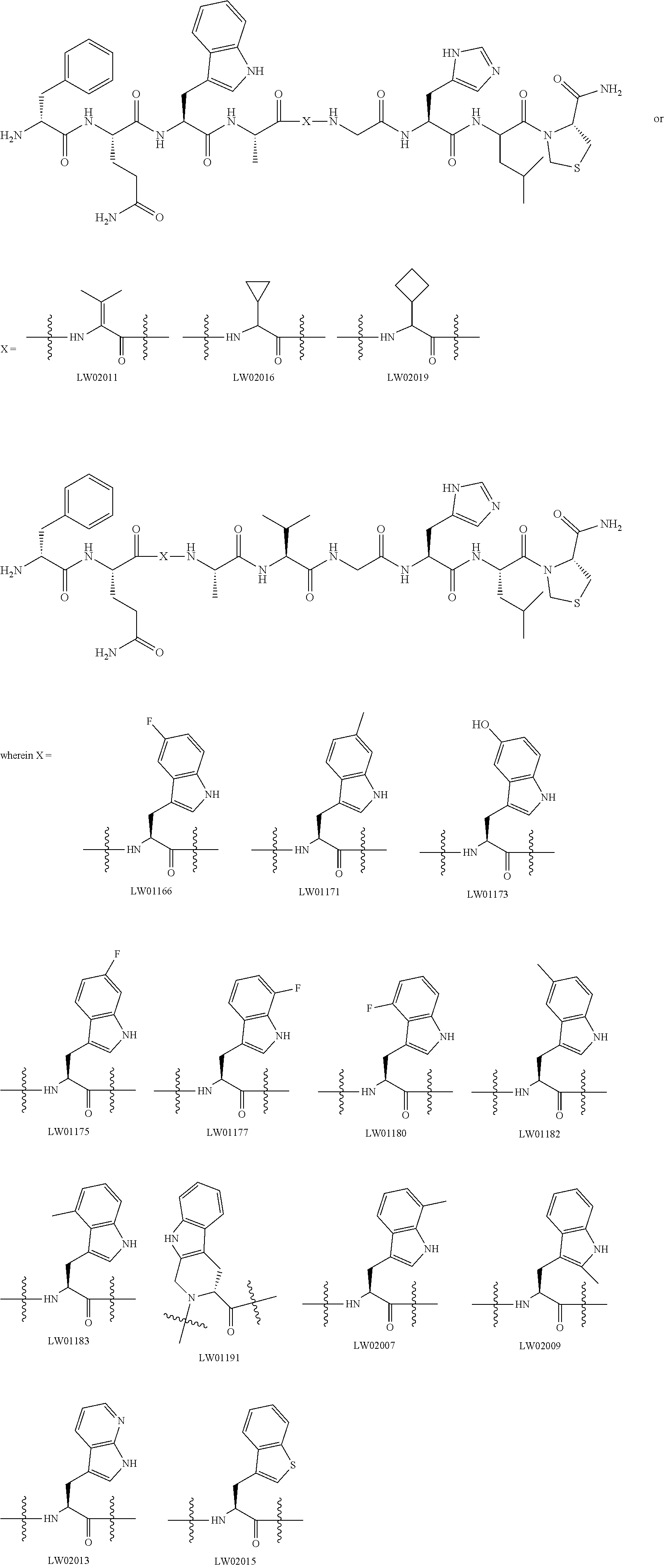

LW02011 (D-Phe-Gln-Trp-Ala-2,3-dehydro-Val-Gly-His-Leu-Thz-NH₂), LW02016 (D-Phe-Gln-Trp-Ala-L-cyclopropylglycine-Gly-His-Leu-Thz-NH₂), LW02019 (D-Phe-Gln-Trp-Ala-cyclobutylglycine-Gly-His-Leu-Thz-NH₂), LW01166 (D-Phe-Gln-5-F-Trp-Ala-Val-Gly-His-Leu-Thz-NH₂), LW01171 (D-Phe-Gln-6-Me-Trp-Ala-Val-Gly-His-Leu-Thz-NH₂), LW01173 (D-Phe-Gln-5-OH-Trp-Ala-Val-Gly-His-Leu-Thz-NH₂), LW01175 (D-Phe-Gln-6-F-Trp-Ala-Val-Gly-His-Leu-Thz-NH₂), LW01177 (D-Phe-Gln-7-F-Trp-Ala-Val-Gly-His-Leu-Thz-NH₂), LW01180 (D-Phe-Gln-4-F-Trp-Ala-Val-Gly-His-Leu-Thz-NH₂), LW01182 (D-Phe-Gln-5-Me-Trp-Ala-Val-Gly-His-Leu-Thz-NH₂), LW01183 (D-Phe-Gln-4-Me-Trp-Ala-Val-Gly-His-Leu-Thz-NH₂), LW01191 (D-Phe-Gln-D-Tpi-Ala-Val-Gly-His-Leu-Thz-NH₂), LW02007 (D-Phe-Gln-7-Me-Trp-Ala-Val-Gly-His-Leu-Thz-NH₂), LW02009 (D-Phe-Gln-2-Me-Trp-Ala-Val-Gly-His-Leu-Thz-NH₂), LW02013 (D-Phe-Gln-7-Aza-Trp-Ala-Val-Gly-His-Leu-Thz-NH₂), and LW02015 (D-Phe-Gln-Bta-Ala-Val-Gly-His-Leu-Thz-NH₂) were synthesized using standard Fmoc solid phase synthesis. Fmoc-protected amino acids were sequentially coupled to the Fmoc-Rink amide-MBHA resin. After being cleaved with TFA/TIS/water/DODT/thioanisole/phenol 81.5:1:5:2.5:5:5 and precipitated with diethyl ether, the crude products were purified with HPLC (C18 semi-prep column; flow rate: 4.5 mL/min) and lyophilized to give white powders. The HPLC conditions are shown below in Table 6.

TABLE 6
HPLC CONDITIONS AND RESULTS

| Compound | | Retention | Yield | Calculated | Found |
|---|---|---|---|---|---|
| name | HPLC conditions | time (min) | (%) | mass (m/z) | (m/z) |
| LW02011 | 25% CH₃CN and 0.1% | 9.0 | 5.0 | [M + H]⁺ | [M + H]⁺ |
| | TFA in H₂O | | | 1069.5 | 1069.7 |
| LW02016 | 25% CH₃CN and 0.1% | 8.4 | 11 | [M + H]⁺ | [M + H]⁺ |
| | TFA in H₂O | | | 1069.5 | 1069.4 |
| LW02019 | 26% CH₃CN and 0.1% | 9.8 | 15 | [M + H]⁺ | [M + H]⁺ |
| | TFA in H₂O | | | 1083.5 | 1083.5 |
| LW01166 | 25% CH₃CN and 0.1% | 10.5 | 46 | [M + H]⁺ | [M + H]⁺ |
| | TFA in H₂O | | | 1089.5 | 1089.5 |
| LW01171 | 26% CH₃CN and 0.1% | 11.5 | 29 | [M + H]⁺ | [M + H]⁺ |
| | TFA in H₂O | | | 1085.5 | 1085.7 |
| LW01173 | 22% CH₃CN and 0.1% | 8.6 | 21 | [M + H]⁺ | [M + H]⁺ |
| | TFA in H₂O | | | 1087.5 | 1087.6 |
| LW01175 | 25% CH₃CN and 0.1% | 13.0 | 40 | [M + H]⁺ | [M + H]⁺ |
| | TFA in H₂O | | | 1089.5 | 1089.6 |
| LW01177 | 25% CH₃CN and 0.1% | 12.2 | 24 | [M + H]⁺ | [M + H]⁺ |
| | TFA in H₂O | | | 1089.5 | 1089.7 |
| LW01180 | 25% CH₃CN and 0.1% | 11.4 | 36 | [M + H]⁺ | [M + H]⁺ |
| | TFA in H₂O | | | 1089.5 | 1089.5 |
| LW01182 | 26% CH₃CN and 0.1% | 11.7 | 33 | [M + H]⁺ | [M + H]⁺ |
| | TFA in H₂O | | | 1085.5 | 1085.6 |
| LW01183 | 26% CH₃CN and 0.1% | 10.3 | 26 | [M + H]⁺ | [M + H]⁺ |
| | TFA in H₂O | | | 1085.5 | 1085.7 |
| LW01191 | 27% CH₃CN and 0.1% | 11.3 | 20 | [M + H]⁺ | [M + H]⁺ |
| | TFA in H₂O | | | 1083.5 | 1083.5 |
| LW02007 | 27% CH₃CN and 0.1% | 9.0 | 25 | [M + H]⁺ | [M + H]⁺ |
| | TFA in H₂O | | | 1085.5 | 1085.5 |
| LW02009 | 26% CH₃CN and 0.1% | 9.1 | 23 | [M + H]⁺ | [M + H]⁺ |
| | TFA in H₂O | | | 1085.5 | 1085.5 |
| LW02013 | 18% CH₃CN and 0.1% | 13.5 | 8.4 | [M + H]⁺ | [M + H]⁺ |
| | TFA in H₂O | | | 1072.5 | 1072.5 |
| LW02015 | 26% CH₃CN and 0.1% | 14.4 | 24 | [M + H]⁺ | [M + H]⁺ |
| | TFA in H₂O | | | 1088.5 | 1088.4 |

### Example 4

### Syntheses of Nonradioactive Ga-Complexed Standards of LW01025, LW01029, LW01107, LW01108, LW01110, LW01102, LW01142, LW01158, LW01186, LW02002, LW02021, LW02023, and LW02025.

LW01025 (2.82 mg), LW01029 (2.12 mg), LW01107 (2.20 mg), LW01108 (2.42 mg), LW01110 (2.03 mg), LW01102 (2.17 mg), LW01142 (1.81 mg), LW01158 (2.54 mg) were dissolved respectively in 0.5 mL NaOAc buffer (0.1 N, pH 4.53) and GaCl₃ (5 eq., 0.2 M) was added. Approximately 2 mg of LW01186, LW02002, LW02021, LW02023, and LW02025 were dissolved each in 0.5 mL NaOAc buffer (0.1 N, pH 4.53) and GaCl₃ (5 eq., 0.2 M) was added. The respective reaction mixtures were incubated at 80° C. for 15 min and then purified with HPLC (018 semi-prep column) and lyophilized to give white powders. The HPLC conditions are shown below in Table 7.

TABLE 7
HPLC CONDITIONS AND RESULTS

| Compound | | Retention | Yield | Calculated | Found |
|---|---|---|---|---|---|
| name | HPLC conditions | time (min) | (%) | mass (m/z) | (m/z) |
| Ga-LW01025 | 23% CH₃CN and 0.1% | 12.0 | 82 | [M + 2H]²⁺ | [M + 2H]²⁺ |
| | TFA in H₂O | | | 833.9 | 833.7 |
| Ga-LW01029 | 25% CH₃CN and 0.1% | 13.9 | 63 | [M + 2H]²⁺ | [M + 2H]²⁺ |
| | TFA in H₂O | | | 858.9 | 858.5 |
| Ga-LW01107 | 23% CH₃CN and 0.1% | 14.2 | 73 | [M + 2H]²⁺ | [M + 2H]²⁺ |
| | TFA in H₂O | | | 840.9 | 840.4 |
| Ga-LW01108 | 24% CH₃CN and 0.1% | 13.2 | 59 | [M + 2H]²⁺ | [M + 2H]²⁺ |
| | TFA in H₂O | | | 840.9 | 840.9 |
| Ga-LW01110 | 24% CH₃CN and 0.1% | 15.8 | 54 | [M + 2H]²⁺ | [M + 2H]²⁺ |
| | TFA in H₂O | | | 847.9 | 847.8 |
| Ga-LW01102 | 23.5% CH₃CN and 0.1% | 15.7 | 43 | [M + 2H]²⁺ | [M + 2H]²⁺ |
| | TFA in H₂O | | | 831.4 | 831.4 |
| Ga-LW01142 | 25% CH₃CN and 0.1% | 11.1 | 53 | [M + 2H]²⁺ | [M + 2H]²⁺ |
| | TFA in H₂O | | | 851.4 | 851.9 |
| Ga-LW01158 | 26% CH₃CN and 0.1% | 13.2 | 76 | [M + 2H]²⁺ | [M + 2H]²⁺ |
| | TFA in H₂O | | | 832.9 | 832.9 |
| Ga-LW01186 | 29% CH₃CN and 0.1% | 13.5 | 81 | [M + 2H]²⁺ | [M + 2H]²⁺ |
| | TFA in H₂O | | | 839.4 | 839.5 |
| Ga-LW02002 | 27% CH₃CN and 0.1% | 16.3 | 77 | [M + 2H]²⁺ | [M + 2H]²⁺ |
| | TFA in H₂O | | | 839.4 | 839.4 |
| Ga-LW02021 | 25% CH₃CN and 0.1% | 14.4 | 82 | [M + 2H]²⁺ | [M + 2H]²⁺ |
| | TFA in H₂O | | | 841.4 | 841.5 |
| Ga-LW02023 | 25% CH₃CN and 0.1% | 13.1 | 84 | [M + 2H]²⁺ | [M + 2H]²⁺ |
| | TFA in H₂O | | | 839.4 | 839.8 |
| Ga-LW02025 | 23% CH₃CN and 0.1% | 16.8 | 81 | [M + 2H]²⁺ | [M + 2H]²⁺ |
| | TFA in H₂O | | | 839.4 | 832.9 |

### Example 5

### Syntheses of Nonradioactive Lu-Complexed Standards of LW01090, LW01110, and LW01142

Approximately 2 mg of LW01090, LW01110, or LW01142 were dissolved respectively in 0.5 mL NaOAc buffer (0.1 N, pH 4.48) and LuCl₃ (10 eq., 0.1 M) was added. The reaction mixture was incubated at 80° C. for 30 min and then purified with HPLC (C18 semi-prep column) and lyophilized to give white powders. The HPLC conditions are shown below in Table 8.

TABLE 8
HPLC CONDITIONS AND RESULTS

| | | Reten- | | | |
|---|---|---|---|---|---|
| | | tion | | Calculated | |
| Compound | HPLC | time | Yield | mass | Found |
| name | conditions | (min) | (%) | (m/z) | (m/z) |
| Lu- | 26% CH₃CN | 13.9 | 47 | [M + 2H]²⁺ | [M + 2H]²⁺ |
| LW01090 | and 0.1% | | | 885.4 | 885.6 |
| | TFA in H₂O | | | | |
| Lu- | 24% CH₃CN | 18.0 | 88 | [M + 2H]²⁺ | [M + 2H]²⁺ |
| LW01110 | and 0.1% | | | 899.4 | 899.7 |
| | TFA in H₂O | | | | |
| Lu- | 24% CH₃CN | 15.9 | 61 | [M + 2H]²⁺ | [M + 2H]²⁺ |
| LW01142 | and 0.1% | | | 903.9 | 904.0 |
| | TFA in H₂O | | | | |

### Example 6

### Synthesis of ⁶⁸Ga-Labeled Peptides

Purified ⁶⁸Ga in 0.5 mL water was added into a 4-mL glass vial preloaded with 0.7 mL of HEPES buffer (2 M, pH 5.0) and 10 μL precursor solution (1 mM). The radiolabeling reaction was carried out under microwave heating for 1 min before being purified by HPLC using the semi-preparative column. The eluate fraction containing the radiolabeled product was collected, diluted with water (50 mL), and passed through a C18 Sep-Pak cartridge that was pre-washed with ethanol (10 mL) and water (10 mL). After washing the C18 Sep-Pak cartridge with water (10 mL), the ⁶⁸Ga-labeled product was eluted off the cartridge with ethanol (0.4 mL), and diluted with saline for imaging and biodistribution. Quality control was performed using the analytical column. The tracers were obtained with more than 95% radiochemical purity.

### In Vitro Competition Binding Assay

PC-3 cells were seeded at 2×10⁵ cells/well in 24-well poly-D-lysine plates 24-48 hours prior to the experiment. The growth medium was replaced by 400 μL of reaction medium (RPMI 1640 containing 2 mg/mL BSA, 4.8 mg/mL HEPES, 1 U/mL penicillin G and 1 μg/mL streptomycin). Cells were incubated for 30-60 min at 37° C. Peptides as provided in Table 9 below provided in 50 μL of decreasing concentrations (10 μM to 1 pM) and 50 μL of 0.011 nM [¹²⁵I-Tyr⁴]bombesin were added to wells. The cells were incubated with moderate agitation for 1 h at 27° C., washed twice with ice-cold PBS, harvested by trypsinization, and measured for radioactivity on the gamma counter. Data were analyzed using nonlinear regression (one binding site model for competition assay) with GraphPad Prism 8.

TABLE 9
Binding affinities (Ki, nM) of GRPR-targeting peptides
Binding affinities Ki (n = 2-3)

| Compound | Mean | SD |
|---|---|---|
| LW01080 | 3.56 | 0.72 |
| LW01085 | 8.77 | 1.33 |
| LW01088 | 9.00 | 1.99 |
| LW01136 | 35.7 | 13.8 |
| LW02016 | 202 | 103 |
| LW02019 | 35.7 | 27.4 |
| LW01166 | 17.9 | 0.57 |
| LW01175 | 46.9 | 9.73 |
| LW01177 | 9.38 | 1.68 |
| LW01180 | 43.4 | 10.2 |
| LW01182 | 10.5 | 1.86 |
| LW01183 | 25.9 | 4.51 |
| LW02007 | 13.5 | 1.79 |
| LW02009 | 11.5 | 1.71 |
| LW02013 | 166 | 73.4 |
| LW02015 | 53.1 | 28.2 |
| Ga-LW01025 | 7.62 | 0.19 |
| Ga-LW01029 | 6.07 | 0.59 |
| Ga-LW01107 | 2.98 | 0.60 |
| Ga-LW01108 | 1.34 | 0.12 |
| Ga-LW01110 | 1.39 | 0.03 |
| Ga-LW01102 | 2.84 | 1.01 |
| Ga-LW01142 | 3.45 | 0.90 |
| Ga-LW01158 | 5.35 | 0.27 |
| Ga-LW01186 | 6.94 | 0.95 |
| Ga-LW02002 | 11.0 | 0.39 |
| Ga-LW02021 | 13.6 | 2.83 |
| Ga-LW02023 | 14.9 | 3.39 |
| Ga-LW02025 | 14.7 | 4.20 |
| Lu-LW01090 | 12.6 | 1.02 |
| Lu-LW01110 | 3.07 | 0.15 |
| Lu-LW01142 | 2.37 | 0.28 |

### PET/CT Imaging and Ex Vivo Biodistribution in PC-3 Tumor-Bearing Mice

All imaging and biodistribution studies were performed using male NOD.Cg-Rag1^{tm1Mom} Il2rg^{tm1Wjl}/SzJ (NRG) mice and conducted according to the guidelines established by the Canadian Council on Animal Care and approved by Animal Ethics Committee of the University of British Columbia. For tumor inoculations, mice were anesthetized by inhalation with 2% isoflurane in oxygen and implanted subcutaneously with 5×10⁶ PC-3 cells below the left shoulder. Imaging and biodistribution studies were performed only after tumors grew to 5-8 mm in diameter.

For PET/CT imaging studies, ˜3-4 MBq of the ⁶⁸Ga-labeled tracer was injected through the tail vein. Mice were allowed to recover and roam freely in the cages after injecting the tracer. At 45 min post-injection (p.i.), mice were sedated again and positioned on the scanner. First, a 10 min CT scan was conducted for localization and attenuation correction for reconstruction of PET images, before a 10 min PET image was acquired. Heating pads were used during the entire procedure to keep the mice warm. For ex vivo biodistribution studies, mice were injected with ˜1.5-3 MBq of the ⁶⁸Ga-labeled tracer. At 1 h p.i., mice were euthanized, blood was drawn from heart, and organs/tissues of interest were collected, rinsed with PBS, blotted dry, weighed, and counted using an automated gamma counter. The uptake in each organ/tissue was normalized to the injected dose and expressed as the percentage of the injected dose per gram of tissue (% ID/g).

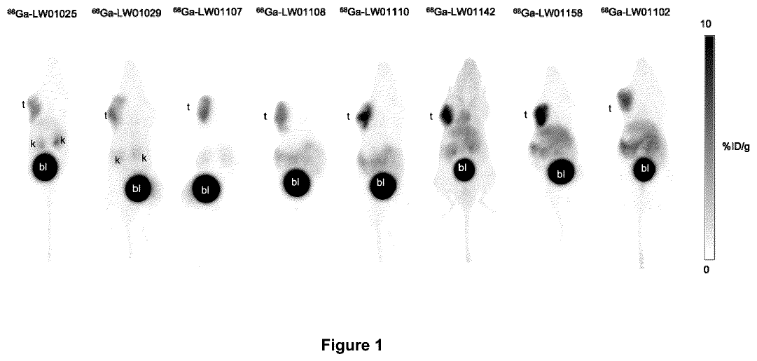

A representative maximum-intensity-projection PET image of ⁶⁸Ga-LW01025, ⁶⁸Ga-LW01029, ⁶⁸Ga-LW01107, ⁶⁸Ga-LW01108, ⁶⁸Ga-LW01110, ⁶⁸Ga-LW01142, ⁶⁸Ga-LW01158, and ⁶⁸Ga-LW01102 in mice bearing PC-3 tumor xenografts is shown in FIG. 1. Biodistribution data is shown in Tables 10 and 11.

TABLE 10
Biodistribution data (at 1 h post-injection, %
ID/g) of ⁶⁸Ga-LW01025, ⁶⁸Ga-LW01108, and ⁶⁸Ga-LW01110
in mice bearing PC-3 tumor xenografts.

⁶⁸Ga-LW01025 (n = 4)

| Tissues | Mean | SD |
|---|---|---|
| PC3 tumor | 6.20 | 0.14 |
| Blood | 0.34 | 0.07 |
| Fat | 0.09 | 0.02 |
| Seminal | 0.06 | 0.02 |
| Testes | 0.16 | 0.06 |
| Small Intestine | 0.56 | 0.12 |
| Large Intestine | — | — |
| Spleen | 0.23 | 0.06 |
| Pancreas | 1.28 | 0.16 |
| Stomach | 0.54 | 0.33 |
| Liver | 0.29 | 0.13 |
| Adrenal | 0.54 | 0.13 |
| kidneys | 2.37 | 0.12 |
| Heart | 0.13 | 0.01 |
| Lungs | 0.34 | 0.05 |
| Bone | 0.24 | 0.12 |
| Muscle | 0.13 | 0.10 |
| Brain | 0.03 | 0.01 |

Table 11 shows Biodistribution data of ⁶⁸Ga-LW01029, ⁶⁸Ga-LW01107, ⁶⁸Ga-LW01142, ⁶⁸Ga-LW01158, and ⁶⁸Ga-LW01102 and further completed distribution data of ⁶⁸Ga-LW01108 and ⁶⁸Ga-LW01110 in mice bearing PC-3 tumor xenografts.

TABLE 11
Biodistribution data (at 1 h post injection, %
ID/g) in mice bearing PC-3 tumor xenografts.

| | n = 4 | | | | | | | |
|---|---|---|---|---|---|---|---|---|
| | ⁶⁸Ga- | | ⁶⁸Ga- | | ⁶⁸Ga- | | ⁶⁸Ga- | |
| | LW01029 | | LW01107 | | LW01108 | | LW01110 | |
| Tissues | Mean | SD | Mean | SD | Mean | SD | Mean | SD |
| PC3 | 4.70 | 1.27 | 7.05 | 0.71 | 5.90 | 0.68 | 16.59 | 1.60 |
| tumor | | | | | | | | |
| Blood | 1.08 | 0.47 | 0.30 | 0.04 | 0.59 | 0.18 | 0.69 | 0.14 |
| Fat | 0.18 | 0.19 | 0.05 | 0.01 | 0.06 | 0.02 | 0.09 | 0.02 |
| Seminal | 0.13 | 0.07 | 0.04 | 0.01 | 0.11 | 0.03 | 0.10 | 0.06 |
| Testes | 0.33 | 0.13 | 0.10 | 0.02 | 0.16 | 0.03 | 0.16 | 0.07 |
| Small | 1.05 | 0.30 | 0.31 | 0.01 | 2.29 | 0.54 | 2.11 | 0.48 |
| Intestine | | | | | | | | |
| Large | 0.96 | 0.42 | 0.18 | 0.04 | 1.31 | 0.33 | 1.27 | 0.27 |
| Intestine | | | | | | | | |
| Spleen | 0.45 | 0.35 | 0.13 | 0.02 | 0.40 | 0.30 | 0.29 | 0.05 |
| Pancreas | 2.56 | 0.55 | 0.39 | 0.03 | 9.32 | 1.97 | 8.99 | 1.54 |
| Stomach | 0.77 | 0.42 | 0.07 | 0.01 | 0.89 | 0.18 | 0.94 | 0.31 |
| Liver | 0.63 | 0.23 | 0.32 | 0.15 | 0.35 | 0.19 | 0.41 | 0.06 |
| Adrenal | 0.82 | 0.45 | 0.32 | 0.19 | 1.51 | 0.99 | 1.64 | 0.18 |
| Kidneys | 4.73 | 2.57 | 1.88 | 0.17 | 2.47 | 0.66 | 3.26 | 0.25 |
| Heart | 0.36 | 0.17 | 0.11 | 0.02 | 0.18 | 0.04 | 0.23 | 0.03 |
| Lungs | 1.82 | 0.59 | 0.28 | 0.04 | 0.49 | 0.22 | 0.59 | 0.12 |
| Bone | 0.19 | 0.09 | 0.06 | 0.02 | 0.15 | 0.11 | 0.12 | 0.00 |
| Muscle | 0.23 | 0.13 | 0.07 | 0.02 | 0.17 | 0.11 | 0.14 | 0.03 |
| Brain | 0.04 | 0.02 | 0.01 | 0.00 | 0.02 | 0.00 | 0.03 | 0.00 |

| | n = 4 | | | | | |
|---|---|---|---|---|---|---|
| | ⁶⁸Ga-LW01142 | | ⁶⁸Ga-LW01158 | | ⁶⁸Ga-LW01102 | |
| Tissues | Mean | SD | Mean | SD | Mean | SD |
| PC3 | 11.43 | 1.22 | 11.15 | 0.65 | 7.46 | 0.44 |
| tumor | | | | | | |
| Blood | 6.88 | 0.29 | 1.14 | 0.15 | 1.13 | 0.25 |
| Fat | 0.32 | 0.08 | 0.07 | 0.01 | 0.09 | 0.03 |
| Seminal | 0.33 | 0.02 | 0.15 | 0.12 | 0.23 | 0.27 |
| Testes | 1.28 | 0.19 | 0.24 | 0.01 | 0.33 | 0.12 |
| Small | 1.70 | 0.10 | 2.46 | 0.30 | 2.85 | 0.39 |
| Intestine | | | | | | |
| Large | 1.07 | 0.09 | 1.41 | 0.38 | 1.25 | 0.27 |
| Intestine | | | | | | |
| Spleen | 0.88 | 0.09 | 0.60 | 0.42 | 0.52 | 0.27 |
| Pancreas | 4.40 | 0.27 | 12.01 | 1.41 | 13.14 | 1.31 |
| Stomach | 0.98 | 0.05 | 1.30 | 0.41 | 1.23 | 0.68 |
| Liver | 2.88 | 0.45 | 4.33 | 0.22 | 3.22 | 0.32 |
| Adrenal | 2.07 | 0.27 | 1.37 | 0.35 | 1.36 | 0.44 |
| Kidneys | 6.36 | 0.41 | 2.98 | 0.34 | 6.30 | 0.47 |
| Heart | 1.62 | 0.12 | 0.34 | 0.05 | 0.36 | 0.05 |
| Lungs | 4.59 | 0.81 | 1.19 | 0.32 | 1.55 | 0.61 |
| Bone | 0.47 | 0.08 | 0.13 | 0.01 | 0.13 | 0.07 |
| Muscle | 0.52 | 0.07 | 0.20 | 0.05 | 0.18 | 0.01 |
| Brain | 0.15 | 0.02 | 0.03 | 0.00 | 0.04 | 0.01 |

In Vivo Stability of ⁶⁸Ga-LW01025, ⁶⁸Ga-LW01029, ⁶⁸Ga-LW01107, ⁶⁸Ga-LW01108, ⁶⁸Ga-LW01110, ⁶⁸Ga-LW01102, and ⁶⁸Ga-LW01142

In vivo plasma stability studies were conducted for ⁶⁸Ga-labeled tracers to evaluate metabolic stability at 15 min p.i. Approximately 5.56 to 15.3 MBq of ⁶⁸Ga-LW01025, ⁶⁸Ga-LW01029, ⁶⁸Ga-LW01107, ⁶⁸Ga-LW01108, ⁶⁸Ga-LW01110, ⁶⁸Ga-LW01102, or ⁶⁸Ga-LW01142 were injected via tail vein into three male NRG mice, respectively. At 15 min p.i., mice were sedated and euthanized and their blood and urine were collected. The plasma was extracted from whole blood with ACN, vortexed, centrifuged, and the supernatant collected. The plasma and urine were analyzed with radio-HPLC (C18 analytical column; flow rate: 2.0 mL/min). The HPLC condition was same as the HPLC condition for quality control.

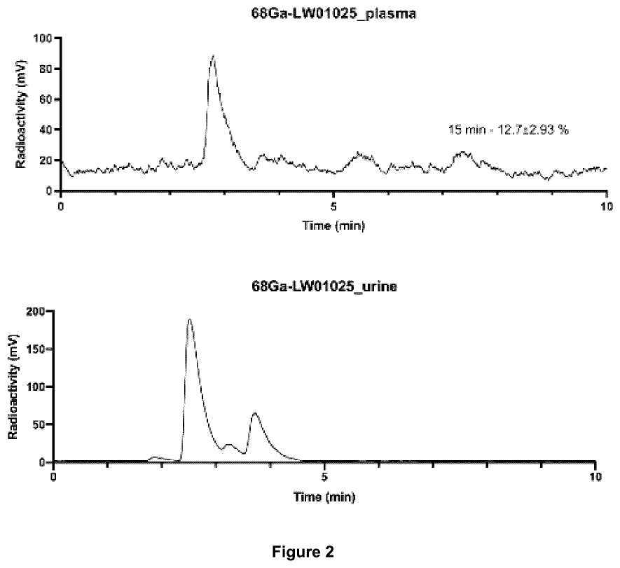

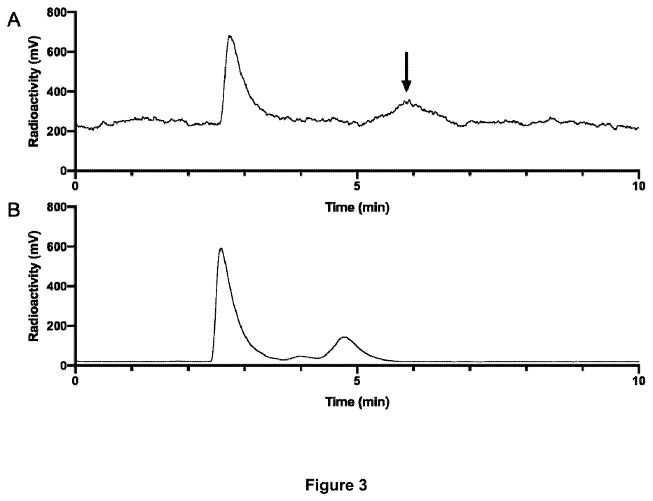

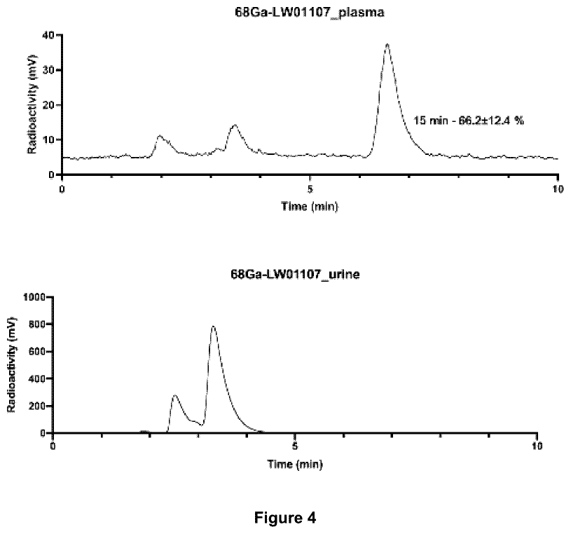

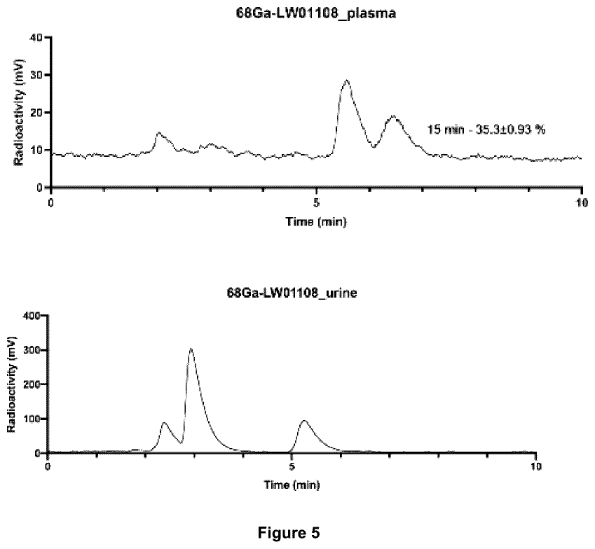

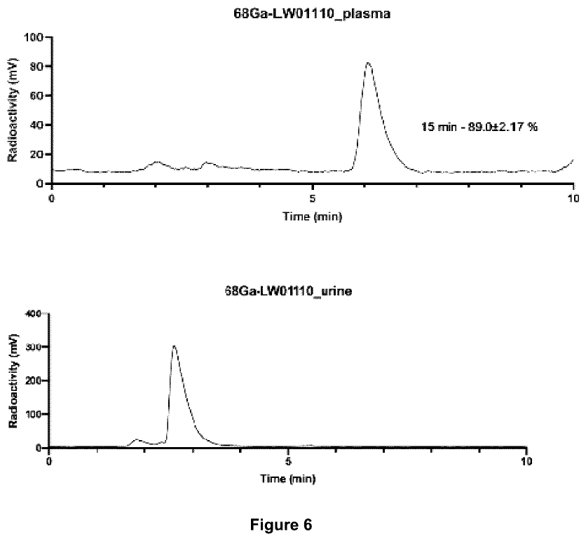

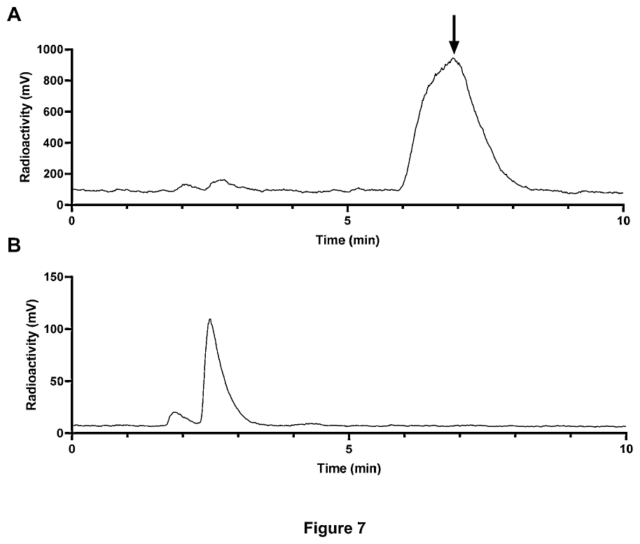

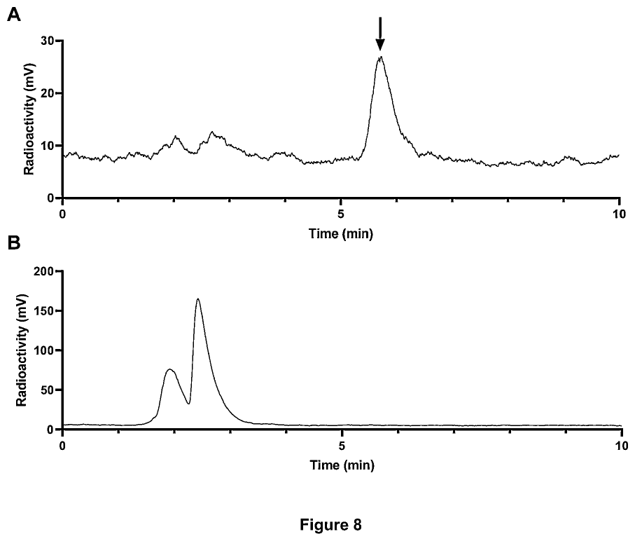

Representative radio-HPLC chromatograms of ⁶⁸Ga-LW01025, ⁶⁸Ga-LW01029, ⁶⁸Ga-LW01107, ⁶⁸Ga-LW01108, ⁶⁸Ga-LW01110, ⁶⁸Ga-LW01102, and ⁶⁸Ga-LW01142 extracted from mouse urine and plasma samples are shown in FIGS. 2-8, respectively.

### Example 7

### Synthesis of LW01045, LW01059, LW01061, LW01090, and LW01117

The chemical structures of LW01045, LW01059, LW01061, LW01090, and LW01117 are as follows:

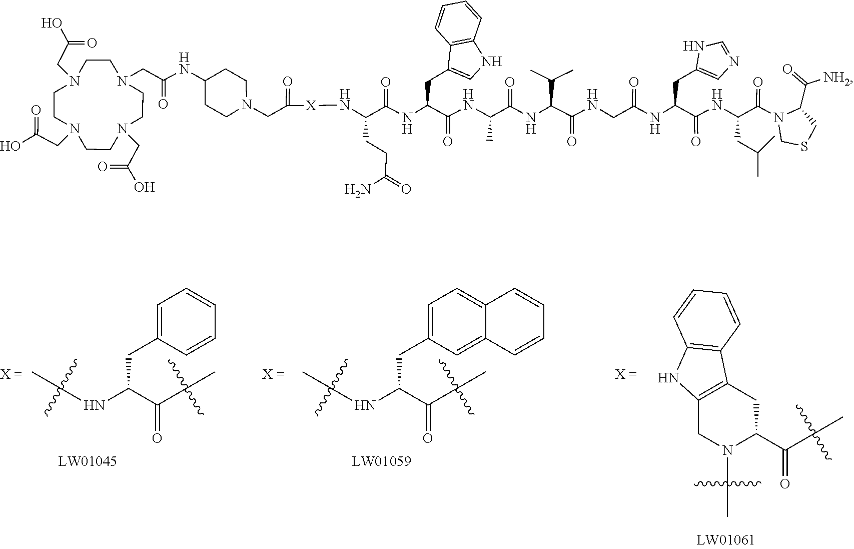

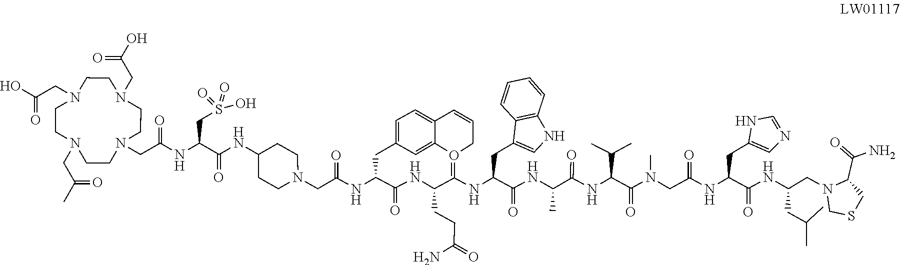

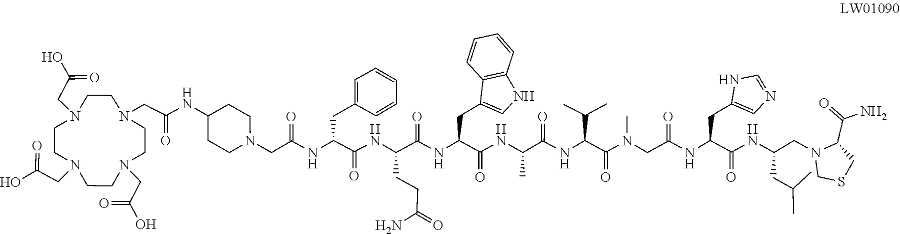 [SMILES: CC(C)C[C@@H](CN1CSC[C@H]1C(N)=O)NC(=O)[C@H](Cc1cnc[nH]1)NC(=O)CN(C)C(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)CN1CCC(NC(=O)CN2CCN(CC(=O)O)CCN(CC(=O)O)CCN(CC(=O)O)CC2)CC1)C(C)C]

### Synthesis of DOTA-Conjugated Peptides LW01045, LW01059, LW01061, LW01090, and LW01117.

LW01045 (DOTA-Pip-D-Phe-Gln-Trp-Ala-Val-Gly-His-Leu-ψ-Thz-NH₂), LW01059 (DOTA-Pip-D-2-Nal-Gln-Trp-Ala-Val-Gly-His-Leu-ψ-Thz-NH₂), LW01061 (DOTA-Pip-D-Tpi-Gln-Trp-Ala-Val-Gly-His-Leu-ψ-Thz-NH₂), LW01090 (DOTA-Pip-D-Phe-Gln-Trp-Ala-Val-NMe-Gly-His-Leu-ψ-Thz-NH₂), and LW01117 (DOTA-Cysteic acid-Pip-D-2-Nal-Gln-Trp-Ala-Val-Gly-His-Leu-ψ-Thz-NH₂) wherein ψ is a reduced peptide bond, were synthesized using the Fmoc solid phase synthesis strategy starting from Sieber resin. As described in Example 1, the compound Fmoc-LeuψThz-OH (4), Fmoc-protected amino acids, Fmoc-4-amino-(1-carboxymethyl)-piperidine and DOTA(^{t}Bu)₃ were sequentially coupled to the resin. After being cleaved with TFA/TIS/water/DODT/thioanisole/phenol 81.5:1:5:2.5:5:5) and precipitated with diethyl ether, the crude product was purified by HPLC (C18 semi-prep column; flow rate: 4.5 mL/min) and lyophilized. The HPLC conditions, retention times, isolated yields, and MS confirmations of DOTA-conjugated peptides are provided in Table 12.

TABLE 12
HPLC Purification conditions and MS characterizations
of LW01045, LW01059, LW01061, LW01090, and LW01117

| Compound | | Retention | Yield | Calculated | Found |
|---|---|---|---|---|---|
| name | HPLC conditions | time (min) | (%) | mass (m/z) | (m/z) |
| LW01045 | 25% CH₃CN and 0.1% | 18.7 | 34 | [M + 2H]²⁺ | [M + 2H]²⁺ |
| | TFA in H₂O | | | 792.4 | 792.8 |
| LW01059 | 28% CH₃CN and 0.1% | 15.8 | 49 | [M + 2H]²⁺ | [M + 2H]²⁺ |
| | TFA in H₂O | | | 817.4 | 817.8 |
| LW01061 | 28% CH₃CN and 0.1% | 16.6 | 38 | [M + 2H]²⁺ | [M + 2H]²⁺ |
| | TFA in H₂O | | | 817.9 | 818.2 |
| LW01090 | 25% CH₃CN and 0.1% | 18.3 | 32 | [M + 2H]²⁺ | [M + 2H]²⁺ |
| | TFA in H₂O | | | 799.4 | 799.7 |
| LW01117 | 29% CH₃CN and 0.1% | 14.6 | 14 | [M + 2H]²⁺ | [M + 2H]²⁺ |
| | TFA in H₂O | | | 892.9 | 893.4 |

### Synthesis of Nonradioactive Ga-Complexed Standards of LW01045, LW01059, LW01061, LW01090, and LW01117

Non-radioactive Ga-complexed standards of LW01045, LW01059, LW01061, LW01090, and LW01117 were prepared according to the procedure set forth in Example 1. Briefly, LW01045, LW01059, LW01061, LW01090, and LW01117 were mixed and incubated with 0.5 mL NaOAc buffer (0.1 N, pH 4.2-4.5) and GaCl₃ (5 eq., 0.2 M) at 80° C. for 15 min and then purified with HPLC (C18 semi-prep column) and lyophilized. The HPLC conditions, retention times, isolated yields and MS confirmations of these non-radioactive Ga-complexed standards are provided in Table 13.

TABLE 13
HPLC purification conditions and MS characterizations of Ga-
complexed LW01045, LW01059, LW01061, LW01090, and LW01117

| | | Retention | Yield | Calculated | Found |
|---|---|---|---|---|---|
| Compound name | HPLC conditions | time (min) | (%) | mass (m/z) | (m/z) |
| Ga-LW01045 | 26% CH₃CN and 0.1% | 12.5 | 72 | [M + 2H]²⁺ | [M + 2H]²⁺ |
| | TFA in H₂O | | | 825.9 | 826.0 |
| Ga-LW01059 | 28% CH₃CN and 0.1% | 18.3 | 67 | [M + 2H]²⁺ | [M + 2H]²⁺ |
| | TFA in H₂O | | | 850.9 | 850.7 |
| Ga-LW01061 | 28% CH₃CN and 0.1% | 18.8 | 68 | [M + 2H]²⁺ | [M + 2H]²⁺ |
| | TFA in H₂O | | | 851.4 | 851.2 |
| Ga-LW01090 | 25% CH₃CN and 0.1% | 18.3 | 75 | [M + 2H]²⁺ | [M + 2H]²⁺ |
| | TFA in H₂O | | | 832.9 | 832.8 |
| Ga-LW01117 | 29% CH₃CN and 0.1% | 14.6 | 57 | [M + 2H]²⁺ | [M + 2H]²⁺ |
| | TFA in H₂O | | | 926.4 | 926.4 |

### Synthesis of ⁶⁸Ga-Labeled Compounds

Radiolabeled LW01045, LW01059, LW01090, and LW01117 were prepared according to the procedure set forth in Example 1. Briefly, purified ⁶⁸Ga in 0.5 mL water was added into a 4-mL glass vial preloaded with 0.7 mL of HEPES buffer (2 M, pH 5.0) and 10 μL precursor solution (1 mM). The radiolabeling reaction was carried out under microwave heating for 1 min before being purified by HPLC using the semi-preparative column. The eluate fraction containing the radiolabeled product was collected, diluted with water (50 mL), and passed through a C18 Sep-Pak cartridge that was pre-washed with ethanol (10 mL) and water (10 mL). After washing the C18 Sep-Pak cartridge with water (10 mL), the ⁶⁸Ga-labeled product was eluted off the cartridge with ethanol (0.4 mL), and diluted with saline for imaging and biodistribution. Quality control was performed using the analytical column. The tracers were obtained with more than 95% radiochemical purity. The HPLC conditions and retention times are provided in Table 14. The tracers were obtained in 42-59% decay-corrected radiochemical yields with >66 GB/pmol molar activity and >92% radiochemical purity.

TABLE 14
HPLC Conditions for the purification and quality control
of ⁶⁸Ga-labeled LW01045, LW01059, LW01090, and LW01117.

| Compound | | | Retention |
|---|---|---|---|
| name | HPLC conditions | | time (min) |
| [⁶⁸Ga]Ga- | Semi- | 20% CH₃CN and 0.1% FA in H₂O; | 12.6 |
| LW01045 | Prep | flow rate 4.5 mL/min | |
| | QC | 23% CH₃CN and 0.1% FA in H₂O; | 7.2 |
| | | flow rate 2.0 mL/min | |
| [⁶⁸Ga]Ga- | Semi- | 21% CH₃CN and 0.1% FA in H₂O; | 35.8 |
| LW01059 | Prep | flow rate 4.5 mL/min | |
| | QC | 26% CH₃CN and 0.1% FA in H₂O; | 8.8 |
| | | flow rate 2 mL/min | |
| [⁶⁸Ga]Ga- | Semi- | 20% CH₃CN and 0.1% FA in H₂O; | 10.7 |
| LW01090 | Prep | flow rate 4.5 mL/min | |
| | QC | 23% CH₃CN and 0.1% FA in H₂O; | 5.1 |
| | | flow rate 2.0 mL/min | |
| [⁶⁸Ga]Ga- | Semi- | 29% CH₃CN and 0.1% TFA in H₂O; | 14.6 |
| LW01117 | Prep | flow rate 4.5 mL/min | |
| | QC | 28% CH₃CN and 0.1% FA in H₂O; | 6.7 |
| | | flow rate 2.0 mL/min | |

### In Vitro Competition Binding Assay

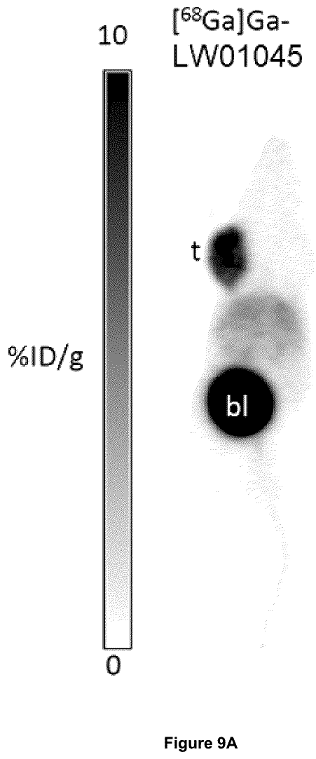

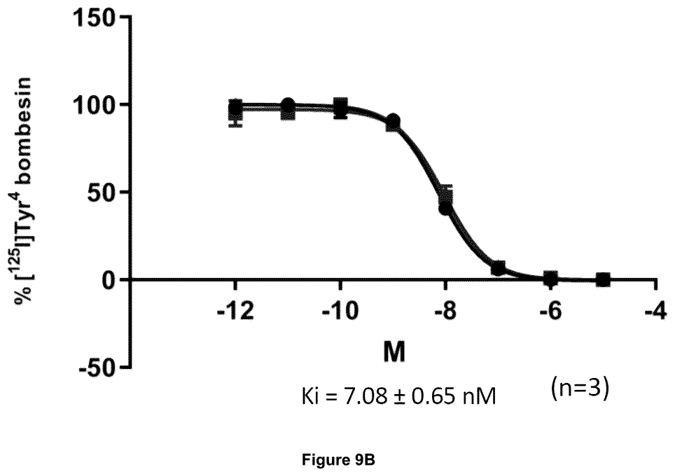

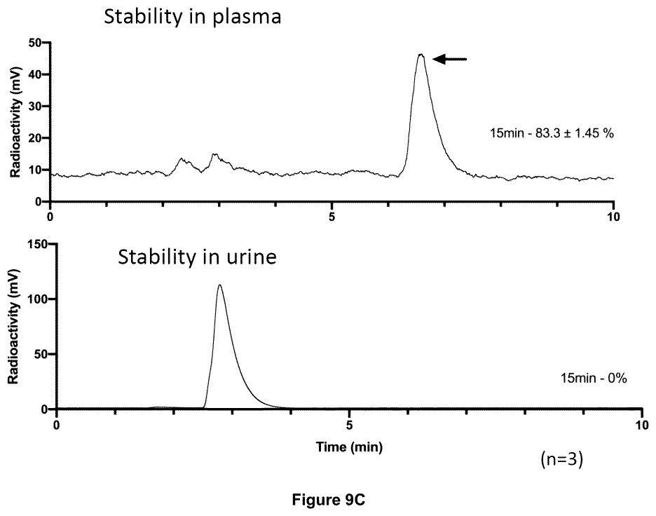

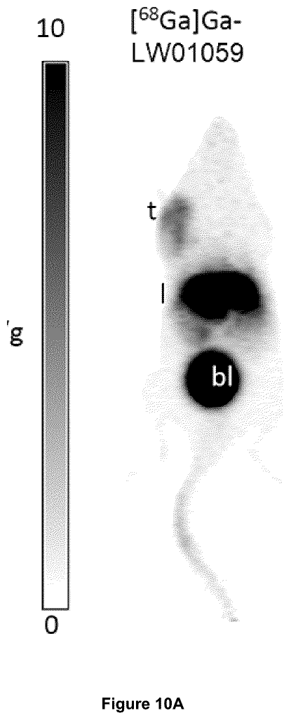

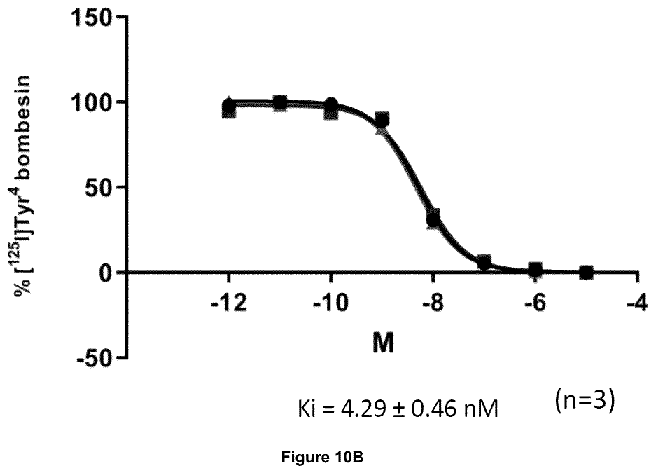

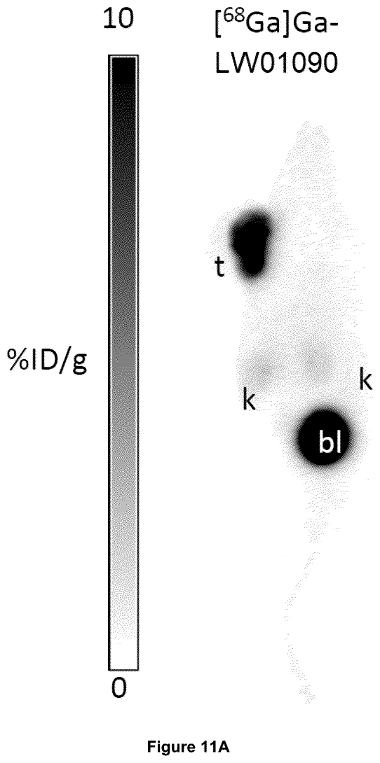

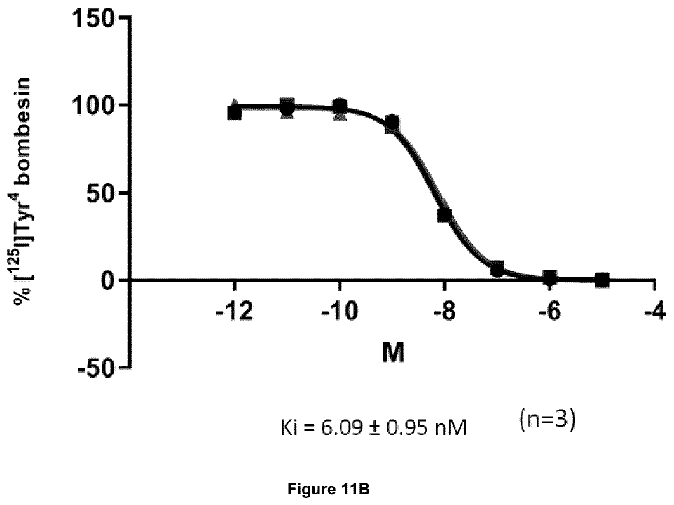

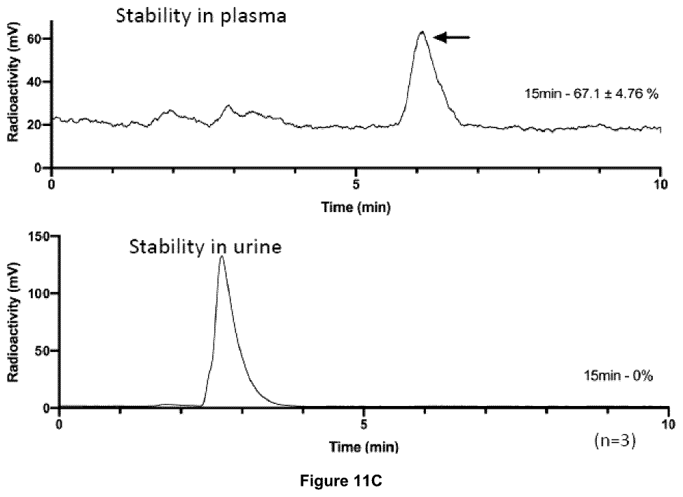

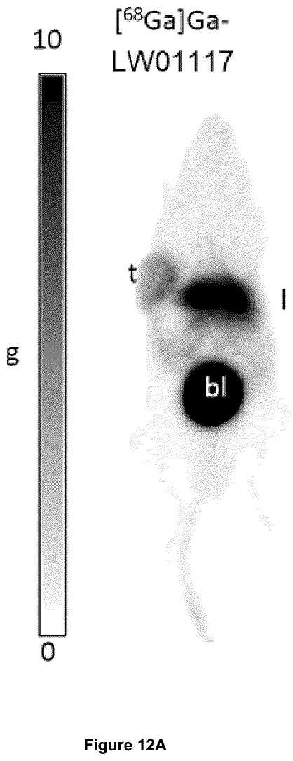

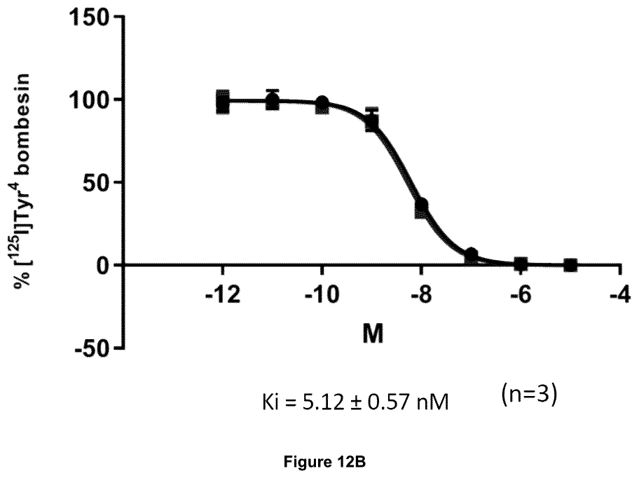

In vitro competition binding assays were performed according to the procedure outlined in Example 1. Specifically, the binding affinities of Ga-LW01045, Ga-LW01059, Ga-LW01090, and Ga-LW01117 were measured by a cell-based binding assay using GRPR-expressing PC-3 prostate cancer cells. Ga-LW01045, Ga-LW01059, Ga-LW01090, and Ga-LW01117 inhibited the binding of [¹²⁵I-Tyr⁴]Bombesin in a dose dependent manner (FIGS. 9B, 10B, 11B, and 12B, respectively). The calculated Ki values of Ga-LW01045, Ga-LW01059, Ga-LW01090, and Ga-LW01117 are enumerated in Table 15.

TABLE 15
Binding affinities (Ki, nM) of GRPR-targeting peptides.
Binding affinities Ki (n = 2-3)

| Compound | Mean | SD |
|---|---|---|
| LW01045 | 7.08 | 0.65 |
| LW01059 | 4.29 | 0.46 |
| LW01061 | >400 | — |
| LW01090 | 6.09 | 0.95 |
| LW01117 | 5.12 | 0.57 |

### PET/CT Imaging and Ex Vivo Biodistribution in PC-3 Tumor-Bearing Mice

All imaging and biodistribution studies were performed using the procedure outlined in Example 1. The PC-3 tumor xenografts clearly visualized in PET images acquired at 1 h post-injection using ⁶⁸Ga-LW01045, ⁶⁸Ga-LW0159, ⁶⁸Ga-LW01090, and ⁶⁸Ga-LW0117 (FIGS. 9A, 10A, 11A, and 12A, respectively and Table 16).

TABLE 16
Biodistribution data (at 1 h post-injection, % ID/g) of ⁶⁸Ga-LW01045, ⁶⁸Ga-
LW0159, ⁶⁸Ga-LW01090, and ⁶⁸Ga-LW0117 in mice bearing PC-3 tumor xenografts.

| | ⁶⁸Ga-LW01045 | | ⁶⁸Ga-LW01059 | | ⁶⁸Ga-LW01090 | | ⁶⁸Ga-LW01117 | | | |
|---|---|---|---|---|---|---|---|---|---|---|
| | (n = 4) | | (n = 4) | | (n = 4) | | (n = 4) | | | |
| Tissues | Mean | SD | Mean | SD | Mean | SD | Mean | SD | Mean* | SD* |
| Blood | 0.76 | 0.21 | 2.16 | 1.86 | 0.76 | 0.08 | 1.86 | 0.12 | 2.57 | 0.68 |
| Fat | 0.09 | 0.03 | 0.19 | 0.25 | 0.11 | 0.01 | 0.25 | 0.05 | 0.58 | 0.22 |
| Testes | 0.19 | 0.05 | 0.33 | 0.60 | 0.23 | 0.02 | 0.60 | 0.16 | 1.06 | 0.13 |
| Small Intestine | 1.04 | 0.30 | 2.15 | 1.62 | 0.66 | 0.09 | 1.62 | 0.07 | 1.63 | 0.62 |
| Large Intestine | 0.37 | 0.16 | 0.66 | 0.50 | 0.41 | 0.09 | 0.50 | 0.04 | 1.16 | 0.41 |
| Spleen | 0.47 | 0.17 | 0.68 | 0.84 | 0.30 | 0.03 | 0.84 | 0.17 | 0.84 | 0.26 |
| Pancreas | 2.81 | 0.78 | 7.26 | 6.50 | 1.98 | 0.10 | 6.50 | 0.36 | 0.78 | 0.31 |
| Stomach | 0.32 | 0.08 | 1.10 | 0.57 | 0.40 | 0.15 | 0.57 | 0.07 | 0.63 | 0.20 |
| Liver | 2.61 | 0.70 | 21.5 | 12.5 | 0.64 | 0.11 | 12.5 | 0.88 | 1.87 | 0.35 |
| Adrenal Glands | 0.57 | 0.40 | 1.81 | 1.27 | 0.58 | 0.10 | 1.27 | 0.33 | 0.85 | 0.32 |
| kidneys | 2.51 | 0.59 | 4.49 | 3.84 | 3.52 | 0.41 | 3.84 | 0.43 | 22.9 | 9.41 |
| Heart | 0.27 | 0.08 | 0.66 | 0.58 | 0.24 | 0.03 | 0.58 | 0.04 | 0.87 | 0.26 |
| Lungs | 0.75 | 0.52 | 3.05 | 1.95 | 0.55 | 0.07 | 1.95 | 0.96 | 2.13 | 0.61 |
| PC-3 Tumor | 10.2 | 2.27 | 6.84 | 6.63 | 15.7 | 2.17 | 6.63 | 0.40 | 2.60 | 0.42 |
| Bone | 0.19 | 0.06 | 0.42 | 0.26 | 0.10 | 0.04 | 0.26 | 0.07 | 0.70 | 0.35 |
| Muscle | 0.15 | 0.05 | 0.28 | 0.35 | 0.20 | 0.08 | 0.35 | 0.14 | 0.91 | 0.37 |
| Brain | 0.05 | 0.03 | 0.06 | 0.05 | 0.03 | 0.01 | 0.05 | 0.00 | 0.08 | 0.02 |
| Tumor/bone | 61.3 | 25.0 | 17.0 | 27.5 | 175 | 82.4 | 27.5 | 7.98 | 4.37 | 1.99 |
| Tumor/muscle | 70.1 | 14.2 | 26.0 | 20.5 | 82.3 | 19.2 | 20.5 | 6.45 | 3.21 | 1.18 |
| Tumor/blood | 14.0 | 3.48 | 3.28 | 3.58 | 20.6 | 2.96 | 3.58 | 0.24 | 1.05 | 0.25 |
| Tumor/kidney | 4.10 | 0.46 | 1.55 | 1.73 | 4.48 | 0.69 | 1.73 | 0.13 | 0.13 | 0.05 |
| Tumor/pancreas | 3.70 | 0.55 | 0.98 | 1.02 | 7.95 | 1.40 | 1.02 | 0.05 | 3.77 | 1.50 |

*1 h blocked

In Vivo Stability of ⁶⁸Ga-LW01045 and ⁶⁸Ga-LW01090

The in vivo studies were similarly performed according to the procedure set forth in Example 1. For these studies, ⁶⁸Ga-LW01045 and ⁶⁸Ga-LW01090 was injected via the lateral caudal vein into healthy male NRG mice (n=3). At 15 min post-injection, the mice were sedated and euthanized, and urine and blood were collected. The plasma was extracted from whole blood by the addition of CH₃CN (500 L), vortexing, centrifugation, and the separation of the supernatants. The plasma and urine samples were analyzed via radio-HPLC by using the conditions for quality control of these ⁶⁸Ga-labeled radioligands. FIGS. 90 and 110 show that ⁶⁸Ga-LW01045 and ⁶⁸Ga-LW01090 were sufficiently stable in vivo in NRG mice with 83.3±1.45% and 67.1±4.76% remaining intact in plasma post-injection. The present invention has been described with regard to one or more embodiments.

### Example 8

### Synthesis of LW02045 and LW02042

The chemical structures of LW02045 and LW02042 are as follows:

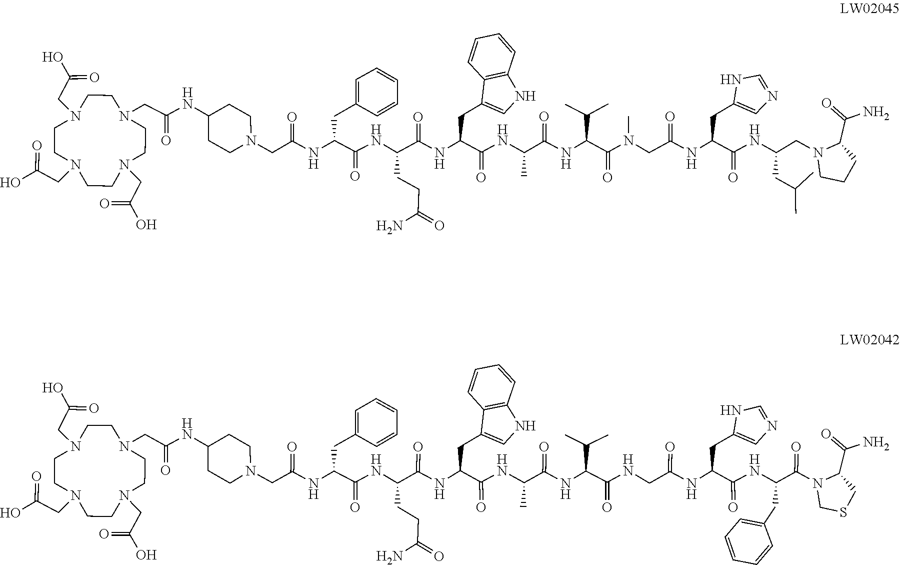 [SMILES: CC(C)C[C@@H](CN1CCC[C@H]1C(N)=O)NC(=O)[C@H](Cc1cnc[nH]1)NC(=O)CN(C)C(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)CN1CCC(NC(=O)CN2CCN(CC(=O)O)CCN(CC(=O)O)CCN(CC(=O)O)CC2)CC1)C(C)C ; CC(C)[C@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)CN1CCC(NC(=O)CN2CCN(CC(=O)O)CCN(CC(=O)O)CCN(CC(=O)O)CC2)CC1)C(=O)NCC(=O)N[C@@H](Cc1cnc[nH]1)C(=O)N[C@@H](Cc1ccccc1)C(=O)N1CSC[C@H]1C(N)=O]

### Synthesis of DOTA-Conjugated LW02045 and LW02042

LW02045 (DOTA-Pip-D-Phe-Gln-Trp-Ala-Val-N-MeGly-His-LeuψPro-NH₂), wherein ψ is a reduced peptide bond, was synthesized using standard Fmoc solid phase synthesis strategy starting from Sieber resin. Fmoc-LeuψPro-OH, Fmoc-protected amino acids, Fmoc-4-amino-(1-carboxymethyl)-piperidine and DOTA(tBu)₃ were sequentially coupled to the resin. After being cleaved with TFA/TIS/water/DODT/thioanisole/phenol 81.5:1:5:2.5:5:5) and precipitated with diethyl ether, the crude product was purified by HPLC (C18 semi-prep column; flow rate: 4.5 mL/min) and lyophilized to give a white powder. The HPLC condition was 20% ACN and 0.1% TFA in water (retention time=20.1 min); yield: 37%. ESI-MS: calculated [M+2H]2+ for LW02045 C₇₆H₁₁₄N₂₀O₁₇S 790.44; found 790.50.

LW02042 (DOTA-Pip-D-Phe-Gln-Trp-Ala-Val-Gly-His-Phe-Thz-NH₂) was synthesized using standard Fmoc solid phase synthesis. Fmoc-protected amino acids, Fmoc-4-amino-(1-carboxymethyl) piperidine and DOTA(tBu)₃ were sequentially coupled to the Fmoc-Rink amide-MBHA resin. After being cleaved with TFA/TIS/water/DODT/thioanisole/phenol 81.5:1:5:2.5:5:5 and precipitated by diethyl ether, the crude products were purified with HPLC (C18 semi-prep column; flow rate: 4.5 mL/min) and lyophilized to give white powders. The HPLC condition was 23% ACN and 0.1% TFA in water (retention time=11.4 min); yield: 31%. ESI-MS: calculated [M+2H]2+ for LW02042 C₇₇H₁₁₆N₂₀O₁₈S 816.40; found 816.44.

### Syntheses of Nonradioactive Ga-Complexed Standards of LW02045 and LW02042

LW02045 (2.85 mg) and LW02042 (2.91 mg) were dissolved respectively in 0.5 mL NaOAc buffer (0.1 N, pH 4.48) and GaCl₃ (5 eq., 0.2 M) was added. The reaction mixture was incubated at 80° C. for 15 min and then purified with HPLC (C18 semi-prep column) and lyophilized to give white powders.

For Ga-LW02045, the HPLC condition was 20% ACN and 0.1% TFA in water at a flow rate of 4.5 mL/min (retention time=23.6 min); yield: 74%. ESI-MS: calculated [M+2H]2+ for Ga-LW02045 C₇₆H₁₁₂GaN₂₀O₁₇S 823.90; found 823.86. For Ga-LW02042, the HPLC condition was 23% ACN and 0.1% TFA in water at a flow rate of 4.5 mL/min (retention time=18.9 min); yield: 91%. ESI-MS: calculated [M+2H]2+ for Ga-LW02042 C₇₇H₁₀₄GaN₂₀O₁₈S 849.85; found 849.63.

### PET/CT Imaging and Ex Vivo Biodistribution in PC-3 Tumor-Bearing Mice

All imaging and biodistribution studies were performed using male NOD.Cg-Rag1tm1Mom Il2rgtm1Wjl/SzJ (NRG) mice and conducted according to the guidelines established by the Canadian Council on Animal Care and approved by Animal Ethics Committee of the University of British Columbia. For tumor inoculations, mice were anesthetized by inhalation with 2% isoflurane in oxygen and implanted subcutaneously with 5×106 PC-3 cells below the left shoulder. Imaging and biodistribution studies were performed only after tumors grew to 5-8 mm in diameter.

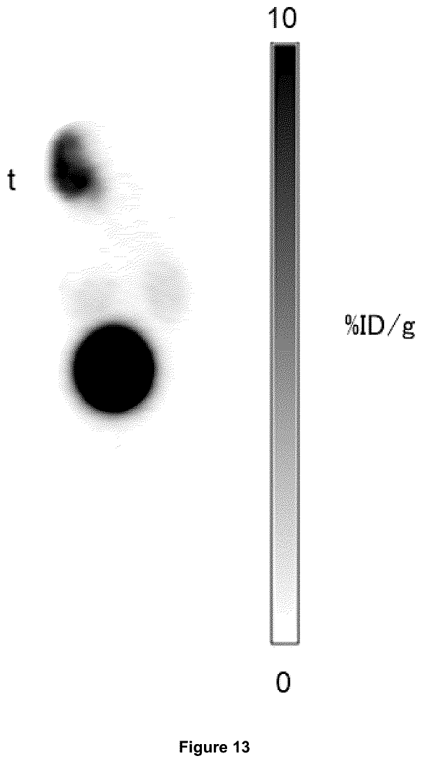

For PET/CT imaging studies, ˜5 MBq of the ⁶⁸Ga-labeled tracer was injected through the tail vein. Mice were allowed to recover and roam freely in the cages after injecting the tracer. At 45 min post-injection (p.i.), mice were sedated again and positioned on the scanner. First, a 10 min CT scan was conducted for localization and attenuation correction for reconstruction of PET images, before a 10 min PET image was acquired. Heating pads were used during the entire procedure to keep the mice warm. For ex vivo biodistribution studies, mice were injected with ˜3 MBq of the 68Ga-labeled tracer. At 1 h p.i., mice were euthanized, blood was drawn from heart, and organs/tissues of interest were collected, rinsed with PBS, blotted dry, weighed, and counted using an automated gamma counter. The uptake in each organ/tissue was normalized to the injected dose and expressed as the percentage of the injected dose per gram of tissue (% ID/g) (FIG. 13 and Table 17).

TABLE 17
Biodistribution data (at 1 h post-injection, % ID/g) of
⁶⁸Ga-LW02045 in mice bearing PC-3 tumor xenografts.
⁶⁸Ga-LW02045 (n = 4)

| Tissues | Mean | SD |
|---|---|---|
| PC-3 tumor | 12.44 | 1.35 |
| Blood | 0.43 | 0.09 |
| Fat | 0.03 | 0.01 |
| Seminal | 3.80 | 7.44 |
| Testes | 0.12 | 0.02 |
| Small Intestine | 0.48 | 0.14 |
| Large Intestine | 0.34 | 0.16 |
| Spleen | 0.18 | 0.04 |
| Pancreas | 1.37 | 0.40 |
| Stomach | 0.35 | 0.17 |
| Liver | 0.37 | 0.07 |
| Adrenal | 0.44 | 0.15 |
| Kidneys | 2.54 | 0.54 |
| Heart | 0.14 | 0.02 |
| Lungs | 1.08 | 0.16 |
| Bone | 0.07 | 0.01 |
| Muscle | 0.10 | 0.01 |
| Brain | 0.02 | 0.00 |

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes. It will be apparent to persons skilled in the art that a number of variations and modifications can be made without departing from the scope of the invention as defined in the following claims. The scope of the invention should therefore not be limited by the preferred embodiments set forth in the above Examples, but should be given the broadest interpretation consistent with the description as a whole.

What is claimed is:

1. A peptidic compound, wherein the compound has the structure of Formula A or is a salt or solvate of Formula A,
R^{rad}ₙ₆-[linker]-R^{L}-Xaa¹-Xaa²-Xaa³-Xaa⁴-Xaa⁵-Xaa⁶-Xaa⁷-Xaa⁸-ψ-Xaa⁹-NH₂ (A) wherein:
Xaa¹ is an N-terminal amino acid residue selected from D-Phe, 4-chlorophenylalanine (Cpa), D-Cpa, 3-(1-naphthyl)alanine (Nal), D-Tpi, D-Nal, 3-(2-naphthyl)alanine (2-Nal), or D-2-Nal;
Xaa² is Asn, Gln, homoserine (Hse), citrulline (Cit) or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(5-OH), Tpi, 7-Aza, or αMe-Trp;
Xaa⁴ is Ala or Ser;
Xaa⁵ is Val, 2,3-dehydro-Val, Cpg (cyclopentylglycine), L-cyclopropylglycine, L-cyclobutylglycine, or tert-leucine (Tle);
Xaa⁶ is Gly, NMe-Gly, or D-Ala;
Xaa⁷ is His or NMe-His;
Xaa⁸ is Leu, D-Pro, or Phe;
Xaa⁹-NH₂ is a C-terminally amidated amino acid residue selected from Pro, Phe, oxazolidine-4-carboxylic acid (4-oxa-L-Pro), Me₂Thz (5,5-dimethyl-1,3-thiazolidine-4-carboxylic acid), or thiazoline-4-carboxylic acid (Thz);
ψ represents a peptide bond or reduced peptide bond joining Xaa⁸ to Xaa⁹;
excluding compounds in which Xaa², Xaa³, Xaa⁵, and Xaa⁷ are Gln, Trp, Val, and His, respectively, in which Ly is a reduced peptide bond;
R^{L} is —C(O)—, —NH—C(O)—, or —NH—C(S)—;
the linker is a linear or branched chain of n1 units of -L¹R¹— and/or -(L¹)₂R¹—, wherein:
n1 is 1-20;
each R¹ is, independently, a linear, branched, and/or cyclic Cₙ₂ alkylenyl, alkenylenyl and/or alkynylenyl, wherein each n2 is independently 1-20, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid;
L¹ bonds to carbon, wherein each L¹ is independently —S—, —N(R²)C(O)—, —C(O)N(R²)—, —NH—C(O)—NH—, —NH—C(S)—NH—, 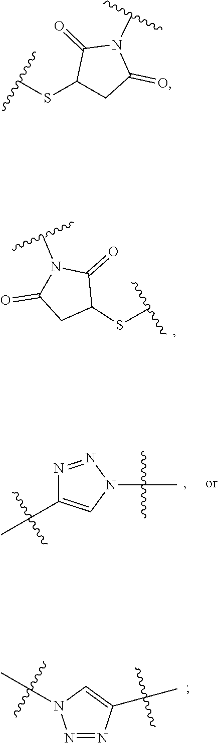 [SMILES: *SC1CC(=O)N(*)C1=O ; *SC1CC(=O)N(*)C1=O ; *c1cn(*)nn1 ; *c1cn(*)nn1]
and
R² is H, methyl or ethyl; and
an albumin binder (R^{alb}) is optionally bonded to an L¹ of the linker, wherein the albumin binder is:
—(CH₂)ₙ₃—CH₃ wherein n3 is 8-20;
—(CH₂)ₙ₄—C(O)OH wherein n4 is 8-20; 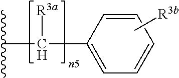
wherein n5 is 1-4 and R^{3a} is H or methyl, and R^{3b} is I, Br, F, Cl, H, OH, OCH₃, NH₂, NO₂ or C₁-C₆ alkyl; or 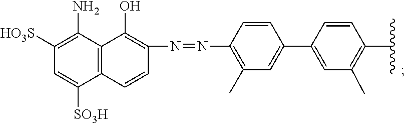 [SMILES: *c1ccc(-c2ccc(N=Nc3ccc4c(S(=O)(=O)O)cc(S(=O)(=O)O)c(N)c4c3O)c(C)c2)cc1C]
n6 is 1-5; and
each R^{rad} is a radiolabeling group bonded to or incorporating an L¹ of the linker, wherein each radiolabeling group is independently: a radiometal chelator; an aryl or heteroaryl substituted with a radiohalogen; a prosthetic group containing a trifluoroborate; a prosthetic group containing a silicon-fluorine-acceptor moiety; or a prosthetic group containing a fluorophosphate, fluorosulfate, sulfonyl fluoride, or a combination thereof.

2. The peptidic compound of claim 1, wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), or D-2-Nal;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(5-OH), or αMe-Trp;
and Xaa⁵ is Val, Cpg (cyclopentylglycine), or tert-leucine (Tle).

3. The peptidic compound of claim 1 or 2, wherein Xaa¹ is an N-terminal amino acid residue selected from D-Phe, or D-2-Nal;

4. The peptidic compound of any one of claims 1-3 wherein Xaa² is Gln, or His.

5. The peptidic compound of any one of claims 1-4, wherein Xaa⁵ is Val, or tert-leucine (Tle).

6. The peptidic compound of any one of claims 1-5, wherein Xaa⁶ is Gly, or NMe-Gly.

7. The peptidic compound of any one of claims 1-6, wherein Xaa⁹-NH₂ is a C-terminally amidated amino acid residue selected from Pro or thiazoline-4-carboxylic acid (Thz).

8. The peptidic compound of any one of claims 1-7, wherein:
Xaa¹ is an N-terminal amino acid residue selected from D-Phe, or D-2-Nal;
Xaa² is Gln, or His;
Xaa⁴ is Ala;
Xaa⁵ is Val, or tert-leucine (Tle);
Xaa⁶ is Gly, or NMe-Gly;
Xaa⁸ is Leu; and
Xaa⁹-NH₂ is a C-terminally amidated amino acid residue selected from Pro or thiazoline-4-carboxylic acid (Thz).

9. The peptidic compound of any one of claim 1 or 3-8, wherein Xaa³ is β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(5-OH), Tpi, 7-Aza, or αMe-Trp.

10. The peptidic compound of any one of claims 1-9, wherein ψ is a peptide bond.

11. The peptidic compound of any one of claims 1 to 10, wherein Xaa⁹ is Thz.

12. The peptidic compound of any one of claims 1 to 11, wherein Xaa² is His.

13. The peptidic compound of any one of claims 1 to 8 or 10-12, wherein Xaa³ is Trp.

14. The peptidic compound of any one of claims 1 to 8 or 10-13, wherein Xaa⁵ is Tle.

15. The peptidic compound of any one of claims 1 to 8 or 10-16, wherein Xaa⁷ is NMe-His.

16. The peptidic compound of any one of claims 1 to 8 or 10-15, wherein:
ψ is a peptide bond;
Xaa⁹ is Thz;
Xaa² is His;
Xaa³ is Trp;
Xaa⁵ is Tle; and
Xaa⁷ is NMe-His.

17. The peptidic compound of any one of claims 1-12 or 14-15, wherein Xaa³ is αMe-Trp.

18. The peptidic compound of any one of claims 1 to 17, wherein Xaa⁶ is Gly.

19. The peptidic compound of any one of claims 1 to 18, wherein Xaa⁸ is Leu.

20. The peptidic compounds of any one of claims 1-19, wherein at least one of Xaa¹, Xaa², Xaa³, Xaa⁴, Xaa⁵, Xaa⁶, Xaa⁷, Xaa⁸, or Xaa⁹ is methylated.

21. The peptidic compounds of any one of claims 1-20, wherein the peptidic compounds do not include an albumin binder R^{alb}.

22. A peptidic compound, wherein the compound has the structure of Formula B or is a salt or solvate of Formula B,
R^{rad}ₙ₆-[linker]-R^{L}-Xaa¹-Xaa²-Xaa³-Xaa⁴-Xaa⁵-Xaa⁶-Xaa⁷-Xaa⁸-ψ-Xaa⁹-NH₂ (B) wherein:
Xaa¹ is an N-terminal amino acid residue selected from D-Phe, 4-chlorophenylalanine (Cpa), D-Cpa, 3-(1-naphthyl)alanine (Nal), D-Tpi, D-Nal, 3-(2-naphthyl)alanine (2-Nal), or D-2-Nal;
Xaa² is Asn, Gln, homoserine (Hse), citrulline (Cit) or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(5-OH), Tpi, 7-Aza, or αMe-Trp;
Xaa⁴ is Ala or Ser;
Xaa⁵ is Val, 2,3-dehydro-Val, Cpg (cyclopentylglycine), L-cyclopropylglycine, cyclobutylglycine, or tert-leucine (Tle);
Xaa⁶ is Gly, NMe-Gly, or D-Ala;
Xaa⁷ is His or NMe-His;
Xaa⁸ is Leu, D-Pro, or Phe;
Xaa⁹-NH₂ is a C-terminally amidated amino acid residue selected from Pro, oxazolidine-4-carboxylic acid (4-oxa-L-Pro), Me₂Thz (5,5-dimethyl-1,3-thiazolidine-4-carboxylic acid), or thiazoline-4-carboxylic acid (Thz);
ψ represents a peptide bond or reduced peptide bond joining Xaa⁸ to Xaa⁹;
R^{L} is —C(O)—, —NH—C(O)—, or —NH—C(S)—;
the linker is a linear or branched chain of n1 units of -L¹R¹— and/or -(L¹)₂R¹—, wherein:
n1 is 1-20;
each R¹ is, independently, a linear, branched, and/or cyclic Cₙ₂ alkylenyl, alkenylenyl and/or alkynylenyl, wherein each n2 is independently 1-20, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid;
L¹ bonds to carbon, wherein each L¹ is independently —S—, —N(R²)C(O)—, —C(O)N(R²)—, —NH—C(O)—NH—, —NH—C(S)—NH—, 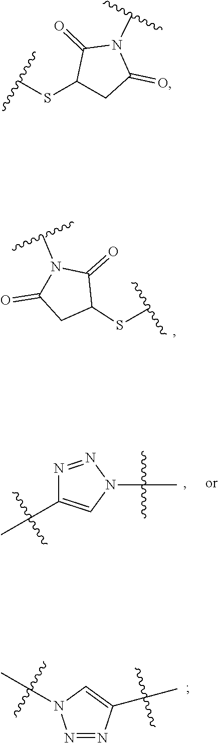 [SMILES: *SC1CC(=O)N(*)C1=O ; *SC1CC(=O)N(*)C1=O ; *c1cn(*)nn1 ; *c1cn(*)nn1]
and
R² is H, methyl or ethyl; and
an albumin binder (R^{alb}) is optionally bonded to an L¹ of the linker, wherein the albumin binder is:
—(CH₂)ₙ₃—CH₃ wherein n3 is 8-20;
—(CH₂)ₙ₄—C(O)OH wherein n4 is 8-20; 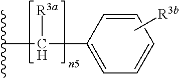
wherein n5 is 1-4 and R^{3a} is H or methyl, and R^{3b} is I, Br, F, Cl, H, OH, OCH₃, NH₂, NO₂ or C₁-C₆ alkyl; or 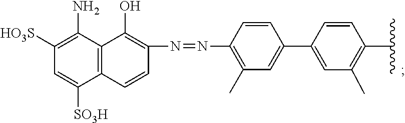 [SMILES: *c1ccc(-c2ccc(N=Nc3ccc4c(S(=O)(=O)O)cc(S(=O)(=O)O)c(N)c4c3O)c(C)c2)cc1C]
n6 is 1-5; and
each R^{rad} is a radiolabeling group bonded to or incorporating an L¹ of the linker, wherein each radiolabeling group is independently: a radiometal chelator; an aryl or heteroaryl substituted with a radiohalogen; a prosthetic group containing a trifluoroborate; a prosthetic group containing a silicon-fluorine-acceptor moiety; or a prosthetic group containing a fluorophosphate, fluorosulfate, sulfonyl fluoride, or a combination thereof.

23. The peptidic compound of claim 22, wherein at least one of Xaa¹, Xaa², Xaa³, Xaa⁴, Xaa⁵, Xaa⁶, Xaa⁷, Xaa⁸, or Xaa⁹ is methylated.

24. The peptidic compound of any one of claims 22-23, wherein y is reduced peptide bond joining Xaa⁸ to Xaa⁹.

25. The peptidic compound of any one of claims 22-24, wherein:
Xaa¹ is D-Phe; and/or
Xaa⁶ is Gly; and/or
Xaa⁸ is Leu; and/or
Xaa⁹ is Pro, Thz or 4-oxa-L-Pro.

26. The peptidic compound of any one of claims 22-25, wherein Xaa⁶ is Gly or N-methyl-Gly.

27. The peptidic compound of any one of claims 22-26, wherein Xaa⁹ is Thz.

28. The peptidic compound of any one of claims 22-26, wherein Xaa⁹ is Pro.

29. The peptidic compound of claim 22, wherein Xaa¹ is D-Phe, Xaa² is Gln, Xaa³ is Trp, Xaa⁴ is Ala, Xaa⁵ is Val, Xaa⁶ is Gly or N-methyl-Gly, Xaa⁷ is His, Xaa⁸ is Leu, Xaa⁹ is Thz, and L is a reduced peptide bond joining Xaa⁸ to Xaa⁹.

30. The peptidic compound of claims 26, 27, 28 or 29, wherein Xaa⁶ is N-methyl-Gly.

31. The peptidic compounds of any one of claims 22-30, wherein the peptidic compounds do not include an albumin binder R^{alb}.

32. The peptidic compound of any one of claims 1-20 or 22-30, wherein R^{rad}ₙ₆-[linker]-R^{L}— is DOTA-Pip-.

33. The peptidic compound of any one of claims 1 to 32, wherein at least one R^{rad} is a radiometal chelator, optionally selected from the group consisting of: DOTA and derivatives; DOTAGA; NOTA; NODAGA; NODASA; CB-DO2A; 3β-C-DEPA; TCMC; DO3A; DTPA and DTPA analogues optionally selected from CHX-A″-DTPA and 1B4M-DTPA; TETA; NOPO; Me-3,2-HOPO; CB-TE1A1P; CB-TE2P; MM-TE2A; DM-TE2A; sarcophagine and sarcophagine derivatives optionally selected from SarAr, SarAr-NCS, diamSar, AmBaSar, and BaBaSar; TRAP; AAZTA; DATA and DATA derivatives; H2-macropa or a derivative thereof; H₂dedpa, H₄octapa, H₄py4pa, H₄Pypa, H₂azapa, H₅decapa, and other picolinic acid derivatives; CP256; PCTA; C-NETA; C-NE3TA; HBED; SHBED; BCPA; CP256; YM103; desferrioxamine (DFO) and DFO derivatives; H₆phospa; a trithiol chelate; mercaptoacetyl; hydrazinonicotinamide; dimercaptosuccinic acid; 1,2-ethylenediylbis-L-cysteine diethyl ester; methylenediphosphonate; hexamethylpropyleneamineoxime; hexakis(methoxy isobutyl isonitrile), H4py4pa-phenyl-NCS, and Crown.

34. The peptidic compound of claim 33, wherein the radiometal chelator is bound by a radiometal, a radionuclide-bound metal, or a radionuclide-bound metal-containing prosthetic group, optionally selected from the group consisting of: ⁶⁸Ga, ⁶¹Cu, ⁶⁴Cu, ⁶⁷Cu, ⁶⁷Ga, ¹¹¹In, ⁴⁴Sc, ⁸⁶Y, ⁸⁹Zr, ⁹⁰Nb, ¹⁷⁷Lu, ^{117m}Sn, ¹⁶⁵Er, ⁹⁰Y, ²²⁷Th, ²²⁵Ac, ²¹³Bi, ²¹²Bi, ⁷²As, ⁷⁷As, ²¹¹At, ²⁰³Pb, ²¹²Pb, ⁴⁷Sc, ¹⁶⁶Ho, ¹⁸⁸Re, ¹⁸⁶Re, ¹⁴⁹Pm, ¹⁵⁹Gd, ¹⁰⁵Rh, ¹⁰⁹Pd, ¹⁹⁸Au, ¹⁹⁹Au, ¹⁷⁵Yb, ¹⁴²Pr, ^{114m}In, ^{94m}Tc, ^{99m}Tc, ¹⁴⁹Tb, ¹⁵²Tb, ¹⁵⁵Tb, ¹⁶¹Tb, and [¹⁸F]AlF.

35. The peptidic compound of any one of claims 1 to 34, wherein at least one R^{rad} is a trifluoroborate containing prosthetic group BF₃—R⁵—R⁴—, wherein R⁴ is —(CH₂)₁₋₅— and optionally methylene, and wherein BF₃—R⁵— forms: 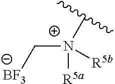
wherein R^{5a} and R^{5b} are each independently a C₁-C₅ linear or branched alkyl group, 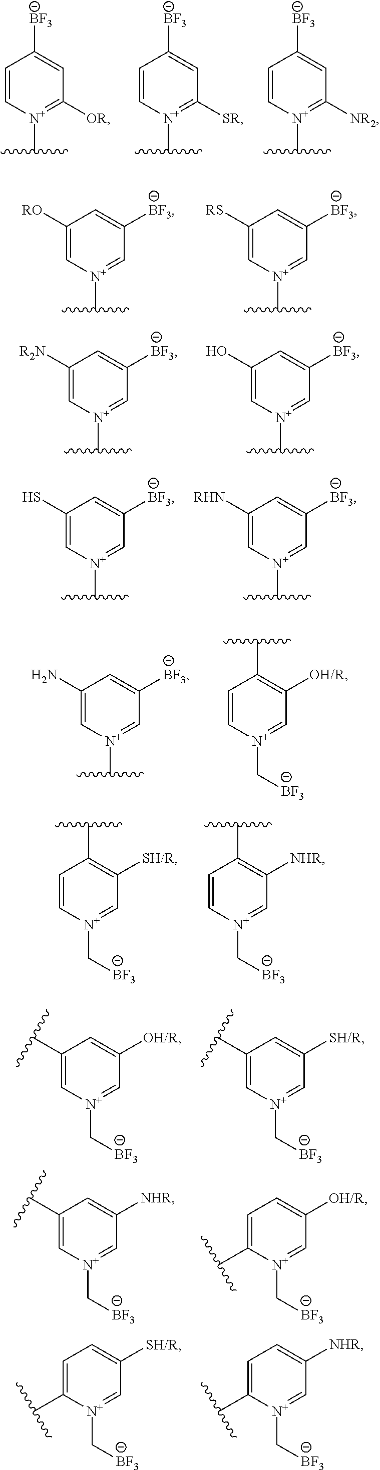 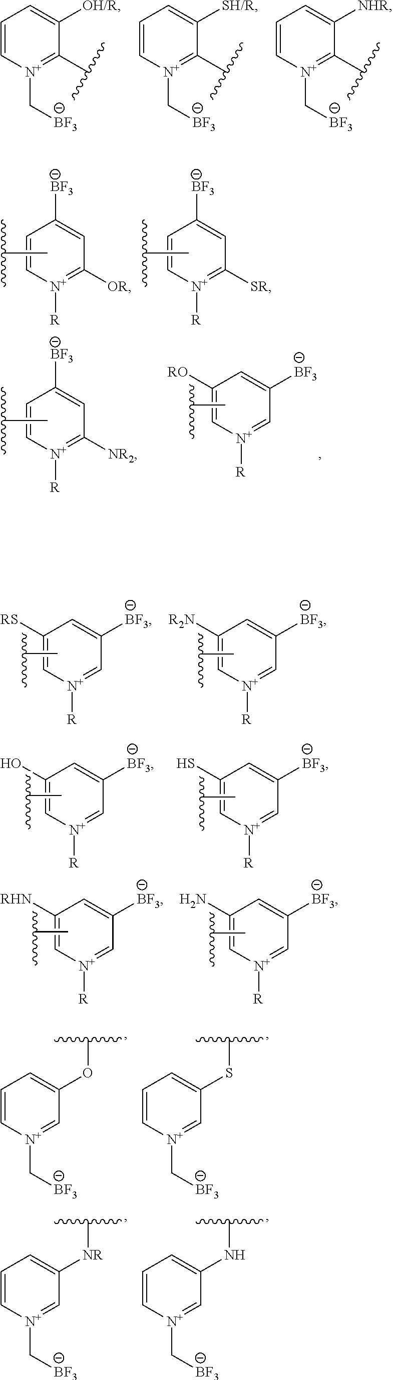 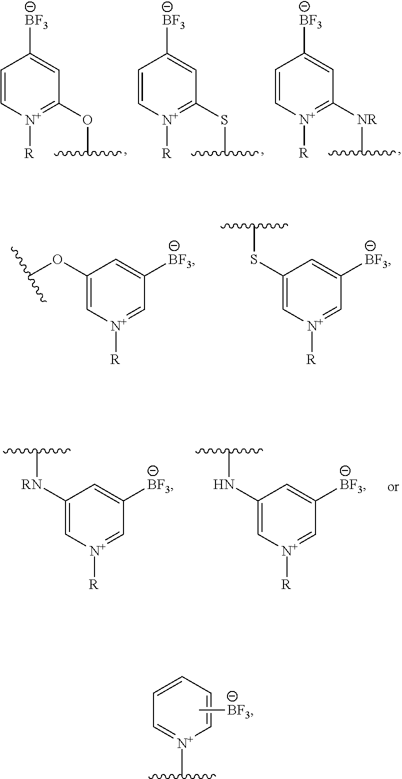
in which the R in each pyridine substituted —OR, —SR, —NR—, —NHR or —NR₂ is independently a branched or linear C₁-C₅ alkyl, optionally wherein the fluorines in BF₃—R⁵—R⁴— comprise ¹⁸F.

36. The peptidic compound of any one of claims 1 to 35, wherein the linker and R^{L} together form a linear or branched peptide linker (Xaa¹⁰)₁₋₂₀, wherein each Xaa¹⁰ is independently a proteinogenic or non-proteinogenic amino acid residue, wherein each peptide backbone amino group is independently optionally methylated, and wherein each non-proteinogenic amino acid residue is independently selected from Table 1.

37. The peptidic compound of any one of claims 1 to 36, wherein n6 is 1, and wherein the linker and R^{L} together form a p-aminomethylaniline-diglycolic acid (pABzA-DIG) linker, a 4-amino-(1-carboxymethyl)piperidine (Pip) linker, a 9-amino-4,7-dioxanonanoic acid (dPEG2) linker, or a 4-(2-aminoethyl)-1-carboxymethyl-piperazine Ac) linker, optionally wherein the linker and R^{L} together form:

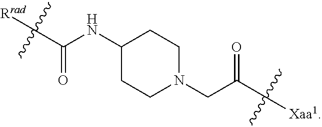

38. The peptidic compound of any one of claims 1-33 or 35-37, wherein the radiometal, the radionuclide-bound metal, or the radionuclide-bound metal-containing prosthetic group is: ⁶⁸Ga, ⁶¹Cu, ⁶⁴Cu, ⁶⁷Ga, ^{99m}Tc, ^{110m}In, ¹¹¹In, ⁴⁴Sc, ⁸⁶Y, ⁸⁹Zr, ⁹⁰Nb, ¹⁵²Tb, ¹⁵⁵Tb, [¹⁸F]AlF, ¹³¹I, ¹²³I, ¹²⁴I, ²⁰³Pb and ⁷²As.

39. The peptidic compound of any one of claims 1-33 or 35-37, wherein the radiometal, the radionuclide-bound metal, or the radionuclide-bound metal-containing prosthetic group is: ¹⁶⁵Er, ²¹²Bi, ²¹¹At, ¹⁶⁶Ho, ¹⁴⁹Pm, ¹⁵⁹Gd, ¹⁰⁵Rh, ¹⁰⁹Pd, ¹⁹⁸Au, ¹⁹⁹Au, ¹⁷⁵Yb, ¹⁴²Pr, ¹⁷⁷Lu, ¹¹¹In, ²¹³Bi, ⁴⁷Sc, ⁹⁰Y, ²²⁵Ac, ^{117m}Sn, ¹⁵³Sm, ¹⁴⁹Tb, ¹⁶¹Tb, ²²⁴Ra, ²²³Ra, ²¹²Pb, ²²⁷Th, ²²³Ra, ⁷⁷As, ¹⁸⁶Re, ¹⁸⁸Re, ⁶⁷Cu, or ⁶⁴Cu.

40. A method of imaging Gastrin-releasing peptide receptor (GRPR) in a subject, the method comprising: administering to the subject a peptidic compound of any one of claims 1-38; and imaging tissue of the subject.

41. A method of treating cancer in a subject comprising, administering to the subject in need thereof a peptidic compound of any one of claims 1-37 or 39 and one or more pharmaceutically acceptable excipients.
